(19) United States
(12) Patent Application Publication
(10) Pub. No.: US 2010/0029620 A1
(43) Pub. Date: Feb. 4, 2010

(54) SUBSTITUTED TRIAZOLINE, TETRAZOLONE AND IMIDAZOLONE DERIVATIVES FOR USE AS A MEDICINE

(75) Inventors: José Ignacio Andrés-Gil, Madrid (ES); Manuel Jesús Alcázar-Vaca, Toledo (ES); Joaquin Pastor-Fernández, Toledo (ES); Xavier Jean,Michel Langlois, Beerse (BE); Julen Oyarzabal-Santamarina, Toledo (ES); Juan Antonio Vega-Ramiro, Toledo (ES)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JANSSEN PHARMACEUTICA N.V.

(21) Appl. No.: 11/722,380

(22) PCT Filed: Dec. 20, 2005
(86) PCT No.: PCT/EP05/56951

§ 371 Date: Jun. 21, 2007

(30) Foreign Application Priority Data
Dec. 21, 2004 (EP) 04106817.2
Jun. 3, 2005 (EP) 05104873.4

(51) Int. Cl. A61K 31/496; C07D 401/14; C07D 405/14; C07D 413/14; C07D 403/10; C07D 401/04; A61K 31/454; A61K 31/5377; A61K 31/551; A61P 25/22; A61P 25/24; A61P 25/00; A61P 25/28; A61P 25/16

(52) U.S. Cl. 514/218; 546/201; 544/364; 544/130; 544/366; 546/210; 540/575; 514/323; 514/326; 514/253.09; 514/236.2; 514/254.05

(57) ABSTRACT

The present invention concerns substituted triazolone, tetrazolone and imidazolone derivatives according to the general Formula (I)

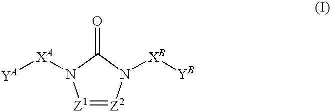

a pharmaceutically acceptable acid or base addition salt thereof, a stereochemically isomeric form thereof, an N-oxide form thereof or a quaternary ammonium salt thereof, wherein the variables are defined in Claim 1, having selective α_{2C}-adrenoceptor antagonist activity. It further relates to their preparation, compositions comprising them and their use as a medicine. The compounds according to the invention are useful for the prevention and/or treatment of central nervous system disorders, mood disorders, anxiety disorders, stress-related disorders associated with depression and/or anxiety, cognitive disorders, personality disorders, schizoaffective disorders, Parkinson's disease, dementia of the Alzheimer's type, chronic pain conditions, neurodegenerative diseases, addiction disorders, mood disorders and sexual dysfunction.

### FIELD OF THE INVENTION

The present invention concerns substituted triazolone, tetrazolone and imidazolone derivatives having selective α_{2C}-adrenoceptor antagonist activity. It further relates to their preparation, compositions comprising them and their use as a medicine.

### BACKGROUND OF THE INVENTION

Adrenergic receptors form the interface between the endogenous catecholamines epinephrine and norepinephrine and a wide array of target cells in the body to mediate the biological effects of the sympathetic nervous system. They are divided into three major subcategories, α₁, α₂ and β. To date, nine distinct adrenergic receptor subtypes have been cloned from several species: α_{1A}, α_{1B}, α_{1D}, α_{2A}, α_{2B}, α_{2C}, β₁, β₂ and β₃ (Hieble, J. P.; et al. J. Med. Chem. 1995, 38, 3415-3444). Available α₂ ligands have only marginal subtype selectivity. A complicating factor is that α₂-adrenoceptor ligands, which are imidazoles or imidazolines, also bind with moderate-to-high affinity to non-adrenoceptor imidazoline binding sites.

The three α₂-adrenoceptor subtypes share many common properties. They are G-protein-coupled receptors with seven transmembrane domains of the aminebinding subfamily. All three subtypes are coupled to the Gi/o signalling system, inhibiting the activity of adenylate cyclase, the opening of voltage-gated Ca²⁺ channels and the opening of K⁺ channels. The three receptors are encoded by distinct genes (Bylund, D. B.; et al. Pharmacol. Rev. 1994, 46, 121-136 and Hieble, J. P. et al. Pharmacol. Commun. 1995, 6, 91-97), localized to different chromosomes; in humans the gene for α_{2A} is found on chromosome 10, the α_{2B}-gene on chromosome 2 and the α_{2C}-gene on chromosome 4. The subtypes are well conserved across mammalian species. In rats and mice, however, there is a single amino acid substitution which decreases the affinity of the rodent α_{2A}-adrenoceptor for the classical α₂-antagonists, yohimbine and rauwolscine. The general consensus is that this so-called α_{2D}-adrenoceptor subtype represents the rodent homologue of the human α_{2A}-subtype.

The α₂-adrenoceptor subtypes are differentially distributed in cells and tissues, clearly endowing the receptors with different physiological functions and pharmacological activity profiles. Different regulatory regions in the receptor genes and different protein structures also confer different regulatory properties on the three receptors, both with regard to receptor synthesis and post-translational events.

α₂-Adrenergic receptors were initially characterized as presynaptic receptors that serve as parts of a negative feedback loop to regulate the release of norepinephrine. Soon it was shown that α₂-adrenoceptors are not restricted to presynaptic locations but also have postsynaptic functions. The α_{2A}-adrenoceptor is the major inhibitory presynaptic receptor (autoreceptor) regulating release of norepinephrine from sympathetic neurons as part of a feedback loop. The α_{2C}-adrenoceptor turned out to function as an additional presynaptic regulator in all central and peripheral nervous tissues investigated. However, the relative contributions of α_{2A} and α_{2C}-receptors differed between central and peripheral nerves, with the α_{2C}-subtype being more prominent in sympathetic nerve endings than in central adrenergic neurons (Philipp, M. et al. Am. J. Physiol. ReguL Integr. Comput. Physiol. 2002, 283, R287-R295 and Kable, J. W. et al. J. Pharmacol. Exp. Ther. 2000, 293, 1-7). The α_{2C}-adrenoceptor is particularly suited to control neurotransmitter release at low action potential frequencies. In contrast, the α_{2A}-adrenoceptor seems to operate primarily at high stimulation frequencies in sympathetic nerves and may thus be responsible for controlling norepinephrine release during maximal sympathetic activation (Bucheler, M. M. et al. Neuroscience 2002, 109, 819-826). α_{2B}-Adrenoceptors are located on postsynaptic cells to mediate the effects of catecholamines released from sympathetic nerves, e.g., vasoconstriction. α₂-Adrenergic receptors not only inhibit release of their own neurotransmitters but can also regulate the exocytosis of a number of other neurotransmitters in the central and peripheral nervous system. In the brain, α_{2A}- and α_{2C}-adrenoceptors can inhibit dopamine release in basal ganglia as well as serotonin secretion in mouse hippocampal or brain cortex slices. In contrast, the inhibitory effect of α₂-adrenoceptor agonists on gastrointestinal motility was mediated solely by the α_{2A}-subtype. Part of the functional differences between α_{2A}- and α_{2C}-receptors may be explained by their distinct subcellular localization patterns. When expressed in rat fibroblasts, α_{2A}- and α_{2B}-adrenoceptors are targeted to the plasma membrane. On stimulation with agonist, only α_{2B}-adrenoceptors are reversibly internalized into endosomes. α_{2C}-Adrenoceptors are primarily localized in an intracellular membrane compartment, from where they can be translocated to the cell surface after exposure to cold temperature (see a.o. Docherty J. R. et. al. Eur. J. Pharmacol. 1998, 361, 1-15).

The establishment of genetically engineered mice lacking or overexpressing α₂-adrenoceptor subtypes has yielded important information for understanding the subtype specific functions (MacDonald, E. et al. Trends Pharmacol. Sci. 1997, 18, 211-219).

The examination of the phenotype of these strains of mice demonstrated that the α_{2A} subtype is responsible for inhibition of neurotransmitter release from central and peripheral sympathetic nerves and for most of the centrally mediated effects of α₂-agonists. The α_{2B} subtype is primarily responsible for the initial peripheral hypertensive responses evoked by the α₂-agonists and takes part in the hypertension induced by salt (Link et al. Science 1996, 273, 803-805 and Makaritsis, K. P. et al. Hypertension 1999, 33, 14-17).

Clarification of the physiological role of the α_{2C} subtype proved more difficult. Despite a rather wide distribution in the CNS, its role did not appear critical in the mediation of the cardiovascular effects of nonselective α₂-agonists. Its participation has been suggested in the hypothermia induced by dexmedetomidine and in the hyperlocomotion induced by D-amphetamine (Rohrer, D. K. et al. Annu. Rev. Pharmacol Toxicol. 1998, 38, 351-373). Another potentially important response mediated by the α_{2C}-adrenoceptor is constriction of cutaneous arteries, leading to a reduction in cutaneous blood flow (Chotani, M. A. et al. Am. J. Physiol. Heart Circ. Physiol. 2004, 286, 59-67). Recent studies carried out on double knockout mice have suggested that α_{2C}-adrenoceptor is also expressed at the presynaptic level where, together with α_{2A}, it actively participates in the control of neurotransmitter release. While α_{2A}-adrenoceptor is particularly efficient at high stimulation frequencies, α_{2C}-adrenoceptor acts rather at low stimulation frequencies. Moreover, it has been suggested that α_{2C} subtype participates in the modulation of motor behavior and the memory processes (Bjorklund, M. et al. Neuroscience 1999, 88, 1187-1198 and Tanila, H. et al. Eur. J. Neurosci. 1999, 11, 599-603). Other central effects triggered by this subtype include also the startle reflex and aggression response to stress and locomotion (Sallinen, J. et al. J. Neurosci. 1998, 18, 3035-3042 and Sallinen. J. et al. Neuroscience 1998, 86, 959-965). Last, it was recently pointed out that the α_{2C}-adrenoceptor might contribute to α₂-agonist-mediated spinal analgesia and adrenergic-opioid synergy (Fairbanks, C. A. et al. J. Pharm. Exp. Ther. 2002, 300, 282-290).

Because of their widespread distribution in the central nervous system, α₂-receptors affect a number of behavioral functions. The effect of altered α_{2C}-adrenergic receptor expression has been evaluated in several different behavioral paradigms (Kable J. W. et al., Journal of Pharmacology and Experimental Therapeutics, 2000, 293 (1):

1-7), proving that α_{2C}-adrenergic antagonists may have therapeutic value in the treatment of stress-related psychiatric disorders. In each of the behavioral paradigms, it is unclear whether the α_{2C}-subtype plays some direct role in mediating behavior or whether altered α_{2C}-receptor expression produces effects because of altered metabolism or downstream modulation of other neurotransmitter systems. Interestingly, α_{2C}-receptor-deficient mice had enhanced startle responses, diminished prepulse inhibition, and shortened attack latency in the isolation aggression test. Thus drugs acting via the α_{2C}-adrenoceptor may have therapeutic value in disorders associated with enhanced startle responses and sensorimotor gating deficits, such as schizophrenia, attention deficit disorder, posttraumatic stress disorder, and drug withdrawal. In addition to the α_{2C}-subtype, the α_{2A}-adrenoceptor has an important.

With more and more studies of the α₂-adrenoceptor physiology in gene-targeted mice being published, the situation becomes more complicated than initially anticipated. Indeed, only a few biological functions of α₂-receptors were found to be mediated by one single α₂-adrenergic receptor subtype. For other α₂-receptor-mediated functions, two different strategies seem to have emerged to regulate adrenergic signal transduction: some biological functions are controlled by two counteracting α₂-receptor subtypes, and some require two receptor subtypes with similar but complementary effects. Because the α_{2A}-subtype mediates most of the classical effects of α₂-adrenergic agonists, it is doubtful that an α_{2A}-selective agonist would have a substantially better clinical profile than the currently available agents. Drugs acting at α_{2B}- or α_{2C}-adrenergic receptors are likely to have fewer of the classical α₂-adrenergic side effects than α_{2A}-specific agents. It would appear likely that α_{2C}-selective agents may be useful in at least some nervous system disorders, in particular central nervous system disorders.

### BACKGROUND PRIOR ART

Analysis of the pipeline databases to date indicate that there are several adrenergic α₂-antagonists in the market, by companies including Akzo Nobel (Organon), Novartis, Pfizer, and Schering AG. None of those compounds are selective for any of the three α₂-adrenoceptors. These compounds are indicated mainly for depression, hypertensive disorders and dyskinesias associated with Parkinson's disease. Companies with α₂-adrenoceptor antagonists in clinical development include Britannia Pharmaceuticals, IVAX, Juvantia Pharmaceuticals, MAP Pharmaceuticals, Novartis, Novo Nordisk, Organon, Pierre Fabre, and Sanofi-Aventis.

Regarding the development of selective α_{2C}-adrenoceptor antagonists to date, OPC-28326 is the only compound in clinical development (in Phase 2 by Otsuka Pharmaceuticals for hypertensive disorders and peripheral vascular disease). The rest of the α_{2C} antagonists are in preclinical development by Oy Juvantia Pharma Ltd (JP 1514 and JP 1302, published in WO 01/64645 and WO 04/067513) and by Novartis AG (NVP-ABE651 and NVP-ABE697, published in WO 01/55132 and J. Label Compd. Radiopharm 2002, 45, 1180), indicated mainly for depression and schizophrenia. In addition, several compounds are listed at the very early stages of development (biological testing) by Juvantia and Kyowa Hakko, for depression and Parkinson's disease.

### DESCRIPTION OF THE INVENTION

It is the object of the present invention to provide compounds with a binding affinity towards α₂-adrenoceptor receptors, in particular towards α_{2C}-adrenoceptor receptors, in particular as an antagonist.

This goal was achieved by a novel substituted triazolone, imidazolone and tetrazolone derivative according to the general Formula (I)

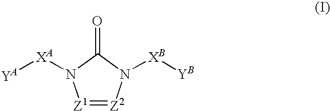

a pharmaceutically acceptable acid or base addition salt thereof, a stereochemically isomeric form thereof, an N-oxide form thereof or a quaternary ammonium salt thereof, wherein
Z¹ and Z² each independently from each other are CH or N;
X^{A} and X^{B} each independently from each other are a covalent bond or a C₁₋₄alkyl-radical, wherein one bivalent —CH₂-unit may be replaced by a bivalent phenyl-unit and/or wherein one or more hydrogen atoms in each moiety X^{A} and X^{B} may be replaced by a radical selected from the group of halo, cyano, hydroxy, amino, oxo and formyl;
Y^{A} and Y^{B} each independently from each other are a radical selected from the group of t-butyl, NR¹R² and Pir;
R¹ and R² each independently from each other are a radical selected from the group of hydrogen; alkyl; aryl; aryloxy; Het; —NR^{a}R^{b}, wherein R^{a} and R^{b} each independently are hydrogen, alkyl, aryl or arylalkyl; and alkyl substituted with one or more radicals selected from the group of aryl, aryloxy, Het and —NR^{a}R^{b}, wherein R^{a} and R^{b} each independently are selected from the group of hydrogen, alkyl, aryl and arylalkyl;
Pir is a radical selected from the group of pyrrolyl; pyrazolyl; imidazolyl pyridinyl; pyrimidinyl; pyrazinyl; pyridazinyl; pyrrolidinyl imidazolidinyl; pyrrazolidinyl; piperidinyl; diazepyl; morpholinyl thiomorpholinyl; piperazinyl; imidazolidinyl; 2H-pyrrolyl; pyrrolinyl imidazolinyl; pyrrazolinyl; 1,2,3,4-tetrahydro-isoquinolinyl; 7,9-diaza-bicyclo[4.2.2]dec-3-enyl and isoindolyl; wherein each Pir-radical may optionally be substituted by one or more radicals selected from the group of hydroxy; oxo; alkyl; alkylcarbonyl; alkylsulphonyl alkyloxycarbonyl; aryloxyalkyl; mono-arylaminoalkyl; aryl; arylalkyl arylalkenyl; pyrrolidinyl; furylalkyl optionally substituted with 1 or 2 alkyl radicals; benzofurylalkyl; 2,3-dihydro-benzo[1,4]dioxylalkyl; quinolinylalkyl; benzothienylalkyl and indolylalkyl, optionally substituted with halo;
Het is a monocyclic heterocyclic radical selected from the group of pyrrolyl, pyrazolyl, imidazolyl, pyridinyl, pyrimidinyl, pyrazinyl, pyridazinyl, pyrrolidinyl, imidazolidinyl, pyrrazolidinyl, piperidinyl, diazepyl, morpholinyl, thiomorpholinyl, piperazinyl, imidazolidinyl, 2H-pyrrolyl, pyrrolinyl, imidazolinyl, pyrrazolinyl, furanyl, thienyl, oxazolyl, isoxazolyl, thiazolyl, thiadiazolyl, isothiazolyl, dioxolyl, dithianyl, tetrahydrofuryl, triazolyl and triazinyl; or a bicyclic heterocyclic radical selected from the group of quinolinyl, isoquinolinyl, 1,2,3,4-tetrahydro-isoquinolinyl, quinoxalinyl, indolyl, isoindolyl, benzimidazolyl, benzoxazolyl, benzisoxazolyl, benzothiazolyl, benzisothiazolyl, benzofuranyl, benzothienyl, benzopiperidinyl, chromenyl and imidazo[1,2-a]pyridinyl; wherein each Het-radical is optionally substituted with alkyl;
   or two adjacent moieties X and Y may be fused together to form the bivalent radical 1,2,3,4-tetrahydro-isoquinolinyl, optionally substituted with hydrogen, alkyl, aryl, arylalkyl, alkylcarbonyl, alkylsulphonyl and pyrrolidinylalkyl;
aryl is naphthalenyl or phenyl, each optionally substituted with 1, 2 or 3 substituents, each independently from each other, selected from the group of halo, cyano, hydroxy, amino, alkylamino, alkyloxyalkylamino, oxo, carboxy, nitro, thio, formyl and alkyloxy;
alkyl is a straight or branched saturated hydrocarbon radical having from 1 to 6 carbon atoms; or is a cyclic saturated hydrocarbon (cycloalkyl) radical having from 3 to 7 carbon atoms; or is a cyclic saturated hydrocarbon radical having from 3 to 7 carbon atoms attached to a straight or branched saturated hydrocarbon radical having from 1 to 6 carbon atoms; each radical may optionally be substituted on one or more carbon atoms with one or more radicals selected from the group of halo, cyano, hydroxy, amino, oxo, carboxy, nitro, thio and formyl; and
alkenyl is an alkyl radical as defined above further having one or more double bonds.

The invention also relates to a pharmaceutical composition comprising a pharmaceutically acceptable carrier or diluent and, as active ingredient, a therapeutically effective amount of a compound according to the invention, in particular a compound according to Formula (I), a pharmaceutically acceptable acid or base addition salt thereof, a stereochemically isomeric form thereof, an N-oxide form thereof or a quaternary ammonium salt thereof.

The invention also relates to the use of a compound according to the invention for the preparation of a medicament for the prevention and/or treatment of a disorder or disease responsive to antagonism of the α₂-adrenergic receptor, in particular to antagonism of the α_{2C}-adrenergic receptor.

In particular, the invention relates to the use of a compound according to the invention for the preparation of a medicament for the prevention and/or treatment of central nervous system disorders, mood disorders, anxiety disorders, stress-related disorders associated with depression and/or anxiety, cognitive disorders, personality disorders, schizoaffective disorders, Parkinson's disease, dementia of the Alzheimer's type, chronic pain conditions, neurodegenerative diseases, addiction disorders, mood disorders and sexual dysfunction.

The compounds according to the invention may also be suitable as add-on treatment and/or prophylaxis in the above listed diseases in combination with antidepressants, anxiolytics and/or antipsychotics which are currently available or in development or which will become available in the future, to improve efficacy and/or onset of action. This is evaluated in rodent models in which antidepressants, anxiolytics and/or antipsychotics are shown to be active. For example, compounds are evaluated in combination with antidepressants, anxiolytics and/or antipsychotics for attenuation of stress-induced hyperthermia.

The invention therefore also relates to the use of the compounds according to the invention for use as an add-on treatment with one or more other compounds selected from the group of antidepressants, anxiolytics and antipsychotics, to a pharmaceutical composition comprising the compounds according to the invention and one or more other compounds selected from the group of antidepressants, anxiolytics and antipsychotics, as well as to a process for the preparation of such pharmaceutical compositions and to the use of such a composition for the manufacture of a medicament, in particular to improve efficacy and/or onset of action in the treatment of depression and/or anxiety.

### DETAILED DESCRIPTION OF THE INVENTION

In a preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein Z¹ is CH and Z² is N; or Z¹ is N and Z² is N; or Z¹ is CH and Z² is CH; or Z¹ is CH and Z² is CH.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein Z¹ is CH and Z² is N.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein each of X^{A} and X^{B}, independently from each other is selected from the group of a covalent bond, —CH₂—, —CH₂CH₂—, —CH₂CH₂CH₂—, —CH₂CH₂CH₂CH₂—, —CH(CH₃)CH(CH₃)—, —C(═O)CH₂—, —CH₂C(═O)—, —CH₂CH₂C(═O)CH₂—, —C₆H₄—, —CH₂C₆H₅—, —CH₂CH₂C₆H₅—, —C₆H₅CH₂—, —C₆H₅CH₂CH₂—, —CH₂C₆H₄CH₂— and —CH₂CH₂C₆H₄CH₂CH₂—.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein each of X^{A} and X^{B}, independently from each other are selected from the group of a covalent bond, —CH₂—, —CH₂CH₂—, —CH₂CH₂CH₂—, —CH(CH₃)CH(CH₃)—, —C(═O)CH₂—, —CH₂CH₂C(═O)CH₂—, —C₆H₄—, —CH₂C₆H₅—, —C₆H₅CH₂— and —CH₂C₆H₄CH₂—. Preferably, X^{A} and X^{B} are each independently from each other —CH₂CH₂— and —CH₂C₆H₅—. More preferably, X^{A} is —CH₂C₆H₅— and X^{B} is —CH₂CH₂—.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein Y^{A} is NR¹R² and Y^{B} is Pir; or Y^{A} is NR¹R² and Y^{B} is NR¹R²; or Y^{A} is Pir and Y^{B} is Pir; or Y^{A} is Pir and Y^{B} is NR¹R².

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein Y^{A} is Pir and Y^{B} is NR¹R².

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein Pir is selected from the group of pyrrolidinyl; piperidinyl; diazepyl; morpholinyl; piperazinyl; 1,2,3,4-tetrahydro-isoquinolinyl; 7,9-diaza-bicyclo[4.2.2]dec-3-enyl and isoindolyl; wherein each Pir-radical may optionally be substituted by one or more radicals selected from the group of hydroxy; oxo; alkyl; alkyloxycarbonyl; aryloxyalkyl, in particular phenyloxyethyl; mono-arylaminoalkyl, aryl; arylalkyl; arylalkenyl, in particular 1-(2-methyl-3-phenyl-allyl)pyrrolidinyl; furylalkyl, optionally substituted with 1 or 2 alkyl radicals; benzofurylalkyl; 2,3-dihydro-benzo[1,4]dioxylalkyl; quinolinylalkyl, benzothienyl-alkyl and indolylalkyl, optionally substituted with halo.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein Pir is morpholinyl.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein each of R¹ and R² independently from each other are selected from the group of hydrogen; alkyl; aryl and alkyl substituted with a radical selected from the group of aryl, aryloxy, Het and —NR^{a}R^{b}, wherein R^{a} and R^{b} each independently are selected from the group of hydrogen, alkyl and arylalkyl.

Preferably, the invention relates to a compound according to general Formula (I), a pharmaceutically acceptable acid or base addition salt thereof, a stereochemically isomeric form thereof, an N-oxide form thereof or a quaternary ammonium salt thereof, wherein Het is selected from the group of pyridinyl, pyrrolidinyl, piperidinyl, morpholinyl, piperazinyl and tetrahydrofuryl.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein each of R¹ and R² independently from each other are selected from the group of hydrogen; methyl; ethyl; phenyl; and methyl and ethyl, each substituted with a radical selected from the group of phenyl, phenyloxy, dimethylamino, (benzyl)(methyl)amino, (cyclohexylmethyl)(methyl)amino, pyridinyl, pyrrolidinyl, piperidinyl, morpholinyl, piperazinyl and tetrahydrofuryl.

More preferably, R¹ and R² independently from each other are selected from the group of hydrogen and phenyloxyethyl. Most preferably, R¹ is hydrogen and R² is phenyloxyethyl.

In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein
Z¹ and Z² each independently from each other, are CH or N;
X^{A} and X^{B} each independently from each other, are a covalent bond or a C₁₋₄alkyl-radical, wherein one bivalent —CH₂-unit may be replaced by a bivalent phenyl-unit and wherein one or more hydrogen atoms in each moiety X^{A} and X^{B} may be replaced by an oxo radical;
Y^{A} and Y^{B} each independently from each other are a radical selected from the group of t-butyl, NR¹R² and Pir;
R¹ and R² independently from each other are selected from the group of hydrogen alkyl; aryl and alkyl substituted with a radical selected from the group of aryl, aryloxy, Het and —NR^{a}R^{b}, wherein R^{a} and R^{b} each independently are selected from the group of hydrogen, alkyl and arylalkyl;
Pir is a heterocyclic radical selected from the group of pyrrolidinyl piperidinyl; diazepyl; morpholinyl; piperazinyl; 1,2,3,4-tetrahydro-isoquinolinyl; 7,9-diaza-bicyclo[4.2.2]dec-3-enyl and isoindolyl; wherein each Pir-radical may optionally be substituted by one or more radicals selected from the group of hydroxy; oxo; alkyl; alkyloxycarbonyl; aryloxyalkyl; mono-arylaminoalkyl, aryl; arylalkyl; arylalkenyl pyrrolidinyl; furylalkyl, optionally substituted with 1 or 2 alkyl radicals benzofurylalkyl; 2,3-dihydro-benzo[1,4]dioxylalkyl; quinolinylalkyl, benzothienylalkyl and indolylalkyl, optionally substituted with halo.
Het is a monocyclic heterocyclic radical selected from the group of pyridinyl, pyrrolidinyl, piperidinyl, morpholinyl, piperazinyl and tetrahydrofuryl;
   or two adjacent X and Y moieties may form together the bivalent radical 1,2,3,4-tetrahydro-isoquinolinyl, optionally substituted with hydrogen, alkyl, arylalkyl, alkyl-carbonyl, alkylsulphonyl and pyrrolidinylalkyl; and
aryl is naphthalenyl or phenyl, each optionally substituted with 1, 2 or 3 substituents, each independently from each other, selected from the group of halo, cyano, hydroxy and alkyloxy.
   In a further preferred embodiment, the invention relates to a compound according to general Formula (I), a pharmaceutically acceptable acid or base addition salt thereof, a stereochemically isomeric form thereof, an N-oxide form thereof or a quaternary ammonium salt thereof, wherein aryloxyalkyl is phenyloxyethyl, arylalkenyl is 2-methyl-3-phenyl-allyl and isoindolyl is substituted with two oxo-moieties to form an isoindole-1,3-dionyl-moiety.

Most particularly, the invention relates to 4-(4-morpholin-4-ylmethyl-phenyl)-2-[2-(2-phenoxy-ethylamino)-ethyl]-2,4-dihydro-[1,2,4]triazol-3-on, a pharmaceutically acceptable acid or base addition salt thereof, a stereochemically isomeric form thereof, an N-oxide form thereof or a quaternary ammonium salt thereof.

In the framework of this application, alkyl is a straight or branched saturated hydrocarbon radical having from 1 to 6 carbon atoms; or is a cyclic saturated hydrocarbon (cycloalkyl) radical having from 3 to 7 carbon atoms; or is a cyclic saturated hydrocarbon radical having from 3 to 7 carbon atoms attached to a straight or branched saturated hydrocarbon radical having from 1 to 6 carbon atoms; wherein each radical may optionally be substituted on one or more carbon atoms with one or more radicals selected from the group of halo, cyano, hydroxy, amino, oxo, carboxy, nitro, thio and formyl. Preferably, alkyl is methyl, ethyl, propyl, isopropyl, butyl, tert-butyl, cyclopropyl, cyclopentyl, cyclohexyl, cyclohexylmethyl and cyclohexylethyl.

In the framework of this application, alkenyl is an alkyl radical as defined above further having one or more double bonds. Preferably, alkenyl is ethenyl and propenyl.

In the framework of this application, halo is a substituent selected from the group of fluoro, chloro, bromo and iodo and polyhaloalkyl is a straight or branched saturated hydrocarbon radical having from 1 to 6 carbon atoms or a cyclic saturated hydrocarbon radical having from 3 to 7 carbon atoms, wherein one or more carbon atoms is substituted with one or more halo-atoms. Preferably, halo is bromo, fluoro or chloro and preferably, polyhaloalkyl is trifluoromethyl.

In the framework of this application, with “compounds according to the invention” is meant a compound according to the general Formula (I), a pharmaceutically acceptable acid or base addition salt thereof, a stereochemically isomeric form thereof, an N-oxide form thereof or a quaternary ammonium salt thereof.

The pharmaceutically acceptable acid addition salts are defined to comprise the therapeutically active non-toxic acid addition salts forms that the compounds according to Formula (I) are able to form. Said salts can be obtained by treating the base form of the compounds according to Formula (I) with appropriate acids, for example inorganic acids, for example hydrohalic acid, in particular hydrochloric acid, hydrobromic acid, sulphuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-aminosalicylic acid and pamoic acid.

Conversely said acid addition salt forms can be converted into the free base form by treatment with an appropriate base.

The compounds according to Formula (I) containing acidic protons may also be converted into their therapeutically active non-toxic metal or amine addition salts forms (base addition salts) by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said salts forms can be converted into the free forms by treatment with an appropriate acid.

Quaternary ammonium salts of compounds according to Formula (I) defines said compounds which are able to form by a reaction between a basic nitrogen of a compound according to Formula (I) and an appropriate quaternizing agent, such as, for example, an optionally substituted alkylhalide, arylhalide or arylalkylhalide, in particular methyliodide and benzyliodide. Other reactants with good leaving groups may also be used, such as, for example, alkyl trifluoromethanesulfonates, alkyl methanesulfonates and alkyl p-toluenesulfonates. A quaternary ammonium salt has a positively charged nitrogen. Pharmaceutically acceptable counterions include chloro, bromo, iodo, trifluoroacetate and acetate ions.

The term addition salt as used in the framework of this application also comprises the solvates that the compounds according to Formula (I) as well as the salts thereof, are able to form. Such solvates are, for example, hydrates and alcoholates.

The N-oxide forms of the compounds according to Formula (I) are meant to comprise those compounds of Formula (I) wherein one or several nitrogen atoms are oxidized to the so-called N-oxide, particularly those N-oxides wherein one or more tertiary nitrogens (e.g. of the piperazinyl or piperidinyl radical) are N-oxidized. Such N-oxides can easily be obtained by a skilled person without any inventive skills and they are obvious alternatives for the compounds according to Formula (I) since these compounds are metabolites, which are formed by oxidation in the human body upon uptake. As is generally known, oxidation is normally the first step involved in drug metabolism (Textbook of Organic Medicinal and Pharmaceutical Chemistry, 1977, pages 70-75). As is also generally known, the metabolite form of a compound can also be administered to a human instead of the compound per se, with much the same effects.

The compounds of Formula (I) may be converted to the corresponding N-oxide forms following art-known procedures for converting a trivalent nitrogen into its N-oxide form. Said N-oxidation reaction may generally be carried out by reacting the starting material of Formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

The term “stereochemically isomeric forms” as used hereinbefore defines all the possible isomeric forms that the compounds of Formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R- or S-configuration; substituents on bivalent cyclic (partially) saturated radicals may have either the cis- or trans-configuration. Compounds encompassing double bonds can have an E or Z-stereochemistry at said double bond. Stereochemically isomeric forms of the compounds of Formula (I) are obviously intended to be embraced within the scope of this invention.

Following CAS nomenclature conventions, when two stereogenic centers of known absolute configuration are present in a molecule, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where R*is always specified as the reference center and [R*,R*] indicates centers with the same chirality and [R*,S*] indicates centers of unlike chirality. For example, if the lowest-numbered chiral center in the molecule has an S configuration and the second center is R, the stereo descriptor would be specified as S—[R*,S*]. If “α” and “β” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system (hydrogen atom in compounds according to Formula (I)) relative to the position of the highest priority substituent on the reference atom is denominated “α”, if it is on the same side of the mean plane determined by the ring system, or “β”, if it is on the other side of the mean plane determined by the ring system.

The invention also comprises derivative compounds (usually called “pro-drugs”) of the pharmacologically-active compounds according to the invention, which are degraded in vivo to yield the compounds according to the invention. Pro-drugs are usually (but not always) of lower potency at the target receptor than the compounds to which they are degraded. Pro-drugs are particularly useful when the desired compound has chemical or physical properties that make its administration difficult or inefficient. For example, the desired compound may be only poorly soluble, it may be poorly transported across the mucosal epithelium, or it may have an undesirably short plasma half-life. Further discussion on pro-drugs may be found in Stella, V. J. et al., “Prodrugs”, Drug Delivery Systems, 1985, pp. 112-176, and Drugs, 1985, 29, pp. 455-473.

Pro-drugs forms of the pharmacologically-active compounds according to the invention will generally be compounds according to Formula (I), the pharmaceutically acceptable acid or base addition salts thereof, the stereochemically isomeric forms thereof and the N-oxide form thereof, having an acid group which is esterified or amidated. Included in such esterified acid groups are groups of the formula —COOR^{x}, where R^{x} is a C₁₋₆alkyl, phenyl, benzyl or one of the following groups:

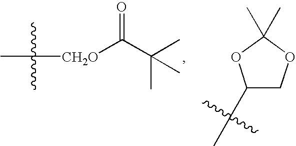

Amidated groups include groups of the formula —CONR^{y}R^{z}, wherein R^{y} is H, C₁₋₆alkyl, phenyl or benzyl and R^{z} is —OH, H, C₁₋₆alkyl, phenyl or benzyl. Compounds according to the invention having an amino group may be derivatised with a ketone or an aldehyde such as formaldehyde to form a Mannich base. This base will hydrolyze with first order kinetics in aqueous solution.

In the framework of this application, with “compounds according to the invention” is meant a compound according to the general Formula (I), the pharmaceutically acceptable acid or base addition salts thereof, the stereochemically isomeric forms thereof, the N-oxide form thereof and a prodrug thereof.

In the framework of this application, an element, in particular when mentioned in relation to a compound according to Formula (I), comprises all isotopes and isotopic mixtures of this element, either naturally occurring or synthetically produced, either with natural abundance or in an isotopically enriched form. In particular, when hydrogen is mentioned, it is understood to refer to ¹H, ²H, ³H and mixtures thereof when carbon is mentioned, it is understood to refer to ¹¹C, ¹²C, ¹³C, ¹⁴C and mixtures thereof; when nitrogen is mentioned, it is understood to refer to ¹³N, ¹⁴N, ¹⁵N and mixtures thereof; when oxygen is mentioned, it is understood to refer to ¹⁴O, ¹⁵O, ¹⁶O, ¹⁷O, ¹⁸O and mixtures thereof; and when fluor is mentioned, it is understood to refer to ¹⁸F, ¹⁹F and mixtures thereof.

The compounds according to the invention therefore also comprise compounds with one or more isotopes of one or more element, and mixtures thereof, including radioactive compounds, also called radiolabelled compounds, wherein one or more non-radioactive atoms has been replaced by one of its radioactive isotopes. By the term “radiolabelled compound” is meant any compound according to Formula (I), an N-oxide form, a pharmaceutically acceptable addition salt or a stereochemically isomeric form thereof, which contains at least one radioactive atom. For example, compounds can be labelled with positron or with gamma emitting radioactive isotopes. For radioligand-binding techniques (membrane receptor assay), the ³H-atom or the ¹²⁵I-atom is the atom of choice to be replaced. For imaging, the most commonly used positron emitting (PET) radioactive isotopes are ¹¹C, ¹⁸F, ¹⁵O and ¹³N, all of which are accelerator produced and have half-lives of 20, 100, 2 and 10 minutes respectively. Since the half-lives of these radioactive isotopes are so short, it is only feasible to use them at institutions which have an accelerator on site for their production, thus limiting their use. The most widely used of these are ¹⁸F, ^{99m}Tc, ²⁰¹Tl and ¹²³I. The handling of these radioactive isotopes, their production, isolation and incorporation in a molecule are known to the skilled person.

In particular, the radioactive atom is selected from the group of hydrogen, carbon, nitrogen, sulfur, oxygen and halogen. Preferably, the radioactive atom is selected from the group of hydrogen, carbon and halogen.

In particular, the radioactive isotope is selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

### Preparation

The compounds according to the invention can generally be prepared by a succession of steps, each of which is known to the skilled person. In particular, the triazolone, tetrazolone and imidazole derivatives can be prepared according to the following synthesis methods.

### Synthesis of Triazolone Derivatives

### Method A

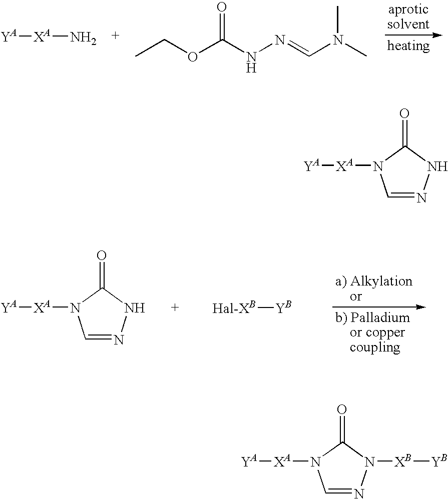

Reactions of the starting amino or anilino derivatives with [(dimethylamino)methylene]-hydrazinecarboxylic acid ethyl ester can be performed in a polar aprotic solvent, such as acetonitrile, at a convenient temperature, either by conventional heating or under microwave irradiation, for a period of time to ensure the completion of the reaction, typically 20 minutes at 180° C. under microwave irradiation.

The Hal-radical in Hal-X^{B}-Y^{B} means a halogen atom, such as Cl, Br or I. The alkylation reactions a) can be carried out in an aprotic polar solvent, such as for instance acetonitrile, DMF or dioxane; in the presence of an inorganic or organic base, such as K₂CO₃, Na₂CO₃, Cs₂CO₃, NaH, Et₃N, BTPP or PS-TBD; at a convenient temperature, either under microwave irradiation or by conventional heating.

When X^{B} is an aryl ring, Hal is a Br or I atom or a suitable group, such as CF₃SO₃, and the Palladium coupling reaction b) is performed in an aprotic solvent such as toluene or dioxane; in the presence of a Palladium catalyst such as Pd(AcO)₂ or Pd(dba)₃; in the presence of a base such as Cs₂CO₃ or t-BuONa and of a ligand, such as BINAP or Xantphos; at a convenient temperature, either by conventional heating or under microwave irradiation, for a period of time to ensure the completion of the reaction. As an alternative, a copper coupling reaction can be also used to prepare the aryl derivatives. In the latter case Hal is a Br or I atom or a suitable group, such as B(OH)₂. The reaction is performed in aprotic solvents such as dichloroethane, in the presence of a copper compound, such as Cu(AcO)₂, either in catalytic or equivalent amount; in the presence of a suitable ligand, such as pyridine and at a convenient temperature, either by conventional heating or under microwave irradiation, for a period of time to ensure the completion of the reaction. Additionally, an inorganic base such as K₃PO₄ can be added to the reaction.

### Method B

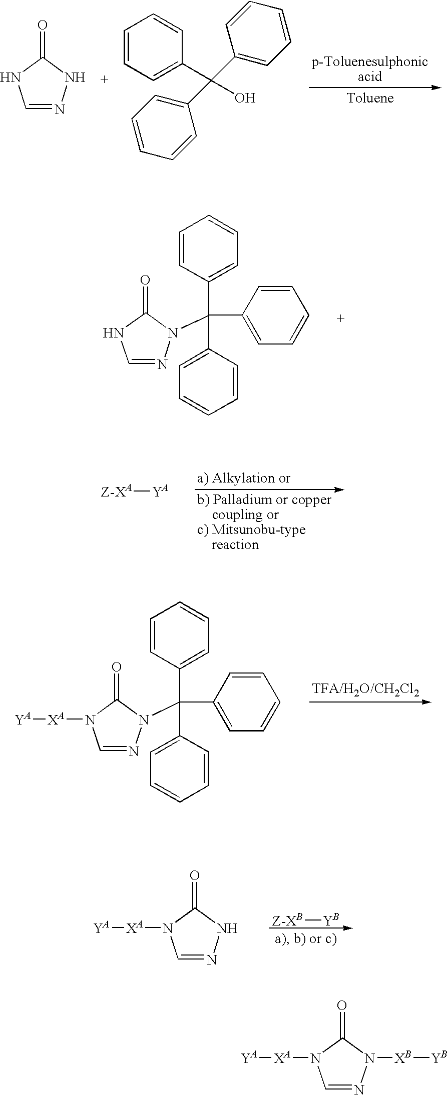

In the above scheme Z is a halogen atom as in Method A or a hydroxyl group. Alkylation reactions a) and the Palladium or copper coupling reactions b) can be performed in the same way as described for Method A. In the case where Z=OH a Mitsunobu-type reaction can be used to obtain the desired compounds. Thus, the corresponding alcohol can be reacted with the required triazolone intermediate, in the presence of either di-tert-butylazadicarboxylate, DEAD or DIAD, and triphenylphosphine optionally supported on polymer, in an aprotic solvent such as dichloromethane.

### Synthesis of Tetrazolone Derivatives

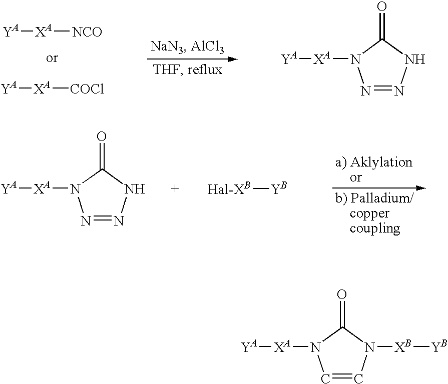

The desired tetrazolone intermediate shown above, can be obtained by any one of the two methods described in literature (Biorg. Med. Chem. Lett. 1999, 1251-1254 and J. Med. Chem. 1986, 29, 2290-2297). Subsequent alkylation or palladium/copper coupling reactions with the required intermediates can be carried out under the same reaction conditions as those described in the scheme shown for Triazolones-Method A.

### Synthesis of Imidazolone Derivatives

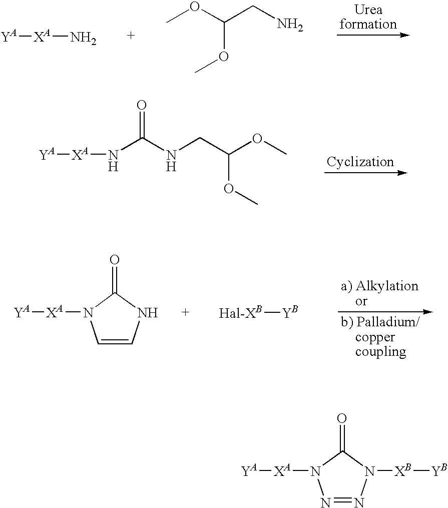

The desired imidazolone intermediate can be obtained in three steps using methodologies very well known by anyone skilled in the art. Substitution of both imidazoles of CDI by heating the reagents for a period of time to ensure the completion of the reaction, in a suitable aprotic solvent, such as ethyl acetate or acetonitrile. The intermediate can be further cyclizated by heating the product at a convenient temperature, either by conventional heating or under microwave irradiation, under aqueous acidic conditions, such as a 10% solution of hydrochloric acid in water. Subsequent alkylation or a palladium/copper coupling reaction with the required intermediates can be carried out under the same reaction conditions as those described in the scheme shown for Triazolones-Method A.

The intermediary compound according to Formula (I) wherein at least one of Y^{A} and Y^{B} is a radical selected from the group of alkyl; halo; formyl; amino; morpholinyl; alkylSO₃—; cyano; hydroxy; alkyloxycarbonyl, in particular ethyloxycarbonyl and t-butyloxycarbonyl (t-BOC); arylalkyloxycarbonyl, in particular benzyloxycarbonyl and alkyloxy, in particular methoxy also form part of the invention.

Transformations of different Y^{A} or Y^{B} groups, present in the intermediate compounds, into different Y^{A} and Y^{B} groups present in final compounds according to Formula (I) can be performed by synthetic methods well known by everyone skilled in the art.

### Pharmacology

The compounds according to the invention, in particular compounds according to Formula (I), the pharmaceutically acceptable acid or base addition salts thereof, a stereochemically isomeric form thereof, an N-oxide form thereof or a quaternary ammonium salt thereof, have surprisingly been shown to have a binding affinity towards α₂-adrenergic receptor, in particular towards α_{2C}-adrenergic receptor, in particular as an antagonist.

In view of their above mentioned potency, the compounds according to the invention are suitable for the prevention and/or treatment of diseases where antagonism of the α₂-adrenergic receptor, in particular antagonism of the α_{2C}-adrenergic receptor is of therapeutic use. In particular, the compounds according to the invention may be suitable for treatment and/or prophylaxis in the following diseases
Central nervous system disorders, including:
Mood disorders, including particularly major depressive disorder, depression with or without psychotic features, catatonic features, melancholic features, atypical features of postpartum onset and, in the case of recurrent episodes, with or without seasonal pattern, dysthymic disorder, bipolar I disorder, bipolar II disorder, cyclothymic disorder, recurrent brief depressive disorder, mixed affective disorder, bipolar disorder not otherwise specified, mood disorder due to a general medical condition, substance-induced mood disorder, mood disorder not otherwise specified, seasonal affective disorder and premenstrual dysphoric disorders.
Anxiety disorders, including panic attack, agoraphobia, panic disorder without agoraphobia, agoraphobia without history of panic disorder, specific phobia, social phobia, obsessive-compulsive disorder, posttraumatic stress disorder, acute stress disorder, generalized anxiety disorder, anxiety disorder due to a general medical condition, substance-induced anxiety disorder and anxiety disorder not otherwise specified.
Stress-related disorders associated with depression and/or anxiety, including acute stress reaction, adjustment disorders (brief depressive reaction, prolonged depressive reaction, mixed anxiety and depressive reaction, adjustment disorder with predominant disturbance of other emotions, adjustment disorder with predominant disturbance of conduct, adjustment disorder with mixed disturbance of emotions and conduct, adjustment disorders with other specified predominant symptoms) and other reactions to severe stress.
Dementia, amnesic disorders and cognitive disorders not otherwise specified, especially dementia caused by degenerative disorders, lesions, trauma, infections, vascular disorders, toxins, anoxia, vitamin deficiency or endocrinic disorders, or amnesic disorders caused by alcohol or other causes of thiamine deficiency, bilateral temporal lobe damage due to Herpes simplex encephalitis and other limbic encephalitis, neuronal loss secondary to anoxia/hypoglycaemia/severe convulsions and surgery, degenerative disorders, vascular disorders or pathology around ventricle III.
Cognitive disorders, in particular due to cognitive impairment resulting from other medical conditions.
Personality disorders, including paranoid personality disorder, schizoid personality disorder, schizotypical personality disorder, antisocial personality disorder, borderline personality disorder, histrionic personality disorder, narcissistic personality disorder, avoidant personality disorder, dependent personality disorder, obsessive-compulsive personality disorder and personality disorder not otherwise specified.
Schizoaffective disorders resulting from various causes, including schizoaffective disorders of the manic type, of the depressive type, of mixed type, paranoid, disorganized, catatonic, undifferentiated and residual schizophrenia, schizophreniform disorder, schizoaffective disorder, delusional disorder, brief psychotic disorder, shared psychotic disorder, substance-induced psychotic disorder and psychotic disorder not otherwise specified.
Akinesia, akinetic-rigid syndromes, dyskinesia and medication-induced parkinsonism, Gilles de la Tourette syndrome and its symptoms, tremor, chorea, myoclonus, tics and dystonia.
Attention-deficit/hyperactivity disorder (ADHD).
Parkinson's disease, drug-induced Parkinsonism, post-encephalitic Parkinsonism, progressive supranuclear palsy, multiple system atrophy, corticobasal degeneration, parkinsonism-ALS dementia complex and basal ganglia calcification.
Dementia of the Alzheimer's type, with early or late onset, with depressed mood.
Behavioural disturbances and conduct disorders in dementia and the mentally retarded, including restlessness and agitation.
Extra-pyramidal movement disorders.
Down's syndrome.
Akathisia.
Eating Disorders, including anorexia nervosa, atypical anorexia nervosa, bulimia nervosa, atypical bulimia nervosa, overeating associated with other psychological disturbances, vomiting associated with other psychological disturbances and non-specified eating disorders.
AIDS-associated dementia.
Chronic pain conditions, including neuropathic pain, inflammatory pain, cancer pain and post-operative pain following surgery, including dental surgery. These indications might also include acute pain, skeletal muscle pain, low back pain, upper extremity pain, fibromyalgia and myofascial pain syndromes, orofascial pain, abdominal pain, phantom pain, tic douloureux and atypical face pain, nerve root damage and arachnoiditis, geriatric pain, central pain and inflammatory pain.
Neurodegenerative diseases, including Alzheimer's disease, Huntington's chorea, Creutzfeld-Jacob disease, Pick's disease, demyelinating disorders, such as multiple sclerosis and ALS, other neuropathies and neuralgia, multiple sclerosis, amyotropical lateral sclerosis, stroke and head trauma.
Addiction disorders, including:
Substance dependence or abuse with or without physiological dependence, particularly where the substance is alcohol, amphetamines, amphetamine-like substances, caffeine, cannabis, cocaine, hallucinogens, inhalants, nicotine, opioids, phencyclidine, phencyclidine-like compounds, sedative-hypnotics, benzodiazepines and/or other substances, particularly useful for treating withdrawal from the above substances and alcohol withdrawal delirium.
Mood disorders induced particularly by alcohol, amphetamines, caffeine, cannabis, cocaine, hallucinogens, inhalants, nicotine, opioids, phencyclidine, sedatives, hypnotics, anxiolitics and other substances.
Anxiety disorders induced particularly by alcohol, amphetamines, caffeine, cannabis, cocaine, hallucinogens, inhalants, nicotine, opioids, phencyclidine, sedatives, hypnotics, anxiolitics and other substances and adjustment disorders with anxiety.
Smoking cessation.
Body weight control, including obesity.
Sleep disorders and disturbances, including:
Dyssomnias and/or parasomnias as primary sleep disorders, sleep disorders related to another mental disorder, sleep disorder due to a general medical condition and substance-induced sleep disorder.
Circadian rhythms disorders.
Improving the quality of sleep.
Sexual dysfunction, including sexual desire disorders, sexual arousal disorders, orgasmic disorders, sexual pain disorders, sexual dysfunction due to a general medical condition, substance-induced sexual dysfunction and sexual dysfunction not otherwise specified.

The invention therefore relates to a compound according to the general Formula (I), a pharmaceutically acceptable acid or base addition salt thereof, a stereochemically isomeric form thereof, an N-oxide form thereof or a quaternary ammonium salt thereof, for use as a medicine.

The invention also relates to the use of a compound according to the invention for the preparation of a medicament for the prevention and/or treatment of central nervous system disorders, mood disorders, anxiety disorders, stress-related disorders associated with depression and/or anxiety, cognitive disorders, personality disorders, schizoaffective disorders, Parkinson's disease, dementia of the Alzheimer's type, chronic pain conditions, neurodegenerative diseases, addiction disorders, mood disorders and sexual dysfunction.

The compounds according to the invention may be co-administered as add-on treatment and/or prophylaxis in the above listed diseases in combination with antidepressants, anxiolytics and/or antipsychotics which are currently available or in development or which will become available in the future, in particular to improve efficacy and/or onset of action. It will be appreciated that the compounds of the present invention and the other agents may be present as a combined preparation for simultaneous, separate or sequential use for the prevention and/or treatment of depression and/or anxiety. Such combined preparations may be, for example, in the form of a twin pack. It will also be appreciated that the compounds of the present invention and the other agents may be administered as separate pharmaceutical compositions, either simultaneously or sequentially.

The invention therefore relates to the use of the compounds according to the invention as an add-on treatment in combination with one or more other compounds selected from the group of antidepressants, anxiolytics and antipsychotics.

Suitable classes of antidepressant agents include norepinephrine reuptake inhibitors, selective serotonin reuptake inhibitors (SSRI's), monoamine oxidase inhibitors (MAOI's), reversible inhibitors of monoamine oxidase (RIMA's), serotonin and noradrenaline reuptake inhibitors (SNRI's), noradrenergic and specific serotonergic antidepressants (NaSSA's), corticotropin releasing factor (CRF) antagonists, α-adrenoreceptor antagonists and atypical antidepressants.

Suitable examples of norepinephrine reuptake inhibitors include amitriptyline, clomipramine, doxepin, imipramine, trimipramine, amoxapine, desipramine, maprotiline, nortriptyline, protriptyline, reboxetine and pharmaceutically acceptable salts thereof.

Suitable examples of selective serotonin reuptake inhibitors include fluoxetine, fluvoxamine, paroxetine, sertraline and pharmaceutically acceptable salts thereof.

Suitable examples of monoamine oxidase inhibitors include isocarboxazid, phenelzine, tranylcypromine, selegiline and pharmaceutically acceptable salts thereof.

Suitable examples of reversible inhibitors of monoamine oxidase include moclobemide and pharmaceutically acceptable salts thereof.

Suitable examples of serotonin and noradrenaline reuptake inhibitors include venlafaxine and pharmaceutically acceptable salts thereof.

Suitable atypical antidepressants include bupropion, lithium, nefazodone, trazodone, viloxazine, sibutramine and pharmaceutically acceptable salts thereof.

Other suitable antidepressants include adinazolam, alaproclate, amineptine, amitriptyline/chlordiazepoxide combination, atipamezole, azamianserin, bazinaprine, befuraline, bifemelane, binodaline, bipenamol, brofaromine, bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, monirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine and zometapine and pharmaceutically acceptable salts thereof, and St. John's wort herb, or Hypericum perforatum, or extracts thereof.

Suitable classes of anti-anxiety agents include benzodiazepines and 5-HT_{1A} receptor agonists or antagonists, especially 5-HT_{1A} partial agonists, corticotropin releasing factor (CRF) antagonists, compounds having muscarinic cholinergic activity and compounds acting on ion channels. In addition to benzodiazepines, other suitable classes of anti-anxiety agents are nonbenzodiazepine sedative-hypnotic drugxs such as zolpidem; mood-stabilizing drugs such as clobazam, gabapentin, lamotrigine, loreclezole, oxcarbamazepine, stiripentol and vigabatrin; and barbiturates.

Suitable antipsychotic agents are selected from the group consisting of acetophenazine, in particular the maleate salt; alentemol, in particular the hydrobromide salt; alpertine; azaperone; batelapine, in particular the maleate salt; benperidol; benzindopyrine, in particular the hydrochloride salt; brofoxine; bromperidol; butaclamol, in particular the hydrochloride salt; butaperazine; carphenazine, in particular the maleate salt; carvotroline, in particular the hydrochloride salt; chlorpromazine; chlorprothixene; cinperene; cintriamide; clomacran, in particular the phosphate salt; clopenthixol; clopimozide; clopipazan, in particular the mesylate salt; cloroperone, in particular the hydrochloride salt; clothiapine; clothixamide, in particular the maleate salt; clozapine; cyclophenazine, in particular the hydrochloride salt; droperidol; etazolate, in particular the hydrochloride salt; fenimide; flucindole; flumezapine; fluphenazine, in particular the decanoate, enanthate and/or hydrochloride salts; fluspiperone; fluspirilene; flutroline; gevotroline, in particular the hydrochloride salt; halopemide; haloperidol; iloperidone; imidoline, in particular the hydrochloride salt; lenperone; loxapine; mazapertine, in particular the succinate salt; mesoridazine; metiapine; milenperone; milipertine; molindone, in particular the hydrochloride salt; naranol, in particular the hydrochloride salt; neflumozide, in particular the hydrochloride salt; ocaperidone; olanzapine; oxiperomide; penfluridol; pentiapine, in particular the maleate salt; perphenazine; pimozide; pinoxepin, in particular the hydrochloride salt; pipamperone; piperacetazine; pipotiazine, in particular the palmitate salt; piquindone, in particular the hydrochloride salt; prochlorperazine, in particular the edisylate salt; prochlorperazine, in particular the maleate salt; promazine, in particular the hydrochloride salt; quetiapine; remoxipride; risperidone; rimcazol, in particular the hydrochloride salt; seperidol, in particular the hydrochloride salt; sertindole; setoperone; spiperone; sulpiride; thioridazine; thiothixene; thorazine; tioperidone, in particular the hydrochloride salt; tiospirone, in particular the hydrochloride salt; trifluoperazine, in particular the hydrochloride salt; trifluperidol; triflupromazine; ziprasidone, in particular the hydrochloride salt; and mixtures thereof.

### Pharmaceutical Compositions

The invention also relates to a pharmaceutical composition comprising a pharmaceutically acceptable carrier or diluent and, as active ingredient, a therapeutically effective amount of a compound according to the invention, in particular a compound according to Formula (I), a pharmaceutically acceptable acid or base addition salt thereof, a stereochemically isomeric form thereof, an N-oxide form thereof or a quaternary ammonium salt thereof.

The compounds according to the invention, in particular the compounds according to Formula (I), the pharmaceutically acceptable acid or base addition salt thereof, a stereochemically isomeric form thereof, an N-oxide form thereof or a quaternary ammonium salt thereof, or any subgroup or combination thereof may be formulated into various pharmaceutical forms for administration purposes. As appropriate compositions there may be cited all compositions usually employed for systemically administering drugs.

To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally, rectally, percutaneously, by parenteral injection or by inhalation. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. examples of such unit dosage forms are tablets (including scored or coated tablets), capsules, pills, powder packets, wafers, suppositories, injectable solutions or suspensions and the like, and segregated multiples thereof. Since the compounds according to the invention are potent orally administrable dopamine antagonists, pharmaceutical compositions comprising said compounds for administration orally are especially advantageous.

The invention also relates to a pharmaceutical composition comprising the compounds according to the invention and one or more other compounds selected from the group of antidepressants, anxiolytics and antipsychotics as well as to the use of such a composition for the manufacture of a medicament, in particular to improve efficacy and/or onset of action in the treatment of depression and/or anxiety.

The following examples are intended to illustrate but not to limit the scope of the present invention.

### Experimental Part

### A. Preparation of the Intermediate Compounds

Hereinafter “RT” means room temperature, “CDI” means 1,1′-carbonyldiimidazole, “DIPE” means diisopropylether, “MIK” means methyl isobutyl ketone, “BINAP” means [1,1′-binaphthalene]-2,2′-diylbis[diphenylphosphine], “NMP” means 1-methyl-2-pyrrolidinone, “Pd₂(dba)₃” means tris(dibenzylideneacetone)dipalladium, “BTTP” means 1,1′,1″-[(1,1-dimethylethyl)phosphinimylidyne]tris-pyrrolidine, “Xantphos” means (9,9-dimethyl-9H-xanthene-4,5-diyl)bis[diphenyl-phosphine, “HATU” means 1-[bis(dimethylamino)methylene]-1H-1,2,3-triazolo[4,5-b]pyridinium 3-oxide, hexa-fluorophosphate and “DMF” means N,N-dimethylformamide.

### Example A1

### a-1. Preparation of Intermediate Compound 1

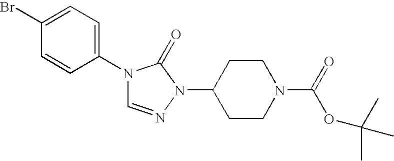

A mixture of 4-(4-bromophenyl)-2,4-dihydro-3H-1,2,4-triazol-3-one (0.002 mol), 1,1-dimethylethyl ester of 4-iodo-1-piperidine carboxylic acid (0.003 mol) and Cs₂CO₃ (0.004 mol) in CH₃CN (7 ml) was heated for 10 minutes under microwave conditions at 150° C. and then for 10 minutes at 180° C. The resulting solids were filtered off and the solvent was evaporated. The residue was purified over a filter using CH₂Cl₂ and then CH₂Cl₂/CH₃OH (98/2) and the desired product was collected. Yield: 0.25 g of intermediate compound 1 (30%).

### b. Preparation of Intermediate Compound 2

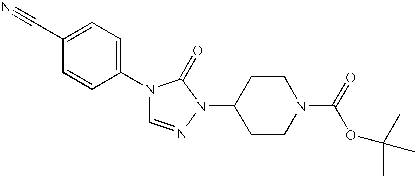

A mixture of intermediate compound 1 (0.00106 mol), zink cyanide (0.00064 mol) and Pd(PPh₃)₄ (0.000088 mol) in deoxygenated DMF (5 ml) was reacted for 20 minutes under microwave conditions at 150° C., then the solids were filtered off and the solvent was evaporated. The residue was purified by column chromatography on silica gel using CH₂Cl₂/CH₃OH (98/2) and CH₂Cl₂/CH₃OH (96/4) and then the desired product was collected. Yield: 0.235 g of intermediate compound 2 (60%).

### c. Preparation of Intermediate Compound 3 (Removal of t-BOC)

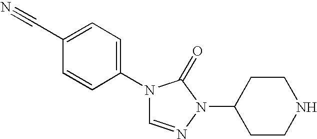

A mixture of intermediate compound 2 (0.00064 mol) in trifluoroacetic acid (0.5 ml) and CH₂Cl₂ (2 ml) was stirred for 1 hour at room temperature and a saturated Na₂CO₃ solution was added. The organic layer was separated, dried (Na₂SO₄), filtered off and the solvent was evaporated. Yield: 0.124 g of intermediate compound 3 (72%).

### d. Preparation of Intermediate Compound 4

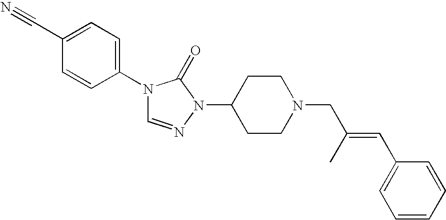

A mixture of intermediate compound 3 (prepared according to A1.c) (0.00046 mol), 2-methyl-3-phenyl-2-propenal (0.00092 mol) and NaBH(OAc)₃ (0.00092 mol) in 1,2-dichloroethane (3 ml) was heated for 10 minutes under microwave conditions at 100° C. and then a saturated NH₄Cl solution was added. The organic layer was separated, dried (Na₂SO₄), filtered off and the solvent was evaporated. The residue was purified in a manifold (vacuum) (eluent gradient: CH₂Cl₂/CH₃OH 98/2, 96/4) and then further purified by catch and release. The product fractions were collected and the solvent was evaporated. Yield: 0.014 g of intermediate compound 4 (8%).

### e. Preparation of Intermediate Compound 5

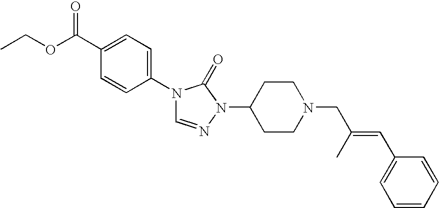

A mixture of intermediate compound 4 (0.0005 mol) in trifluoromethane sulfonic acid anhydride (1 ml) and ethanol (4 ml) was stirred and refluxed for 48 hours, then the reaction mixture was poured out into a saturated Na₂CO₃ solution (10 ml) and extracted with CH₂Cl₂. The organic layer was separated, dried (Na₂SO₄), filtered off and the solvent was evaporated. The residue was percolated using a manifold (eluent gradient: CH₂Cl₂/EtOAc 4/1, 1/1). The product fractions were collected and the solvent was evaporated. Yield: 0.079 g of intermediate compound 5 (35%).

### f. Preparation of Intermediate Compound 6

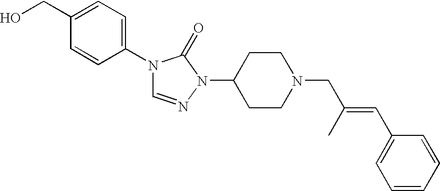

DIBAL (0.00045 mol; 1.0 M in toluene) was added at −78° C. to a mixture of intermediate compound 5 (0.00018 mol) in dry toluene (2 ml), then the reaction mixture was allowed to reach room temperature and was stirred for 30 minutes at room temperature. CH₂Cl₂/CH₃OH (1/1) was added and the excess of starting material DIBAL-H was quenched with a saturated NH₄Cl solution (0.5 ml). The resulting solids were filtered over dicalite and then the organic filtrate was dried (Na₂SO₄) and the solvent was evaporated. Yield: 0.078 g of intermediate compound 6 (100%, used as such in the next reaction step without further purification).

### Example A2

### a. Preparation of Intermediate Compound 27

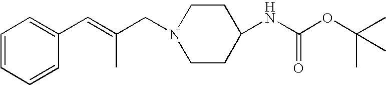

A mixture of 1,1-dimethylethyl-4-piperidinyl carbamic acid ester (0.0349 mol), 2-methyl-3-phenyl-2-propenal (0.0268 mol) and NaBH(Oac)₃ (0.0349 mol) in dichloroethane (130 ml) and molecular sieves (4 Å) (q.s.) was stirred overnight at room temperature, then the reaction mixture was filtered over celite. The filtrate was treated with a 10% NH₄Cl solution and extracted with EtOAc. The organic layer was separated, dried (Na₂SO₄), filtered and the solvent was evaporated dry. Yield: 11.44 g of intermediate compound 27 (99%).

### b. Preparation of Intermediate Compound 28

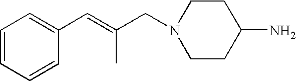

Trifluoroacetic acid (23.6 ml) was added dropwise to a solution of intermediate compound 27 (0.014 mol) in CH₂Cl₂ (100 ml) cooled on ice-water bath. The reaction mixture was stirred from 0° C. to room temperature for 2 hours, then alkalised with a 50% NaOH solution and extracted. The organic layer was separated, dried (Na₂SO₄), filtered and the solvent was evaporated dry. Yield: 2.72 g of intermediate compound 28 (84%).

### c. Preparation of Intermediate Compound 7

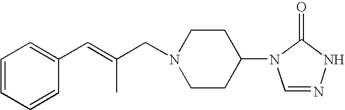

A mixture of intermediate compound 28 (0.005 mol) and [(dimethylamino)methylene]-hydrazinecarboxylic acid ethyl ester (0.010 mol) in CH₃CN (20 ml) was heated in a microwave oven for 20 minutes at 180° C., then the solvent was evaporated and the obtained residue was washed with diethyl ether. Yield: 1.400 g of intermediate compound 7 (94%).

### d. Preparation of Intermediate Compound 8

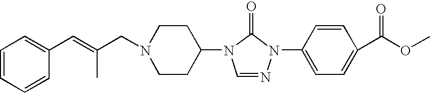

A mixture of intermediate compound 7 (0.00084 mol), 4-bromobenzoic acid methyl ester (0.00126 mol), Pd₂(dba)₃ (0.000042 mol), BINAP (0.000126 mol) and t-BuONa (0.00126 mol) in deoxygenated toluene (3 ml) was heated in a microwave oven for 15 minutes at 175° C. and then CH₂Cl₂ and a 10% NH₄Cl solution were added. The organic layer was separated, dried (Na₂SO₄), filtered off and the solvent was evaporated. The residue was purified in a manifold (vac.) (eluent gradient: CH₂Cl₂/EtOAc 4/1, 2/1). The product fractions were collected and the solvent was evaporated. Yield: 0.107 g of intermediate compound 8 (29%).

### Example A3

### Preparation of Intermediate Compound 9

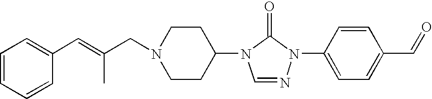

A mixture of

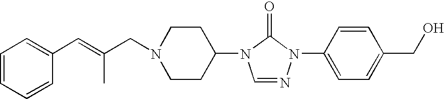

(prepared according to A1.f) (0.00010 mol) and MnO₂ (0.00050 mol) in dichloroethane (1 ml) was heated in a microwave oven for 10 minutes at 120° C. The solids were filtered over dicalite and washed with CH₂Cl₂, then the solvent was evaporated. Yield: 0.037 g of intermediate compound 9 (92%).

### Example A4

### a. Preparation of Intermediate Compound 13

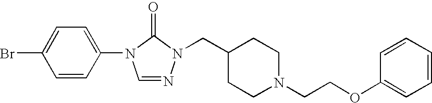

A mixture of intermediate compound

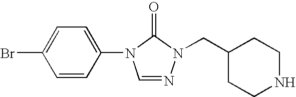

(prepared according to A1.c) (0.0015 mol), (2-bromoethoxy)benzene (0.00165 mol) and K₂CO₃ (0.0030 mol) in CH₃CN (10 ml) was heated under microwave irradiation for 10 minutes at 120° C. and then CH₂Cl₂ was added. The resulting solids were filtered off and the solvent was evaporated. Finally, the obtained residue was washed with ethyl ether. Yield: 0.392 g of intermediate compound 13 (57%).

### b. Preparation of Intermediate Compound 10

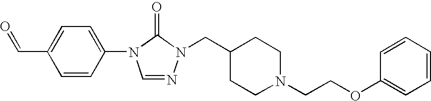

CO (gas) was bubbled through a mixture of intermediate compound 13 (prepared according to A4.a) (0.00022 mol), NaHCO₂ (0.00033 mol) and Cl₂Pd(PPh₃)₂ (0.000009 mol) in DMF (5 ml), then the reaction mixture was heated to 110° C. with CO (gas) still bubbling through. Extra NaHCO₂ (2×q.s.) and Cl₂Pd(PPh₃)₂ (2×q.s.) were added and the mixture was heated for 2 hours at 110° C. with CO (gas) bubbling through. The solvent was evaporated and the residue was taken up in CH₂Cl₂. The solids were filtered off over dicalite and the solvent was evaporated. The obtained residue was purified in a manifold (vacuum) (eluent: CH₂Cl₂/CH₃OH 96/4). The product fractions were collected and the solvent was evaporated. Yield: 0.081 g of intermediate compound 10 (91%).

### Example A5

### a. Preparation of Intermediate Compound 11

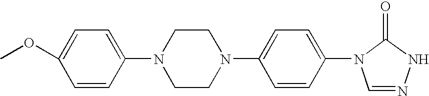

A mixture of N-[4-[4-(4-methoxyphenyl)-1-piperazinyl]phenyl]hydrazinecarboxamide (prepared according to the teachings in WO94/18978 of which the content is included herein) (0.001 mol) and methaneimidamide (0.029 mol) in DMSO (10 ml) was heated for 2 hours at 160° C. After cooling, the reaction mixture was poured into a mixture of MIK and DIPE. The precipitate was filtered off and treated with activated charcoal in DMF. After filtration, the product was allowed to crystallize. The product was filtered off and dried. Yielding: 1 g of intermediate compound 11 (28%).

### b. Preparation of Intermediate Compound 12

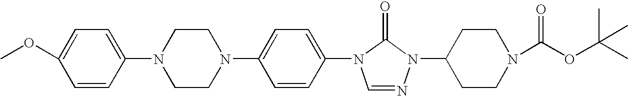

A mixture of intermediate compound 11 (prepared according to A5.a) (0.001 mol), 4-iodo-1-piperidine carboxylic acid 1,1-dimethylethyl ester (0.002 mol) and BTPP (0.002 mol) in CH₃CN (3.5 ml) was heated for 20 minutes at 120° C. under microwave irradiation and the collected solids were washed with CH₃CN, then purified by short open column chromatography (eluent 1: CH₂Cl₂/EtOAc 4/1, 1/1; eluent 2: CH₂Cl₂/2-propanone 1/1). The product fractions were collected and the solvent was evaporated. Yield: 0.120 g of intermediate compound 12 (22%)

### Example A6

### a. Preparation of Intermediate Compound 26

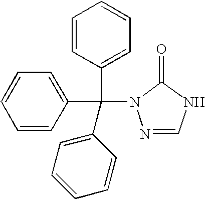

To a mixture of 1,2-dihydro-3H-1,2,4-triazol-3-one (5.74 mmol) in toluene (70 ml), alpha,alpha-diphenylbenzenemethanol (4.7 mmol) and p-toluenesulphonic acid (1.91 mmol) were added. The reaction was heated at reflux using a Dean-Stark separator under Nitrogen atmosphere for 20 hours. The solution was cooled and quenched with 2% of an aqueous solution of NaHCO₃ and extracted with CH₂Cl₂ (3×100 ml). The organic layer was separated, dried (Na₂SO₄) and the solvent evaporated. The residue was purified by column chromatography (eluent: CH₂Cl₂/MeOH 9/1). Yield: 925 mg of intermediate compound 26 (60%).

### b. Preparation of Intermediate Compound 39

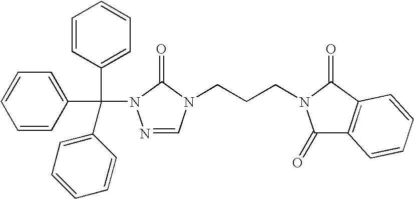

To a mixture of intermediate compound 26 (2.446 mmol) in DMF (2 ml), NaH (4.077 mmol) was added. The reaction was stirred for 30 minutes at room temperature. Then 2-(3-bromopropyl)-1H-isoindole-1,3(2H)-dione (2.70 mmol) was added and the reaction was heated for 20 hours at 90° C. Then the solvent was evaporated and the product was purified by column chromatography (eluent: CH₂Cl₂/CH₃OH 9/1). Selected fractions were collected and their solvent evaporated. Yield: 94 0 mg of intermediate compound 39 (75%).

### c. Preparation of Intermediate Compound 58

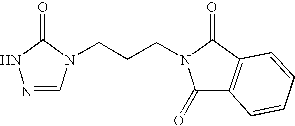

A mixture of intermediate compound 39 (0.972 mmol) in TFA/H₂O/CH₂Cl₂ (1:1:1) (10 ml) was stirred for 20 hours at 60° C. The solvent was removed and the product was purified by column chromatography (eluent: CH₂Cl₂/CH₃OH 9/1). Selected fractions were collected and their solvent evaporated. Yield: 265 mg of intermediate compound 58 (100%). This crude was used in the next step without further purification.

### d. Preparation of Intermediate Compound 59

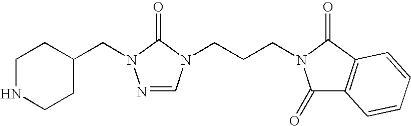

To a mixture of intermediate compound 58 (0.0367 mmol) in CH₂Cl₂ (4 ml), PS-PPh₃ (0.0734 mmol) and 1,1-dimethylethyl 4-(hydroxymethyl)-1-piperidinecarboxylic acid ester (0.5505 mmol) were added. The reaction was stirred for 5 minutes. Then bis(1,1-dimethylethyl)diaznedicarboxylic acid ester (0.05505 mmol) was added and the reaction was stirred for 3 hours at room temperature. The resin was filtered off and the filtrate solvent was evaporated. The product was purified by column chromatography (eluent: CH₂Cl₂/MeOH 9/1). Selected fractions were collected and their solvent evaporated. The residue was treated with CH₂Cl₂/trifluoroacetic acid 8/2 and the solvent evaporated. Yield: 25 mg of intermediate compound 59 (100%). This crude was used in the next step without further purification.

### Example A7

### a. Preparation of Intermediate Compound 29

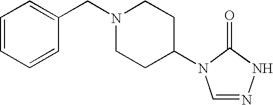

A mixture of 1-(phenylmethyl)-4-piperidineamine (0.0125 mol) and methyl 2-[(dimethylamino)methylene]hydrazinecarboxylate (0.025 mol) in CH₃CN (50 ml) was reacted under microwave conditions for 20 minutes at 180° C. and then the solvent was evaporated. Finally, the obtained residue was washed with CH₃CN/ethyl ether (1/9). Yield: 2.6 g of intermediate compound 29 (81%).

### b. Preparation of Intermediate Compound 30

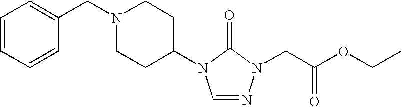

A mixture of intermediate compound 29 (0.003 mol), bromo acetic acid ethyl ester (0.0045 mol) and K₂CO₃ (0.006 mol) in CH₃CN (10 ml) was heated under microwave conditions for 15 minutes at 120° C. and then CH₂Cl₂ was added. The solids were filtered off and the organic solvent was evaporated. The residue was purified by short open column chromatography (eluent 1: CH₂Cl₂/EtOAc 1/1; eluent 2: CH₂Cl₂/CH₃OH 96/4). The product fractions were collected and the solvent was evaporated. Yield: 1.01 g of intermediate compound 30 (98%).

### c. Preparation of Intermediate Compound 31

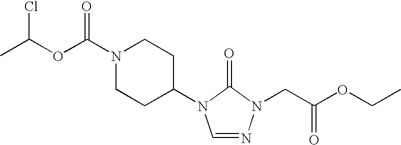

A mixture of intermediate compound 30 (0.0029 mol) and 1-chloroethyl carbonochloride acid ester (0.0087 mol) in THF (10 ml) was stirred and refluxed for 1 hour, then extra 1-chloroethyl carbonochloride acid ester (0.939 ml) was added and the reaction mixture was heated for 1 hour. A saturated NaHCO₃ solution was added and the mixture was extracted with CH₂Cl₂. The organic layer was separated, dried (Na₂SO₄), filtered off and the solvent was evaporated. The residue was purified by short open column chromatography over silica gel (eluent 1: CH₂Cl₂/EtOAc 1/0, 1/1; eluent 2: CH₂Cl₂/CH₃OH 9/1). The product fractions were collected and the solvent was evaporated. Yield: 0.28 g of intermediate compound 31.

### d. Preparation of Intermediate Compound 32

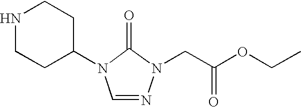

A mixture of intermediate compound 31 (0.00078 mol) in CH₃OH (10 ml) was stirred and refluxed for 1 hour and then CH₂Cl₂ and a saturated Na₂CO₃ solution were added. The organic layer was separated, dried (Na₂SO₄), filtered off and the solvent was evaporated. Yield: 0.200 g of intermediate compound 32.

### e. Preparation of Intermediate Compound 33

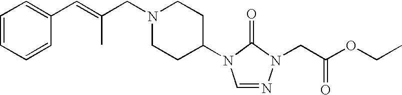

A mixture of intermediate compound 32 (0.00079 mol), 2-methyl-3-phenyl-2-propenal (0.00119 mol) and NaBH(Oac)₃ (0.00119 mol) in dichloroethane (4 ml) was heated for 10 minutes at 105° C. and then NH₄OH (38% NH₃ in H₂O) was added. The organic layer was separated, dried (Na₂SO₄), filtered off and the solvent was evaporated. The residue was purified by short open column chromatography in a manifold (eluent 1: CH₂Cl₂/EtOAc 9/1; eluent 2: CH₂Cl₂/CH₃OH 96/4). The product fractions were collected and the solvent was evaporated. Yield: 0.055 g of intermediate compound 33 (18%).

### f. Preparation of Intermediate Compound 14

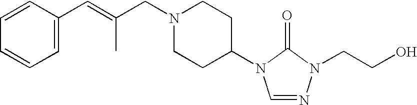

NaBH₄ (0.00035 mol) was added to a mixture of intermediate 33 (0.00014 mol) in CH₃OH (150 ml) and THF (0.750 ml) and then the reaction mixture was stirred for 1 hour at room temperature. A 10% NH₄Cl solution was added and the mixture was extracted with CH₂Cl₂. The organic layer was separated, dried (Na₂SO₄), filtered off and the solvent was evaporated. Yield: 0.038 g of intermediate compound 14 (79%).

### g. Preparation of Intermediate Compound 15

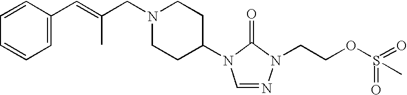

Methylsulfonyl chloride (0.00017 mol) was added at 0° C. to a solution of intermediate compound 14 (prepared according to A7.a) (0.00011 mol) and Et₃N (0.00022 mol) in CH₂Cl₂ (1 ml), then the reaction mixture was stirred for 1 hour at room temperature and a saturated NaHCO₃ solution was added. The organic layer was separated, dried (Na₂SO₄), filtered off and the solvent was evaporated. The residue was percolated in a manifold (5 g cartridge) (eluent: CH₂Cl₂/CH₃OH 97/3). The product fractions were collected and the solvent was evaporated. Yield: 0.033 g of intermediate compound 15 (71%).

### Example A8

### a. Preparation of Intermediate Compound 16

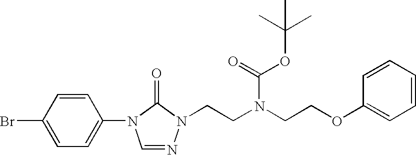

Bis(1,1-dimethylethyl) dicarbonic acid ester (0.0094 mol) was added at 0° C. to a solution of intermediate

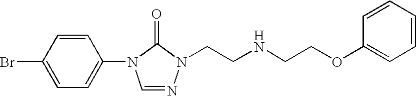

(prepared according to B8.a) (0.0094 mol) and CH₂Cl₂ (0.0094 mol) in Et₃N (25 ml) and the reaction mixture was stirred for 1 hour at 0° C. and then at room temperature. The solvent was evaporated and the residue was purified by short open column chromatography over silica gel (eluent gradient: CH₂Cl₂/EtOAc 1/0, 1/1). The product fractions were collected and the solvent was evaporated. Yield: 3.5 g of intermediate compound 16 (74%).

### b. Preparation of Intermediate Compound 17

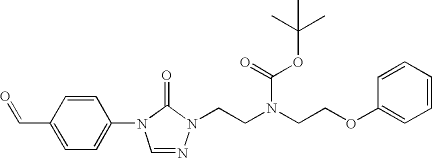

Intermediate compound 16 (prepared according to A8.a) (5.5 mmol), NaHCO₂ (16.5 mmol), PdCl₂(PPh₃)₂ (0.28 mmol) and DMF (50 mL) were introduced in a PARR vessel. The system was closed and pressurized with CO (gas) (40 bars). The reaction was heated at 120° C. for 20 hours. Then the system was opened. The solvent was evaporated and solid was taken up into CH₂Cl₂ filtered off through CELITE. The filtrate solvent was evaporated and the residue was purified by column chromatography (eluent gradient: CH₂Cl₂/AcOEt 9/1 and 4/1). Selected fractions were collected and their solvent evaporated. Yielding 1.1 g of intermediate compound 17 (27%).

### c. Preparation of Intermediate Compound 18

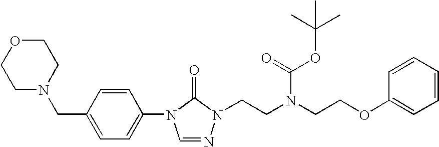

A mixture of intermediate compound 17 (prepared according to A8.b) (0.00029 mol), morpholine (0.00044 mol) and NaBH(OAc)₃ (0.00044 mol) in dichloroethane (2 ml) was reacted for 10 minutes at 100° C. and then a concentrated NH₄OH solution was added. The organic layer was separated, dried (Na₂SO₄), filtered off and the solvent was evaporated. The residue was percolated in a silica cartridge (5 g) (eluent 1: CH₂Cl₂/EtOAc 4/1; eluent 2: CH₂Cl₂/CH₃OH 96/4). The product fractions were collected and the solvent was evaporated. Yield: 0.122 g of intermediate compound 18 (80%).

### Example A9

### a. Preparation of Intermediate Compound 19

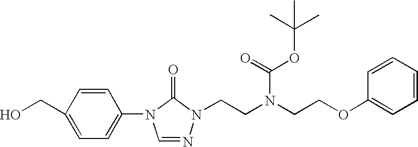

NaBH₄ (0.00031 mol) was added to a solution of intermediate compound 17 (prepared according to A8.b) (0.00031 mol) in CH₃OH (2 ml) at 0° C. and the reaction mixture was stirred for 1 hour at room temperature. A 10% NH₄Cl solution was added and the mixture was extracted with CH₂Cl₂. The organic layer was separated, dried (Na₂SO₄), filtered off and the solvent was evaporated. The residue was purified in a manifold (vacuum) using a Sep-Pak silica cartridge (5 g) (eluent 1: CH₂Cl₂/EtOAc 4/1; eluent 2: CH₂Cl₂/2-propanone 2/1). The product fractions were collected and the solvent was evaporated. Yield: 0.054 g of intermediate compound 19 (38%).

### b. Preparation of Intermediate Compound 20

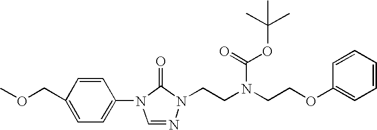

Reaction under N₂: a mixture of intermediate compound 19 (prepared according to A9.a) (0.00012 mol) in dry THF (1 ml) was added at 0° C. to a mixture of 60% NaH (0.00024 mol) in dry THF (1 ml) and the resulting mixture was stirred for 15 minutes, then CH₃I (0.00048 mol) was added at 0° C. and the reaction mixture was stirred for 90 minutes at room temperature. Extra CH₃I (0.00048 mol) was added and the mixture was stirred for 90 minutes at room temperature, then a 10% NH₄Cl solution was added and the resulting mixture was extracted with CH₂Cl₂. The organic layer was separated, dried (Na₂SO₄), filtered off and the solvent was evaporated. The residue was purified in a manifold (vacuum) using a Sep-Pak silica cartridge (5 g) (eluent gradient: CH₂Cl₂/EtOAc 1/0, 4/1). The product fractions were collected and the solvent was evaporated. Yield: 0.047 g of intermediate compound 20 (84%).

### Example A10

### a. Preparation of Intermediate Compound 21

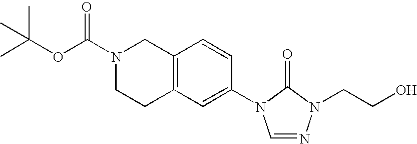

NaBH₄ (0.0170 mol) was added portionwise at 0° C. to a solution of intermediate compound

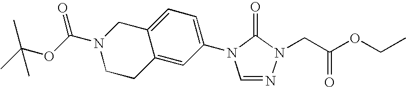

(prepared according to A7.e) (0.0068 mol) in CH₃OH (7 ml) and dry THF (35 ml) and then the reaction mixture was stirred for 1 hour at room temperature. A 10% NH₄Cl solution was added and the mixture was extracted with CH₂Cl₂. The organic layer was separated, dried (Na₂SO₄), filtered off and the solvent was evaporated. Yield: 2.13 g of intermediate compound 21 (87%).

### b. Preparation of Intermediate Compound 22

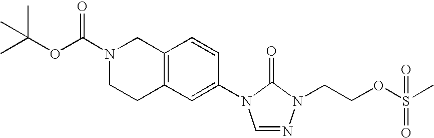

Methylsulfonyl chloride (0.0087 mol) was added dropwise at 0° C. to a mixture of intermediate compound 21 (prepared according to A10.a) (0.0058 mol) and Et₃N (0.0116 mol) in CH₂Cl₂ (35 ml) and then the reaction mixture was stirred for 1 hour at room temperature. A saturated NaHCO₃ solution was added and the mixture was extracted with CH₂Cl₂. The organic layer was separated, dried (Na₂SO₄), filtered off and the solvent was evaporated. The residue was precipitated from DIPE and then the resulting solids were collected. Yield: 2.5 g of intermediate compound 22 (98%).

### c. Preparation of Intermediate Compound 23

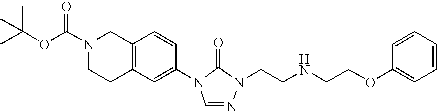

A mixture of intermediate compound 22 (prepared according to A10.b) (0.0055 mol), 2-phenoxyethanamine (0.0110 mol), Cs₂CO₃ (0.0110 mol) and Molecular Sieves 4 (0.5 g) in CH₃CN (40 ml) was heated for 20 minutes at 150° C. under microwave irradiation, then CH₂Cl₂ was added and the reaction mixture was filtered over celite. The solvent was evaporated and the obtained residue was purified by short open column chromatography (eluent: CH₂Cl₂/(CH₃OH/NH₃) 97/3). The product fractions were collected and the solvent was evaporated. Yield: 2.330 g of intermediate compound 23 (88%).

### d. Preparation of Intermediate Compound 24

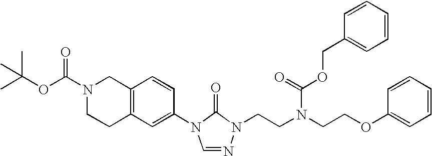

A mixture of intermediate compound 23 (prepared according to A10.c) (0.0045 mol) and NaH (60%) (0.0068 mol) in THF (25 ml) was stirred for 3 hours at room temperature and under N₂, then benzyl chloroformate (0.0068 mol) was added and the reaction mixture was stirred for 3 hours at room temperature. EtOAc was added and the organic layer was washed with water and with brine, then dried (Na₂SO₄), filtered and the solvent was evaporated. The obtained residue was purified by short open column chromatography (eluent gradient: CH₂Cl₂/EtOAc 4/1, 2/1). The product fractions were collected and the solvent was evaporated. Yield: 2.14 g of intermediate compound 24 (78%).

### e. Preparation of Intermediate Compound 25

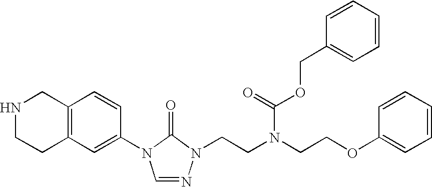

Trifluoroacetic acid (20 ml) was added dropwise to a mixture of intermediate compound 24 (prepared according to A10.d) (0.0034 mol) in CH₂Cl₂ (240 ml), then the reaction mixture was stirred for 1 hour at room temperature and the solvent was evaporated. The obtained residue was alkalised with a satd. Na₂CO₃ solution and the resulting mixture was extracted with CH₂Cl₂. The organic layer was separated, dried (Na₂SO₄), filtered off and the solvent was evaporated. Yield: 1.78 g of intermediate compound 25.

### f. Preparation of Intermediate Compound 34

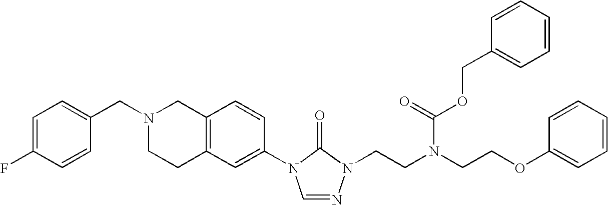

A mixture of intermediate compound 25 (0.0002921 mol), 1-(chloromethyl)-4-fluorobenzene (0.0008763 mol) and polymer supported TBD (2.9 mmol/g) (0.0008763 mol) in CH₃CN (2 ml) and DMF (0.15 ml) was reacted under microwave conditions for 20 min. at 170° C. and then the reaction mixture was filtered and the filter residue was washed with CH₂Cl₂. The solvent was evaporated and the obtained residue was purified in a manifold (vacuum) using a Sep-Pak silica cartridge (eluent: CH₂Cl₂/(CH₃OH/NH₃) 99/1). The product fractions were collected and the solvent was evaporated Yield: 0.140 g of intermediate compound 34 (77%).

### Example A11

### Preparation of Intermediate Compound 35

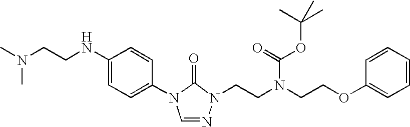

A mixture of intermediate compound 8 (prepared according to A8.a) (0.0002 mol), N,N-dimethyl-1,2-ethanediamine (0.0003 mol), Pd(Oac)₂ (0.00001 mol), Xantphos (0.00002 mol) and Cs₂CO₃ (0.0003 mol) in deoxygenated dioxane (1 ml) was heated for 15 minutes at 150° C. and then for 10 minutes at 170° C. The solids were filtered off and the solvent was evaporated. The residue was purified in a manifold (eluent 1: CH₂Cl₂/CH₃OH 96/4; eluent 2: CH₂Cl₂/(CH₃OH/NH₃) 95/5). The product fractions were collected and the solvent was evaporated. Yield: 0.040 g of intermediate compound 35 (39%).

### Example A12

### a. Preparation of Intermediate Compound 36

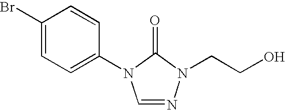

NaBH₄ 50.0085 mol) was added at 0° C. to a mixture of 4-(4-bromophenyl)-4,5-dihydro-5-oxo-1H-1,2,4-triazole-1-acetic acid ethyl ester (0.0034 mol) in CH₃OH (3 ml) and THF (15 ml) and then the reaction mixture was stirred for 2 hours at room temperature. A 10% NH₄Cl solution was added and the resulting mixture was extracted with CH₂Cl₂. The organic layer was separated, dried (Na₂SO₄), filtered off and the solvent was evaporated. The residue was washed with DIPE and the desired product was collected. Yield: 0.78 g of intermediate compound 36 (81%).

### b. Preparation of Intermediate Compound 37

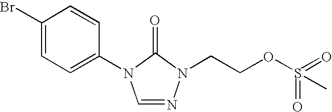

Methanesulfonyl chloride (0.015 mol) was added portionwise at 0° C. to a mixture of intermediate compound 36 (prepared according to A12.a) (0.01 mol) and Et₃N (0.02 mol) in CH₂Cl₂ (50 ml), then the reaction mixture was stirred for 1 hour at room temperature and a saturated NaHCO₃ solution was added. The organic layer was separated, dried (Na₂SO₄), filtered off and the solvent was evaporated. Finally, the residue was washed with ethyl ether. Yield: 3.39 g of intermediate compound 37 (94

### c. Preparation of Intermediate Compound 38

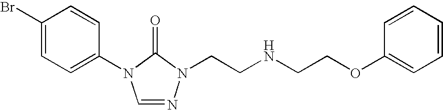

A mixture of intermediate compound 37 (prepared according to A12.b) (0.0094 mol), 2-phenoxyethanamine (0.0188 mol), N,N,N-tributyl-1-butanaminium bromide (0.0094 mol) and K₂CO₃ (0.0188 mol) in CH₃CN (40 ml) was heated for 20 minutes at 120° C. and then CH₂Cl₂ was added. The solids were filtered off and the filter residue was purified by column chromatography (eluent 1: CH₂Cl₂/EtOAc 1/1; eluent 2: CH₂Cl₂/CH₃OH 96/4). The product fractions were collected and the solvent was evaporated. Yield: 5.9 g of intermediate compound 38 (Quantitative Yield, used as such in the next reaction step without further purification).

### d. Preparation of Intermediate Compound 40

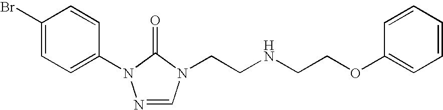

A mixture of intermediate compound 39

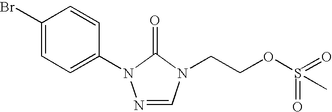

(0.001518 mol), 2-phenoxyethanamine (0.0030 mol) and Cs₂CO₃ (0.0030 mol) in dry CH₃CN (10 ml) was stirred in a microwave oven (Milestone) for 20 minutes at 150° C., then the cooled reaction mixture was filtered over celite and the filtrate was evaporated. The residue was purified by open column chromatography over silica gel (eluent 1: CH₂Cl₂/EtOAc 1/1; eluent 2: CH₂Cl₂/CH₃OH 96/4). The product fractions were collected and the solvent was evaporated. Yield: 0.52 g of intermediate compound 40 (85%).

### Example A13

### Preparation of Intermediate Compound 41

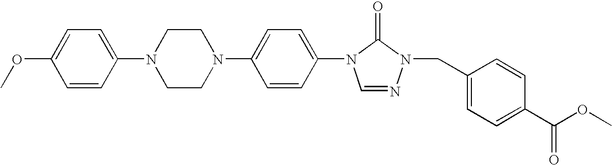

A mixture of intermediate compound 11 (prepared according to A5.a) (0.0014 mol), 4-(bromomethyl)-benzoic acid methyl ester (0.0021 mol) and K₂CO₃ (0.028 mol) in CH₃CN (10 ml) was heated in a microwave oven for 15 minutes at 150° C., then CH₂Cl₂ was added and the reaction mixture was filtered over dicalite. The filtrate's solvent was evaporated and the resulting residue was washed with EtOAc. Yield: 0.335 g of intermediate compound 41 (49%).

### Example A14

### a. Preparation of Intermediate Compound 43

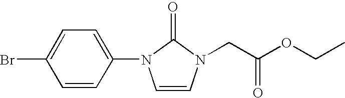

Reaction in microwave oven. A mixture of 1-(4-bromophenyl)-1,3-dihydro-2H-imidazol-2-one (prepared according to the teachings in WO2003042188 of which the content is included herein) (0.0058 mol), 2-bromoacetic acid ethyl ester (0.0070 mol) and K₂CO₃ (0.0087 mol) in CH₃CN (20 ml) was heated for 15 minutes at 130° C. CH₂Cl₂ was added. The precipitate was filtered off through Celite and the filtrate's solvent was evaporated. Yield: 2.0 g of intermediate compound 43 (quantitative yield; used in next reaction step, without further purification).

### b. Preparation of Intermediate Compound 44

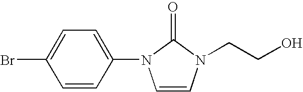

NaBH₄ (0.0145 mol) was added portionwise to a solution of intermediate compound 43 (0.0058 mol) in CH₃OH (4 ml) and THF (20 ml), stirred at 0° C. The reaction mixture was stirred for one hour at room temperature. A 10% aqueous NH₄Cl solution was added. This mixture was extracted with CH₂Cl₂. The separated organic layer was dried (Na₂SO₄), filtered and the solvent evaporated. Yield: 1.28 g of intermediate compound 44 (78%).

### c. Preparation of Intermediate Compound 45

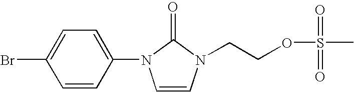

Methane sulfonylchloride (0.0050 mol) was added portionwise to a solution of intermediate compound 44 (0.0045 mol) and Et₃N (0.0068 mol) in CH₂Cl₂ (15 ml), stirred at 0° C. The resultant reaction mixture was stirred for one hour at room temperature. A saturated aqueous NaHCO₃ solution was added. The organic layer was separated, dried (Na₂SO₄), filtered and the solvent was evaporated. The residue was treated with diethyl ether. The precipitate was filtered off and dried. Yield: 1.43 g of intermediate compound 45 (88%).

### d. Preparation of Intermediate Compound 46

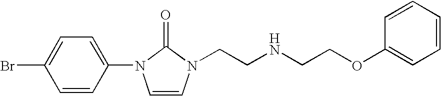

Reaction in microwave oven. A mixture of intermediate compound 45 (0.0037 mol), 2-phenoxyethanamine (0.0074 mol), Cs₂CO₃ (0.0074 mol) and 4 Å molecular sieves (0.330 g) in CH₃CN (25 ml) was heated for 20 minutes at 170° C. The precipitate was filtered off through dicalite and the filtrate's solvent was evaporated. The residue was purified by short open column chromatography over silica gel (eluent: CH₂Cl₂/CH₃OH 94/6). The product fractions were collected and the solvent was evaporated. Yield: 1.4 g of intermediate compound 46 (94%).

### e. Preparation of Intermediate Compound 47

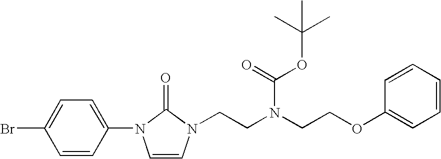

Bis(1,1-dimethylethyl)-dicarbonic acid ester (0.0035 mol) was added at a mixture of intermediate compound 46 (0.0035 mol) and Et₃N (0.0035 mol) in CH₂Cl₂ (15 ml), stirred at 0° C. Then, the reaction mixture was stirred for one hour at room temperature. The product was purified by short open column chromatography over silica gel (eluent: CH₂Cl₂/EtOAc 100/0, then 3/1). The product fractions were collected and the solvent was evaporated. The residue was treated with DIPE, and then dried. Yield: 1.23 g of intermediate compound 47 (70%).

### f. Preparation of Intermediate Compound 48

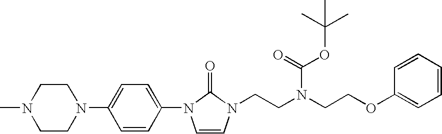

Reaction in microwave oven. Reaction under N₂ atmosphere. A mixture of intermediate compound 47 (0.00033 mol), 1-methylpiperazine (0.0005 mol), Pd(Oac)₂ (0.000017 mol), Xantphos (0.000033 mol) and Cs₂CO₃ (0.0005 mol) in dioxane/DMF 9/1, deoxygenated (1.5 ml) was heated for 15 minutes at 175° C. CH₂Cl₂ was added. The solid was filtered off though Celite. The filtrate's solvent was evaporated. The residue was purified by column chromatography over a 10-g silica gel cartridge (eluent: CH₂Cl₂/CH₃OH 97/3; then eluent: CH₂Cl₂/(CH₃OH/NH₃) 96/4 and 95/5). The product fractions were collected and the solvent was evaporated. Yield: 0.085 g of intermediate compound 48 (49%).

### Example A15

### a. Preparation of Intermediate Compound 49

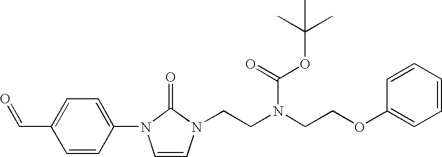

Reaction under N₂ atmosphere. N-(1,1-dimethylethyl)-N-ethyl-2-methyl-2-propanamine (0.0024 mol) was added to a solution of intermediate compound 47 (prepared according to A14.e) (0.0012 mol), acetyl formy anhydride (0.0024 mol), PdCl₂(PPh₃)₂ (0.00012 mol) and triethylsilane (0.0018 mol) in CH₃CN, dry (12 ml). In a sealed tube, the reaction mixture was heated for 24 hours at 60° C. Extra acetyl formy anhydride, PdCl₂(PPh₃)₂, triethylsilane and N-(1,1-dimethylethyl)-N-ethyl-2-methyl-2-propanamine was added. The reaction mixture was heated for 24 hours at 60° C. The precipitate was filtered off through Celite, then rinsed with CH₂Cl₂ and the filtrate's solvent was evaporated. The residue was purified by short open column chromatography over silica gel (eluent: CH₂Cl₂/EtOAc 100/0, then 4/1). The product fractions were collected and the solvent was evaporated. Yield: 0.400 g of intermediate compound 49 (74%).

### b. Preparation of Intermediate Compound 50

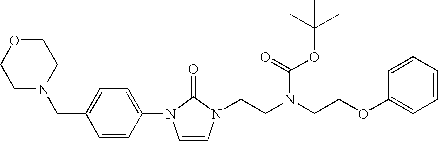

Reaction in microwave oven. A mixture of intermediate compound 49 (0.00044 mol), morpholine (0.00075 mol) and NaBH(OAc)₃ (0.00075 mol) in 1,2-dichloroethane (2 ml) was heated for 15 minutes at 80° C. A 32% aqueous NH₃ solution was added. The organic layer was separated, dried (Na₂SO₄), filtered and the solvent was evaporated. The residue was purified by column chromatography in a Manifold (eluent: CH₂Cl₂/EtOAc 1/1, then CH₂Cl₂/CH₃OH 95/5). The product fractions were collected and the solvent was evaporated. Yield: 0.160 g of intermediate compound 50 (70%).

### Example A16

### a. Preparation of Intermediate Compound 51

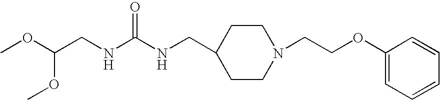

A solution of trichloromethanol carbonate (0.008 mol) in CH₂Cl₂, dry (25 ml) was added dropwise to a solution of 2,2-dimethoxymethanamine (0.022 mol) in CH₂Cl₂, dry (50 ml), stirred at 0° C. Et₃N (0.044 mol) was added in three portions, while stirring at 0° C. The reaction mixture was stirred for 5 minutes at room temperature. A solution of 1-(phenoxyethyl)-4-piperidinemethanamine (0.011 mol) in CH₂Cl₂, dry (25 ml) was added and the resultant reaction mixture was stirred for one hour at room temperature. A saturated aqueous Na₂CO₃ solution was added. The organic layer was separated, dried (Na₂CO₃), filtered and the solvent was evaporated. Yield: 5.6 g of intermediate compound 51 (quantitative yield; used in next reaction step, without further purification).

### b. Preparation of Intermediate Compound 52

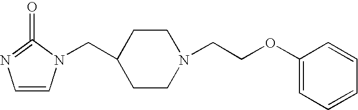

Reaction in microwave oven. A mixture of intermediate compound 51 (0.011 mol) in HCl, 2N (20 ml) and CH₃OH (50 ml) was heated for 10 minutes at 120° C. The mixture was poured out into a saturated aqueous Na₂CO₃ solution. This mixture was extracted with CH₂Cl₂. The separated organic layer was dried (Na₂SO₄), filtered and the solvent was evaporated. The residue was purified by short open column chromatography over silica gel (eluent: CH₂Cl₂/(CH₃OH/NH₃) 96/4, then 90/10). The product fractions were collected and the solvent was evaporated. The residue was treated with diethyl ether, then collected and dried. Yield: 1.35 g of intermediate compound 52 (41%).

### c. Preparation of Intermediate Compound 53

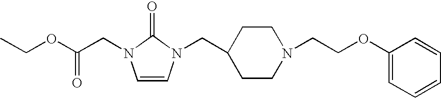

Reaction in microwave oven. A mixture of intermediate compound 52 (0.0010 mol), chloro acetic acid ethyl ester (0.0015 mol) and K₂CO₃ (0.0015 mol) in CH₃CN (4 ml) was heated for 15 minutes at 120° C., then for 15 minutes at 150° C. The precipitate was filtered off, then purified by column chromatography in a Manifold (eluent: CH₂Cl₂/CH₃OH 95/5). The product fractions were collected and the solvent was evaporated. Yield: 0.243 g of intermediate compound 53 (63%).

### d. Preparation of Intermediate Compound 54

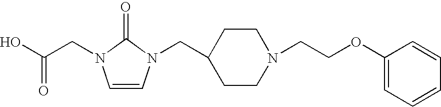

A solution of LiOH (0.00076 mol) in H₂O (1 ml) was added to a solution of intermediate compound 53 (0.00063 mol) in dioxane (10 ml). The resultant reaction mixture was stirred for 24 hours at room temperature. The solvent was evaporated. The residue was treated with diethyl ether, then collected and dried. Yield: 0.190 g of intermediate compound 54 (83%).

### Example A 17

### a. Preparation of Intermediate Compound 42

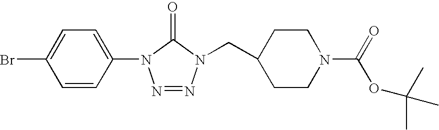

Reaction in microwave oven. A mixture of 1-(4-bromophenyl)-1,2-dihydro-5H-tetrazol-5-one (0.0058 mol), 4-(iodomethyl)-1-piperidinecarboxylic acid 1,1-dimethylethyl ester (0.0070 mol) and BTTP (0.0070 mol) in CH₃CN (20 ml) was heated for 30 minutes at 120° C. The solvent was evaporated. The residue was purified by short open column chromatography over silica gel (eluent: CH₂Cl₂/EtOAc 100/0, then 4/1). The product fractions were collected and the solvent was evaporated. Yield: 2.58 g of intermediate compound 42.

### b. Preparation of Intermediate Compound 55

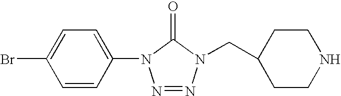

Trifluoroacetic acid (10 ml) was added to a solution of intermediate compound 42 (prepared according to A17) (0.0058 mol) in CH₂Cl₂ (40 ml).The reaction mixture was stirred for 2 hours at room temperature. A saturated aqueous Na₂CO₃ solution was added (pH=8). The organic layer was separated, dried (Na₂SO₄), filtered and the solvent was evaporated. The residue was treated with diethyl ether, then collected and dried. Yield: 0.85 g of intermediate compound 55 (45%).

### c. Preparation of Intermediate Compound 56

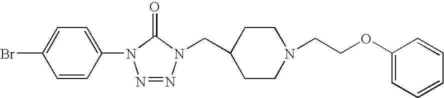

Reaction in microwave oven. A mixture of intermediate compound 55 (prepared according to A17.b) (0.0025 mol), 2-bromoethoxybenzene (0.00275 mol) and K₂CO₃ (0.0050 mol) in CH₃CN (10 ml) was heated for 10 minutes at 120° C. CH₂Cl₂ was added. The precipitate was filtered off through Celite and the filtrate's solvent was evaporated. The residue was treated with diethyl ether, then collected and dried. Yield: 0.85 g of intermediate compound 56 (74%).

### d. Preparation of Intermediate Compound 57

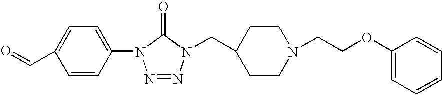

Reaction under N₂ atmosphere. N-ethyl-N-(1-methylethyl)-2-propanamine (0.0030 mol) was added to a mixture of intermediate compound 56 (prepared according to A17.d) (0.0015 mol), acetyl formate (0.0030 mol), dichlorobis(triphenylphosphine) palladium (0.00015 mol) and triethylsilane (0.00225 mol) in CH₃CN, dry (15 ml). In a sealed tube, the reaction mixture was heated for 24 hours at 60° C. Extra acetyl formate, dichlorobis(triphenylphosphine) palladium, triethylsilane and N-ethyl-N-(1-ethylethyl)-2-propanamine was added. The reaction mixture was heated for 24 hours at 60° C. The precipitate was filtered off and the filtrate's solvent was evaporated. The residue was purified by short open column chromatography over silica gel (eluent: CH₂Cl₂/EtOAc 4/1, then CH₂Cl₂/CH₃OH 9/1). The product fractions were collected and the solvent was evaporated. Yield: 0.660 g of intermediate compound 57.

This intermediate compound 57 is used as starting material to prepare final compound 104.

The following intermediate structures in Table 1 were made according to examples described above:

TABLE 1

| Exp. nr. | Structure |
|---|---|
| A1.a-2 | 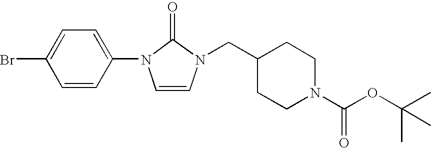 |
| A1.b | 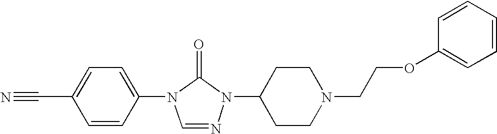 |
| A1.c | 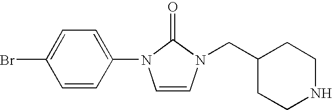 |
| A1.c | 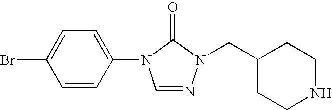 |
| A1.e | 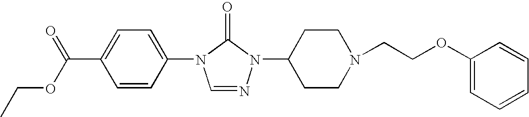 |
| A1.e | 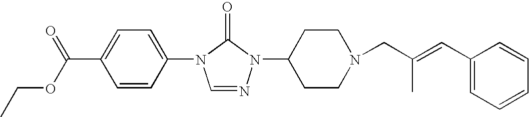 |
| A1.f | 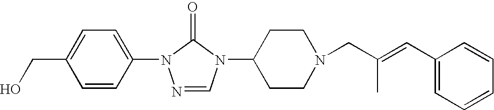 |
| A1.f | 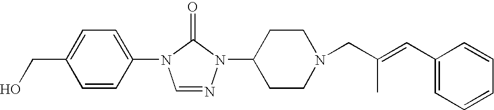 |
| A1.f | 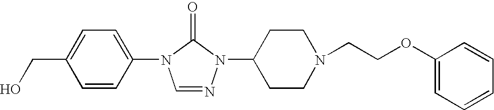 |
| A1.f | 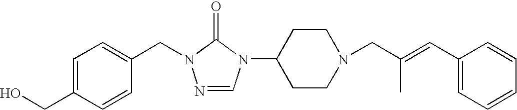 |
| A1.f | 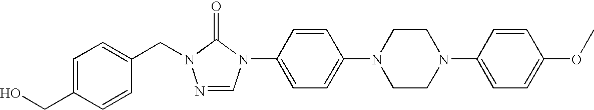 |
| A1.f | 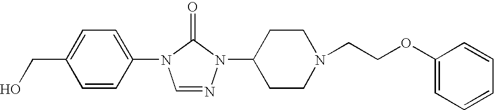 |
| A2.c | 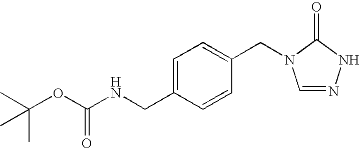 |
| A3 | 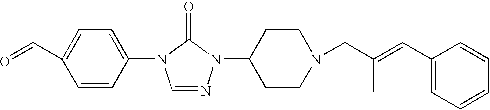 |
| A3 | 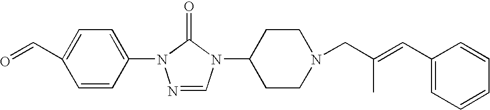 |
| A4.a | 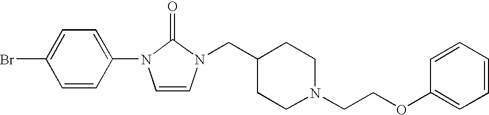 |
| A4.a | 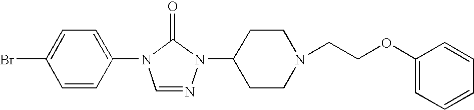 |
| A4.a | 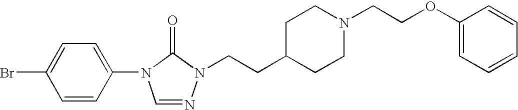 |
| A5.b | 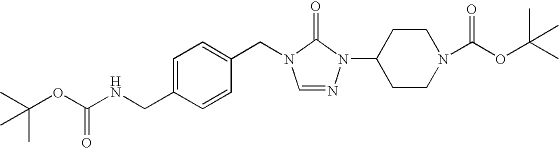 |
| A.6.d | 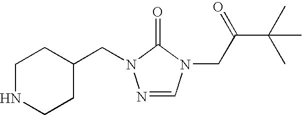 |
| A7.e | 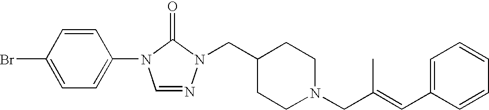 |
| A7.e | 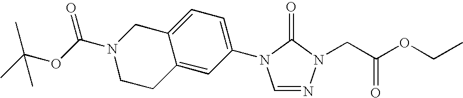 |
| A7.e | 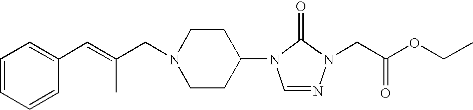 |
| A7.f | 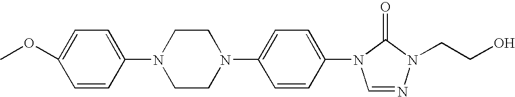 |
| A7.f | 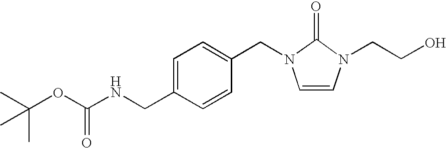 |
| A7.g | 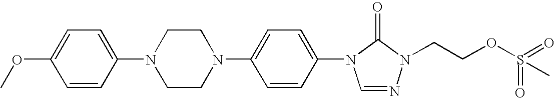 |
| A7.g | 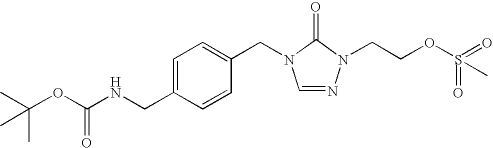 |
| A8.a | 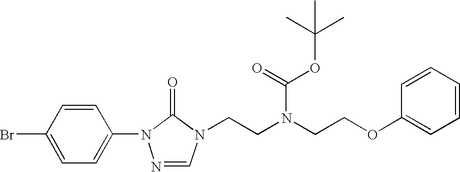 |
| A8.b | 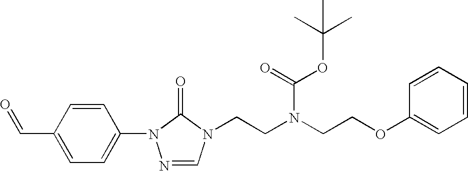 |
| A8.c | 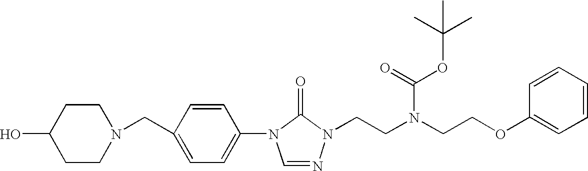 |
| A8.c | 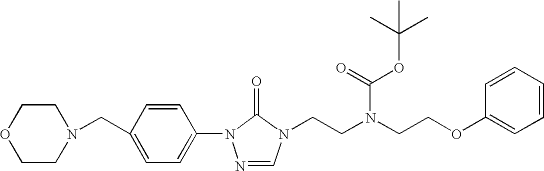 |
| A7.g | 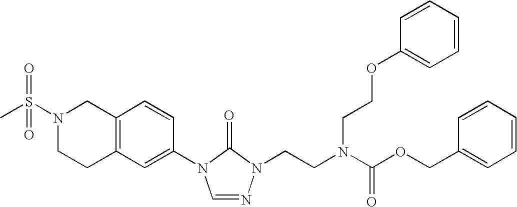 |
| A10.b | 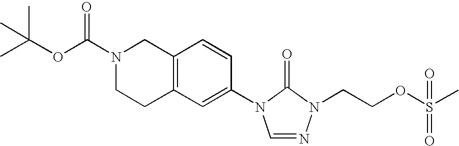 |
| A10.f | 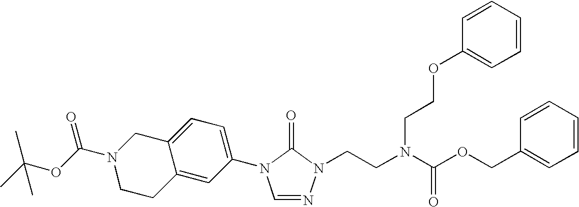 |
| A10.f | 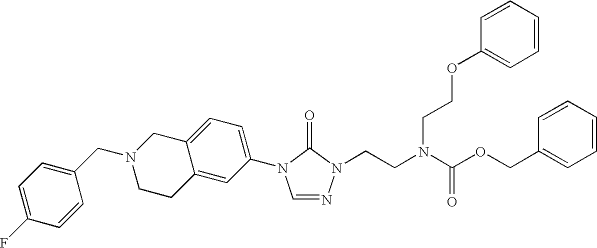 |
| A10.f | 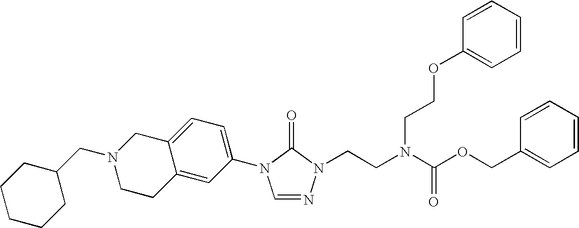 |
| A10.f | 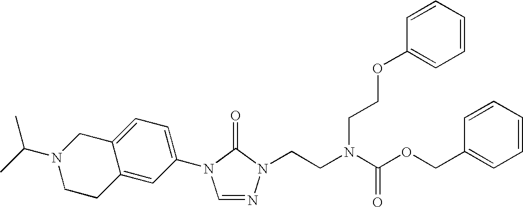 |
| A11 | 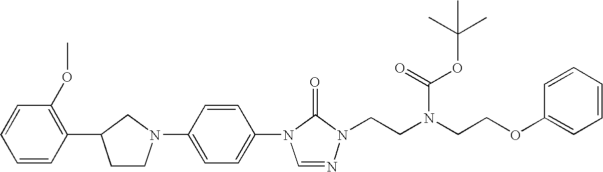 |
| A12.c | 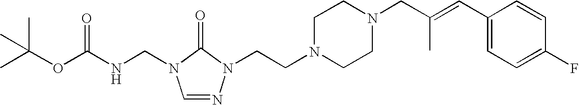 |
| A12.c | 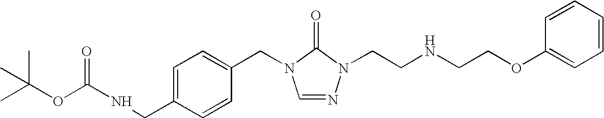 |
| A13 | 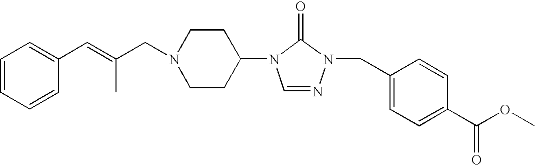 |
| A14.a | 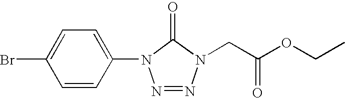 |
| A14.b | 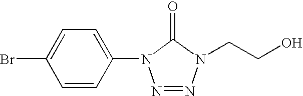 |
| A14.c | 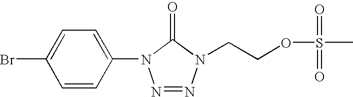 |
| A14.d | 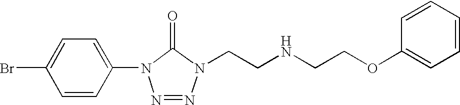 |
| A14.e | 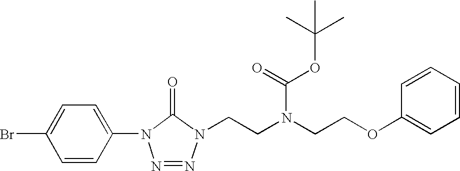 |
| A14.f | 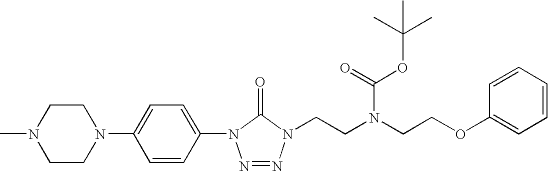 |
| A15.a | 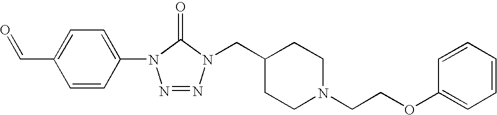 |
| A15.a | 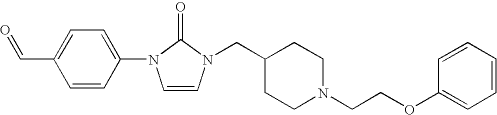 |
| A15.b | 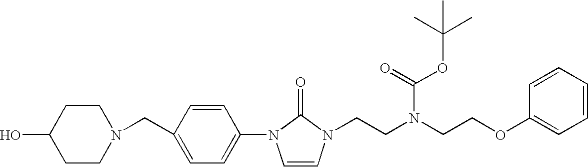 |
| A15.b | 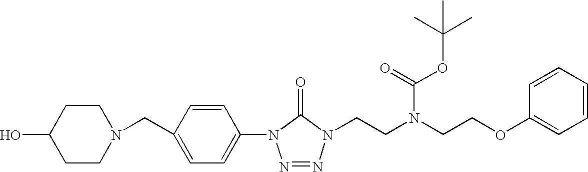 |
| A15.b | 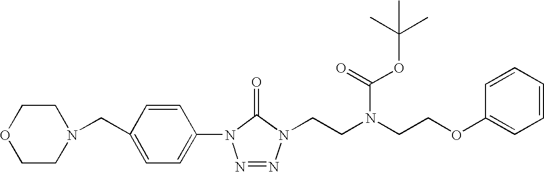 |

The intermediates shown above may be converted into the final compounds according to the invention according to the general reaction scheme

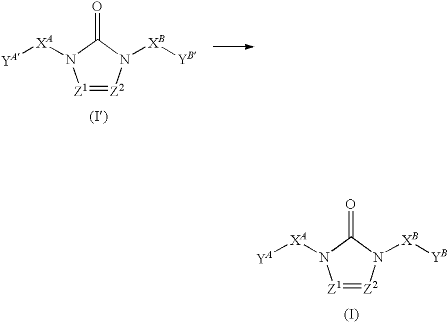

wherein at least one of Y^{A′} and Y^{B′} is selected from the group of halo, in particular Br; formyl; alkylSO₃—; cyano; hydroxy; and alkyloxy, in particular methoxy and ethyloxy; or wherein at least one of Y^{A′} and Y^{B′} is NR¹L^{B}, NL^{A}R² or NL^{A}L^{B}, characterized in that L^{A} and L^{B} are each independently of each other selected from the group of alkyloxycarbonyl, in particular t-butyloxycarbonyl (t-BOC); and arylalkyl-oxycarbonyl, in particular benzyloxycarbonyl. Procedures to convert compounds of Formula (I′) are known to the skilled person. A number of procedures will be exemplified herein below. The specific details of these procedures are not limiting to their general applicability.

The invention also relates to an intermediate compound according to Formula (I′)

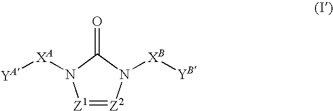

wherein in that at least one of Y^{A′} and Y^{B′} is selected from the group of halo, in particular Br; formyl; alkylSO₃—; cyano; hydroxy; and alkyloxy, in particular methoxy and ethyloxy; or wherein at least one of Y^{A′} and Y^{B′} is NR¹L^{B}, NL^{A}R² or NL^{A}L^{B}, characterized in that L^{A} and L^{B} are each independently of each other selected from the group of alkyloxycarbonyl, in particular t-butyloxycarbonyl (t-BOC); and arylalkyloxycarbonyl, in particular benzyloxycarbonyl.

### B. Preparation of the Final Compounds

### Example B1

### a. Preparation of Final Compound 109

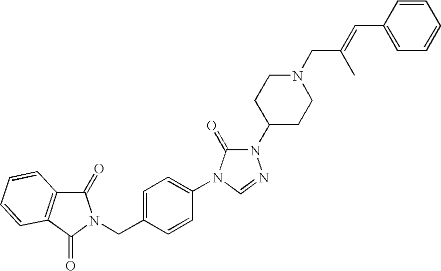

A mixture of intermediate compound 6 (0.000087 mol), phthalimide (0.000261 mol), diethyl azodicarboxylate (0.000261 mol) and PS-triphenylphosphine (0.000348 mol; 3 mmol/g) in dry THF (1 ml) was reacted for 30 minutes at 90° C. and then the resulting solids were filtered off. The filter residue was caught in an ISOLUTE SCX-2 cartridge and released with CH₃OH/NH₃. Yield: 0.018 g of final compound 109 (39%).

### b. Preparation of Final Compound 34

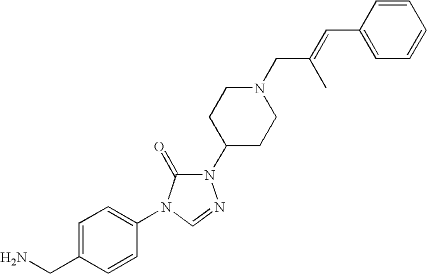

A mixture of final compound 109 (0.000034 mol) and hydrazine (0.000068 mol) in ethanol (1 ml) was stirred and refluxed for 24 hours, then a saturated Na₂CO₃ solution and CH₂Cl₂ were added. The organic layer was separated, dried (Na₂SO₄), filtered off and the solvent was evaporated. The residue was purified using a manifold (eluent 1: CH₂Cl₂/CH₃OH 96/4; eluent 2: CH₂Cl₂/(CH₃OH/NH₃) 96/4). The product fractions were collected and the solvent was evaporated. Yield: 0.006 g of final compound 34 (44%).

### Example B2

### a. Preparation of Final Compound 56

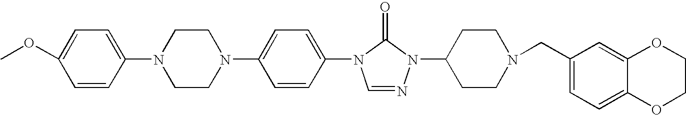

A mixture of final compound 114 (0.00006 mol), 2,3-dihydro-1,4-benzodioxin-6-carboxaldehyde (0.00012 mol) and NaH(OAc)₃ (0.00012 mol) in dichloroethane (1 ml) was heated for 10 minutes at 100° C., then CH₂Cl₂/CH₃OH (1/1) was added and the desired product was caught from the mixture with an ISOLUTE SCX-2 cartridge. The product was washed with CH₂Cl₂/CH₃OH (1/1) and released from the resin with CH₂Cl₂/(CH₃OH/NH₃) (1/1). The solvent was evaporated and the residue was washed with CH₃OH and then collected. Yield: 0.015 g of final compound 56 (43%).

### b. Preparation of Final Compound 80

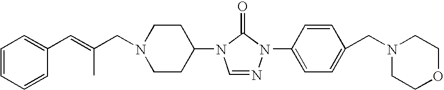

A mixture of intermediate compound 9 (prepared according to A3) (0.00009 mol), morpholine (0.00018 mol) and NaBH(OAc)₃ (0.00014 mol) in dichloroethane (1 ml) was heated in a microwave oven for 10 minutes at 100° C. and then a 31% aqueous NH₃ solution was added. The organic layer was separated, dried (Na₂SO₄), filtered off and the solvent was evaporated. The residue was purified in a manifold (vacuum) (eluent 1: CH₂Cl₂/EtOAc 1/1; eluent 2: CH₂Cl₂/CH₃OH 96/4). The product fractions were collected and the solvent was evaporated and then the obtained residue was lyophilised. Yield: 0.0077 g of final compound 80 (18%).

### Example B3

### Preparation of Final Compound 7

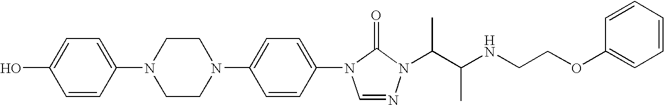

A mixture of intermediate compound

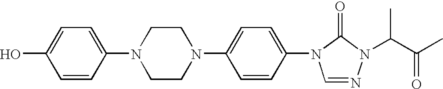

(prepared according to teachings in WO99/58530 of which the content is included herein) (0.025 mol) and 2-phenoxyethanamine (0.036 mol) in THF (300 ml) was hydrogenated at 140° C. for 16 hours with Pd/C 10% (3 g) as a catalyst in the presence of thiophene solution (3 ml). After uptake of H₂ (1 equivalent), the catalyst was filtered off and the filtrate was evaporated. The residue was triturated in 2-propanol. The precipitate was filtered off and dried. Yielding: 12.4 g of final compound 7 (94%).

### Example B4

### a. Preparation of Final Compound 37

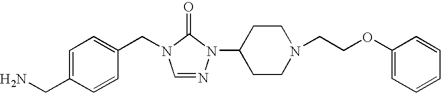

A mixture of compound

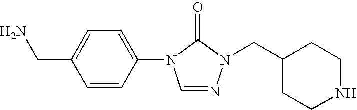

(0.00017 mol), (2-bromo-ethoxy)benzene (0.00011 mol) and CH₃CN (0.00034 mol) in K₂CO₃ (0.5 ml) was heated in a microwave oven for 10 minutes at 120° C., then water was added and the reaction mixture was extracted with CH₂Cl₂. The organic layer was separated, dried (Na₂SO₄), filtered off and the solvent was evaporated. The residue was purified by high-performance liquid chromatography. The product fractions were collected and the solvent was evaporated. Yield: 0.0015 g of final compound 37 (2%).

### b. Preparation of Final Compound 58

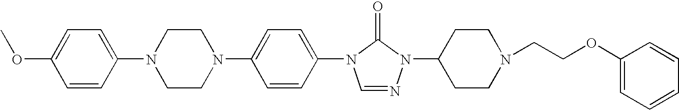

A mixture of intermediate compound

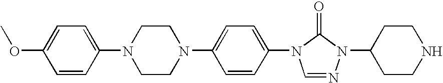

(prepared according to A1.c) (0.00006 mol), (2-bromoethoxy)benzene (0.000072 mol) and K₂CO₃ (0.00012 mol) in CH₃CN (0.5 ml) was heated for 10 minutes under microwave conditions at 120° C., then Resin-linked-N═C═O and CH₂Cl₂ were added and the reaction mixture was stirred for 1 hour at room temperature. The resulting solids were filtered off over dicalite and the desired product was caught from the solution with an ISOLUTE SCX-2 cartridge. The product was washed with CH₂Cl₂/CH₃OH (1/1) and released from the resin with CH₂Cl₂/(CH₃OH/NH₃) (1/1). The solvent was evaporated and the residue was washed with CH₃OH and then collected. Yield: 0.0206 g of final compound 58 (62%).

### Example B5

### Preparation of Final Compound 79

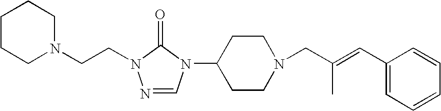

A mixture of intermediate compound 7 ((prepared according to A2.c) (0.00017 mol), 1-(2-chloroethyl)-piperidine hydrochloride (0.00051 mol) and PS-TBD ((2.70 mmol/g) (0.00051 mol) in CH₃CN (2 ml) was heated in a microwave oven for 20 minutes at 130° C., then extra PS-TBD (2.70 mmol/g) (0.00051 mol) was added and the reaction mixture was heated in a microwave oven for 20 minutes at 130° C. The solids were filtered off and washed with CH₂Cl₂. The filtrates solvent was evaporated and the obtained residue was purified by high-performance liquid chromatography. The desire product fractions were collected and the solvent was evaporated. Yield: 0.0069 g of final compound 79 (10%).

### Example B6

### Preparation of Final Compound 38

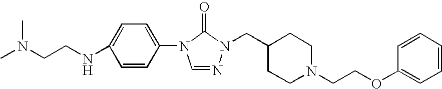

A mixture of intermediate compound 13 (prepared according to A4.a) (0.00011 mol), N,N-dimethyl-1,2-ethanediamine (0.00017 mol), Pd₂(dba)₃ (0.000006 mol), BINAP (0.000017 mol) and t-BuOK (0.00017 mol) in toluene (deoxygenated) (0.5 ml) was heated under microwave irradiation for 15 minutes at 170° C., then the solids were filtered off and washed with CH₂Cl₂. The filtrate's solvent was evaporated and the obtained residue was percolated in a manifold (eluent 1: CH₂Cl₂/CH₃OH 95/5; eluent 2: CH₂Cl₂/(CH₃OH/NH₃) 95/5). The product fractions were collected and the solvent was evaporated. The desired product was caught in a SCX-2 cartridge and was then released with CH₃OH/NH₃. The solvent was evaporated and the obtained residue was lyophilised. Yield: 0.033 g of final compound 38 (65%).

### Example B7

### Preparation of Final Compound 51

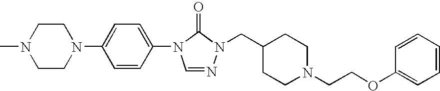

A mixture of intermediate compound 13 (prepared according to A4.a) (0.437 mmol), 1-methylpiperazine (0.65 mmol), Pd(OA)₂ (0.021 mmol), Xantphos (0.0437 mmol), Cs₂CO₃ (0.65 mmol) in Dioxane/DMF 9/1 (2.96 ml) was heated in a microwave oven at 170° C. for 15 minutes. The solid was filtered off over CELITE and the filtrate was evaporated till dryness. The residue was purified by HPLC (gradient CH₃CN/NH₄HCO₃). Selected fractions were collected and their solvent evaporated. The residue was crystallized with diisopropylether. Yield: 82.5 mg of f final compound 51 (40%).

### Example B8

### a. Preparation of Final Compound 28

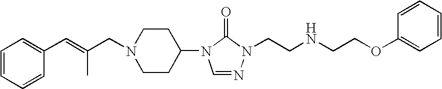

A mixture of intermediate compound 15 (prepared according to A7.g) (0.000078 mol), 2-phenoxyethanamine (0.000156 mol), N,N,N-tributyl-1-butanaminium bromide (0.000078 mol) and K₂CO₃ (0.000156 mol) in CH₃CN (0.5 ml) was heated in a microwave oven for 15 minutes at 120° C. and then resin-linked-CHO (0.000312 mol) and CH₂Cl₂ (1 ml) were added. The reaction mixture was heated in a microwave oven for 20 minutes at 100° C. and the solids were filtered off. The solvent was evaporated and the residue was purified in a manifold (eluent 1: EtOAc; eluent 2: CH₂Cl₂/CH₃OH 96/4). The product fractions were collected and further purified by Catch in an ISOLUTE SCX-3 cartridge and then released with CH₃OH/NH₃. Finally, the desired fractions were purified by high-performance liquid chromatography, then the product fractions were collected and the solvent was evaporated. Yield: 0.0066 g of final compound 28 (18%).

Final compound 8 was made accordingly but the resin-linked-CHO was not added.

### b. Preparation of Final Compound 29

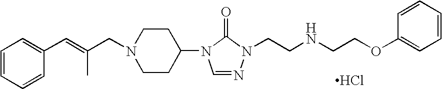

Final compound 28 (prepared according to B8.a) (0.00061 mol) was purified by high-performance liquid chromatography, then the product fractions were collected and precipitated as a HCl-salt (1:2) in EtOH with HCl/2-propanol (q.s.). The solvent was evaporated and the obtained residue was washed with 2-propanone. Yield: 0.129 g of final compound 29 (40%).

### Example B9

### a. Preparation of Final Compound 23

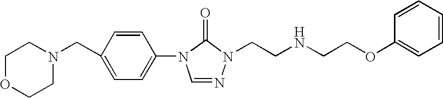

Trifluoroacetic acid (0.5 ml) was added to a mixture of intermediate compound 18 (prepared according to A8.c) (0.00023 mol) in CH₂Cl₂ (2 ml) and the reaction mixture was stirred for 1 hour at room temperature and then a saturated Na₂CO₃ solution was added. The organic layer was separated, dried (Na₂SO₄), filtered off and the solvent was evaporated. The residue was taken up in CH₃OH, then caught in an ISOLUTE SCX-2 cartridge and released with CH₃OH/NH₃. Yield: 0.052 g of final compound 23 (53%).

### b. Preparation of Final Compound 24

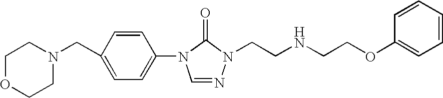

Intermediate compound 18 (prepared according to A8.c) (5.5 mmol) was treated with HCl/isopropanol (35 ml) at room temperature overnight. The solid was collected and washed with absolute ethanol, then dried. Yield: 1.9 g of final compound 24 (70%).

### c. Preparation of Final Compound 21

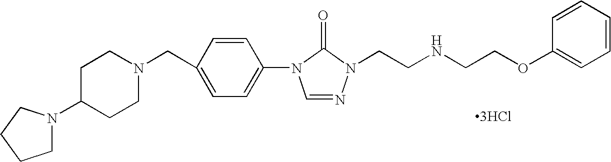

A mixture of intermediate compound 17 (prepared according to A8.b) (0.0008 mol), 4-(1-pyrrolidinyl)piperidine (0.0012 mol) and NaBH(OAc)₃ (0.0012 mol) in 1,2-dichloroethane (10 ml) was heated under microwave irradiation for 10 minutes at 100° C. and then a 37% NH₄OH solution was added. The organic layer was separated, dried (Na₂SO₄) and the solvent was evaporated. The residue was purified in a manifold (vacuum) using a silica cartridge (10 g) (eluent 1: CH₂Cl₂/EtOAc 2/1; eluent 2: CH₂Cl₂/CH₃OH 95/5; eluent 3: CH₂Cl₂/(CH₃OH/NH₃) 95/5->9/1). The product fractions were collected and the solvent was evaporated. The obtained residue was treated with HCl/2-propanol (3 ml) (precipitation) and then a 37% aqueous HCl solution was added. The resulting mixture was stirred for 24 hours at room temperature, then absolute EtOH was added and the solids were collected. Yield: 0.1701 g of final compound 21.

### d. Preparation of Final Compound 93

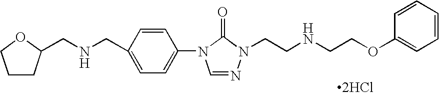

A mixture of intermediate compound 17 (prepared according to A8.b) (0.00011 mol), tetrahydro-2-furanmethanamine (0.00017 mol) and resin-linked-BH(OA)₃ (0.00033 mol; 2.07 mmol/g) in 1,2-dimethoxyethane (1 ml) was heated for 10 minutes at 140° C. under microwave irradiation, then extra tetrahydro-2-furanmethanamine (2×0.0175 ml) and resin-linked-BH(OA)₃ (2×0.159 g) were added and the solids were filtered off. The organic solvent was evaporated and 37% HCl was added to the aqueous concentrate. The reaction mixture was stirred for 24 hours at room temperature and EtOH (3 ml) was added, then the resulting solids were collected and dried. Yield: 0.020 g of final compound 93 (36%).

### Example B10

### Preparation of Final Compound 99

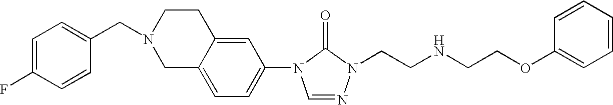

Pd/C 10% (0.5 ml) was added to a mixture of intermediate 34 (prepared according to A10.f)) (0.00023 mol) in 1,4-cyclohexadiene (2 ml) and the reaction mixture was stirred for 1 hour at room temperature. Then the solid was filtered off and the filtrate solvent was treated with a saturated Na₂CO₃ solution. The organic layer was separated, dried (Na₂SO₄), filtered off and the solvent was evaporated. The residue was taken up in CH₃OH, then caught in an ISOLUTE SCX-2 cartridge and released with CH₃OH/NH₃. Yield: 0.052 g of final compound 99 (53%).

### Example B 11

### a. Preparation of Final Compound 9

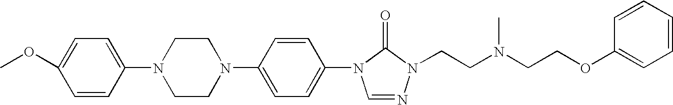

A mixture of final compound 8 (prepared according to B8.a) (0.00020 mol), CHO (37%) (0.00030 mol), NaBH₃CN (0.00030 mol) and ZnBr₂ (0.00010 mol) in CH₃OH (2 ml) was heated in a microwave oven for 5 minutes at 140° C., then a NH₄OH solution (37% NH₃ in H₂O) was added and the mixture was extracted with CH₂Cl₂. The organic layer was separated, dried (Na₂SO₄), filtered off and the solvent was evaporated. The residue was percolated in a manifold using a silica cartridge (5 g) (eluent gradient: CH₂Cl₂/CH₃OH 98/2, 97/3). The product fractions were collected, then washed with ethyl ether and the solvent was evaporated. Yield: 0.054 g of final compound 9 (51%).

### b. Preparation of Final Compound 30

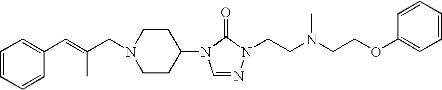

A mixture of final compound 28 (prepared according to B8.a) (0.00022 mol), H₂CO (37%) (0.00033 mol), NaBH₃CN (0.00033 mol) and Zn₂Br₂ (0.00011 mol) in CH₃OH (2 ml) was heated under microwave irradiation for 5 minutes at 140° C. and then for 10 minutes at 150° C. Extra H₂CO (37%) (0.00033 mol) and NaBH₃CN (0.00033 mol) were added and the reaction mixture was heated for 5 minutes at 150° C. The desired product was caught in a SCX-2 cartridge and released with CH₃OH/NH₃, then further purified in a manifold (eluent gradient: CH₂Cl₂/CH₃OH 98/2, 96/4) and purified by high-performance liquid chromatography. The product fractions were collected and the solvent was evaporated. Yield: 0.0371 g of final compound 30 (35%).

### Example B12

### Preparation of Final Compound 4, 5 and 6

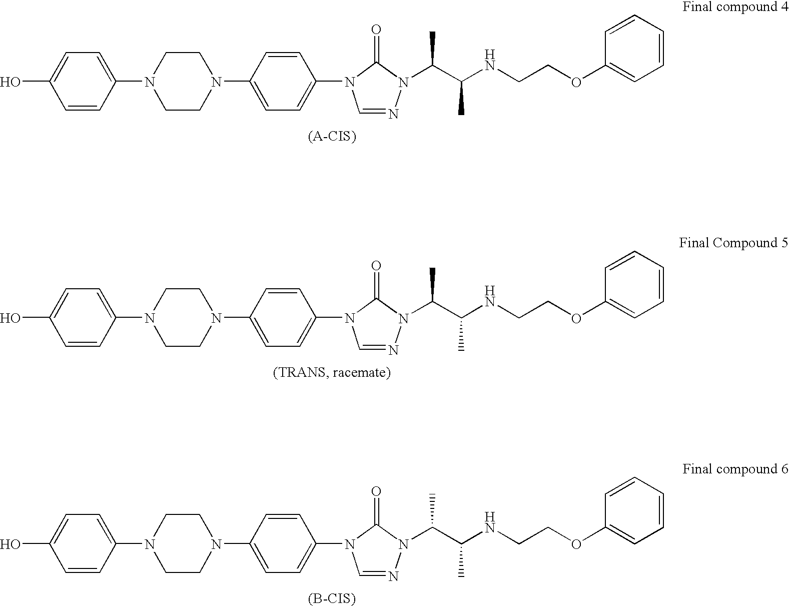

Final compound 7 (prepared according to B3) (q.s.) was separated by Chiral separation (Chiralpak AD) (eluent: CH₃OH 100%). Two fractions were collected. Fraction (I) (Mixture of compounds 4 and 5) and Fraction (II). Yield Fraction (II): 0.119 g of final compound 6 (enantiomer B-CIS). Fraction (I) was further separated by Chiral separation (Chiralpak AD) (eluent: EtOH/Heptane 70/30) and then two product fractions were collected. Yield Fraction (III): 0.110 g of final compound 4 (enantiomer A-CIS). Yield Fraction (IV): 0.260 g final compound 5 (TRANS as racemate).

### Example B13

### Preparation of Final Compound 120

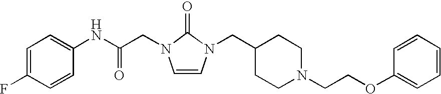

HATU (0.00068 mol) was added to a mixture of intermediate compound 54 (prepared according to A16.d) (0.00052 mol), 1-methylpiperazine (0.00047 mol) and N-(1,1-dimethylethyl)-N-ethyl-2-methyl-2-propanamine (0.00068 mol) in CH₂Cl₂ (10 ml) and DMF (2.5 ml). The reaction mixture was stirred for 4 hours at room temperature. Water was added. This mixture was extracted with CH₂Cl₂. The separated organic layer was dried (Na₂SO₄), filtered and the solvent evaporated. The residue was purified by column chromatography in a Manifold (10 g silica gel cartridge; eluent: CH₂Cl₂/CH₃OH 95/5; then CH₂Cl₂/(CH₃OH/NH₃) 95/5 and 90/10). The product fractions were collected and the solvent was evaporated. The residue was washed with diethyl ether, then dried. Yield: 0.1375 g of final compound 120 (58%).

### Example B14

### Preparation of Final Compound 116

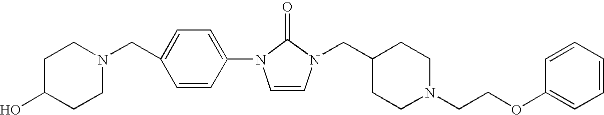

Reaction in microwave. A mixture of intermediate compound

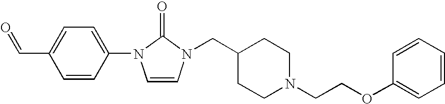

(prepared according to A15.a) (0.0005 mol), 4-piperidinol (0.00075 mol) and NaBH(OAc)₃ (0.00075 mol) in 1,2-dichloroethane (2 ml) was heated for 15 minutes at 80° C. A 32% aqueous NH₃ solution was added. The organic layer was separated, dried (Na₂SO₄), filtered and the solvent was evaporated. The residue was purified by column chromatography in a Manifold (eluent: CH₂Cl₂/CH₃OH 95/5, then CH₂Cl₂/(CH₃OH/NH₃) 95/5, then 90/10). The product fractions were collected and the solvent was evaporated. The residue was dissolved in 2-propanol and converted into the hydrochloric acid salt (1:2) with HCl/2-propanol. The precipitate was filtered off and dried. Yield: 0.018 g of final compound 116.

### Example B15

### Preparation of Final Compound 106

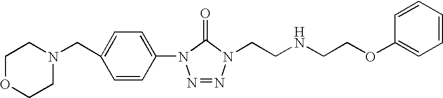

An intermediate compound

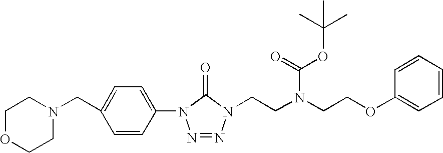

(prepared according to A15.b) (0.0003 mol) in HCl/2-propanol (2 ml) was stirred for 24 hours at room temperature. The precipitate was filtered off and dried. Yield: 0.063 g of final compound 106.

Tables 2 to 8 list the compounds of Formula (I) which were prepared according to one of the above described examples.

TABLE 2

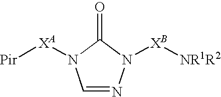

| Co. | Ex. | | | |
|---|---|---|---|---|
| Nr. | Nr | Pir | X^{A} | X^{B} |
| 1 | B9.c | 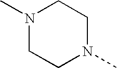 | 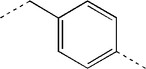 | 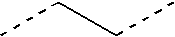 |
| 2 | B9.c | 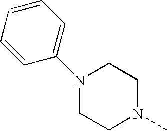 | 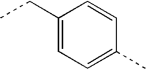 | 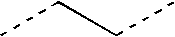 |
| 3 | B9.c | 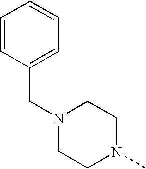 | 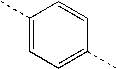 | 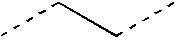 |
| 4 | B12 | 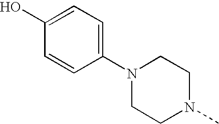 | 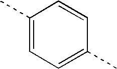 | 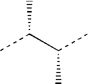 |
| 5 | B12 | 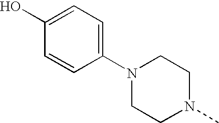 | 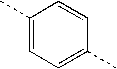 | 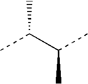 |
| 6 | B12 | 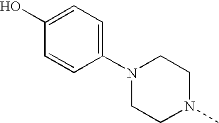 | 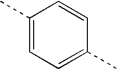 | 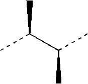 |
| 7 | B3 | 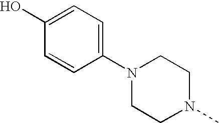 | 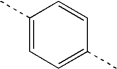 | 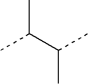 |
| 8 | B8.a | 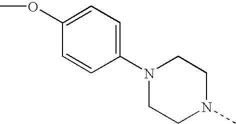 | 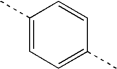 | 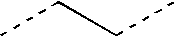 |
| 9 | B11.a | 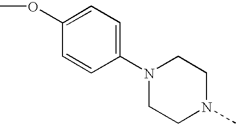 | 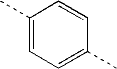 | 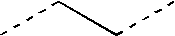 |
| 10 | B3 | 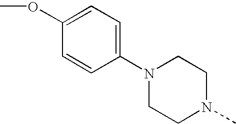 | 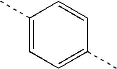 | 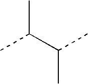 |
| 11 | B1.b | 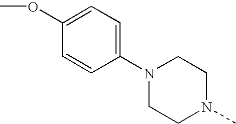 | 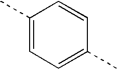 | 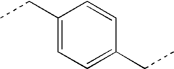 |
| 12 | B7 | 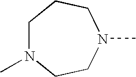 | 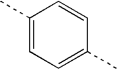 | 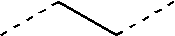 |
| 13 | B9.c | 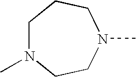 | 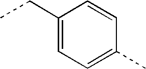 | 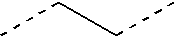 |
| 14 | B9.c | 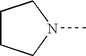 | 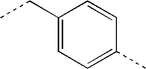 | 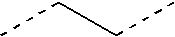 |
| 15 | B7 | 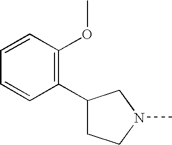 | 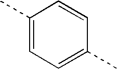 | 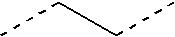 |
| 16 | B5 | 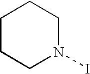 | 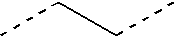 | 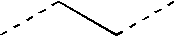 |
| 17 | B9.c | 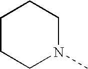 | 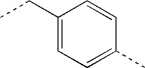 | 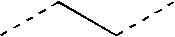 |
| 18 | B9.a | 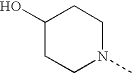 | 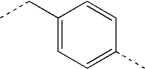 | 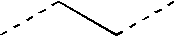 |
| 19 | B9.d | 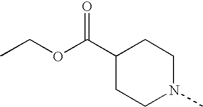 | 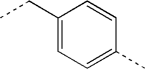 | 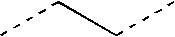 |
| 20 | B7 | 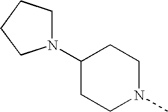 | 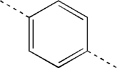 | 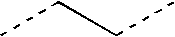 |
| 21 | B9.c | 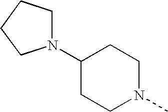 | 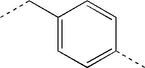 | 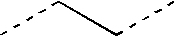 |
| 22 | B7 | 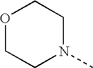 | 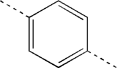 | 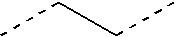 |
| 23 | B9.a | 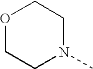 | 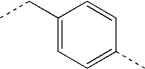 | 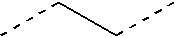 |
| 24 | B9.b | 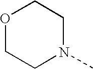 | 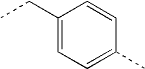 | 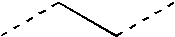 |
| 25 | B9.c | 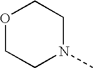 | 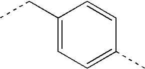 | 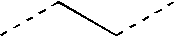 |
| 26 | B9.c | 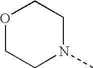 | 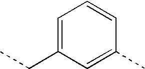 | 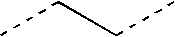 |
| 27 | B9.c | 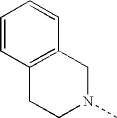 | 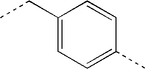 | 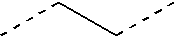 |
| 28 | B8.a | 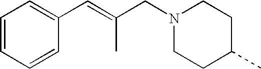 | — | 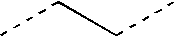 |
| 29 | B8.b | 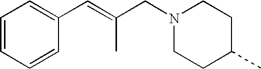 | — | 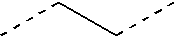 |
| 30 | B11.b | 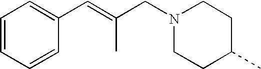 | — | 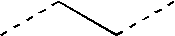 |
| 31 | B6 | 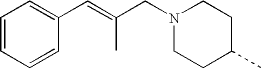 | — | 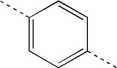 |
| 32 | B1.6 | 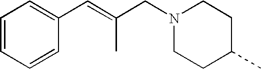 | — | 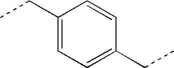 |
| 33 | B1.b | 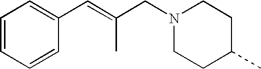 | — | 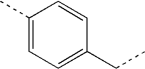 |

| Co. | Ex. | | | Stereo-chemistry + |
|---|---|---|---|---|
| Nr. | Nr | R¹ | R² | salt |
| 1 | B9.c | H | 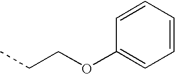 | •HCl |
| 2 | B9.c | H | 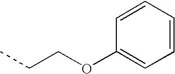 | •HCl |
| 3 | B9.c | H | 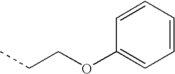 | •CF₃COOH |
| 4 | B12 | H | 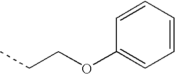 | A-cis |
| 5 | B12 | H | 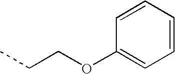 | trans-racemate |
| 6 | B12 | H | 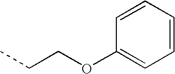 | B-cis |
| 7 | B3 | H | 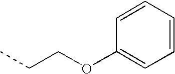 | mixture of enantiomers |
| 8 | B8.a | H | 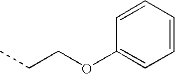 | |
| 9 | B11.a | CH₃ | 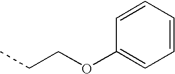 | |
| 10 | B3 | H | 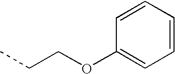 | mixture of diastereoisomers 63% and 37% |
| 11 | B1.b | H | H | |
| 12 | B7 | H | 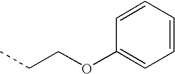 | •CF₃COOH |
| 13 | B9.c | H | 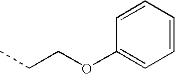 | •HCl |
| 14 | B9.c | H | 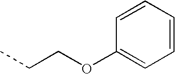 | •HCl |
| 15 | B7 | H | 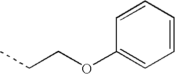 | racemate (for the phenyl-pyrrolidinyl) |
| 16 | B5 | H | 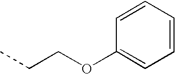 | •HCl |
| 17 | B9.c | H | 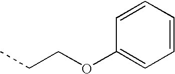 | •HCl |
| 18 | B9.a | H | 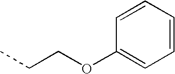 | •HCl |
| 19 | B9.d | H | 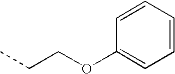 | •HCl |
| 20 | B7 | H | 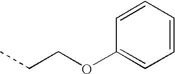 | •CF₃COOH |
| 21 | B9.c | H | 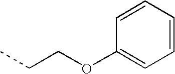 | •HCl |
| 22 | B7 | H | 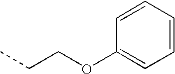 | •CF₃COOH |
| 23 | B9.a | H | 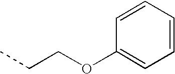 | |
| 24 | B9.b | H | 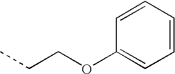 | •HCl |
| 25 | B9.c | H | 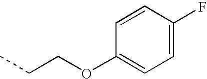 | •HCl |
| 26 | B9.c | H | 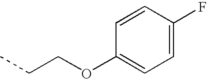 | •HCl |
| 27 | B9.c | H | 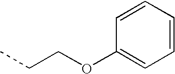 | •HCl |
| 28 | B8.a | H | 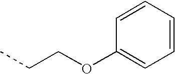 | E (for the cinnamyl) |
| 29 | B8.b | H | 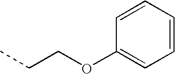 | •HCl; E (for the cinnamyl) |
| 30 | B11.b | CH₃ | 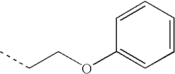 | E (for the cinnamyl) |
| 31 | B6 | H | 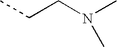 | E (for the cinnamyl) |
| 32 | B1.b | H | H | E (for the cinnamyl) |
| 33 | B1.b | H | H | E (for the cinnamyl) |

TABLE 3

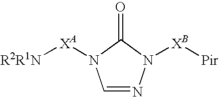

| Co. | Ex. | | | | |
|---|---|---|---|---|---|
| Nr. | Nr | R¹ | R² | X^{A} | X^{B} |
| 34 | B1.b | H | H | 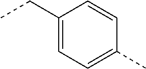 | — |
| 36 | B1.b | H | H | 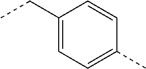 | — |
| 37 | B4.a | H | H | 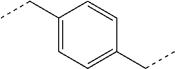 | — |
| 35 | B9.a | H | H | 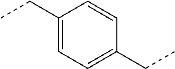 | 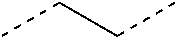 |
| 38 | B6 | 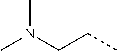 | H | 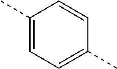 | —CH₂— |
| 39 | B6 | 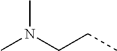 | H | 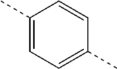 | —CH₂— |
| 40 | B6 | 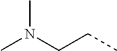 | H | 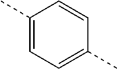 | 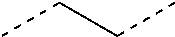 |
| 41 | B6 | 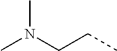 | H | 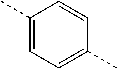 | 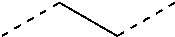 |
| 42 | B6 | 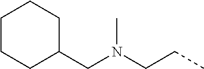 | H | 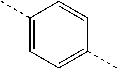 | —CH₂— |
| 43 | B6 | 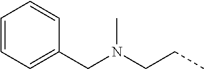 | H | 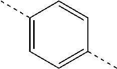 | —CH₂— |
| 44 | B5 | 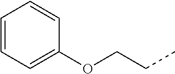 | H | 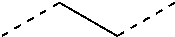 | 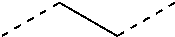 |
| 45 | B2.b | 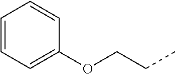 | H | 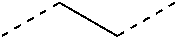 | 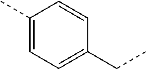 |
| 46 | B4.b | 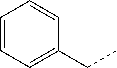 | CH₃ | 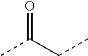 | —CH₂— |
| 47 | B4.b | 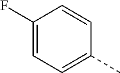 | H | 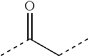 | —CH₂— |
| 48 | B6 | 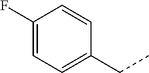 | H | 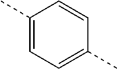 | —CH₂— |
| 49 | B6 | 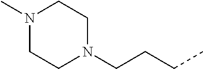 | H | 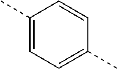 | —CH₂— |
| 50 | B6 | 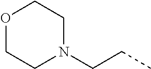 | H | 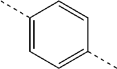 | —CH₂— |

| Co. | | Stereochemistry + |
|---|---|---|
| Nr. | Pir | salt |
| 34 | 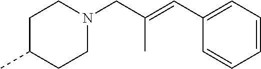 | E (for the cinnamyl) |
| 36 | 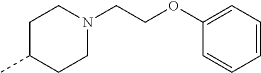 | E (for the cinnamyl) |
| 37 | 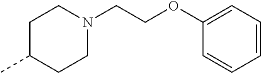 | |
| 35 | 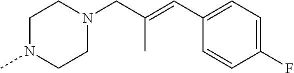 | E (for the cinnamyl) |
| 38 | 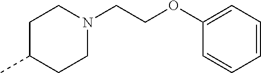 | |
| 39 | 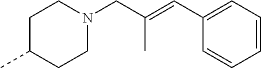 | E (for the cinnamyl) |
| 40 | 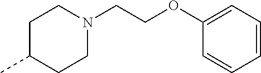 | •HCl |
| 41 | 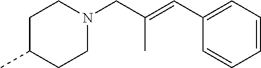 | •HCl; E (for the cinnamyl) |
| 42 | 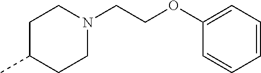 | |
| 43 | 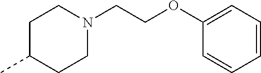 | •HCl |
| 44 | 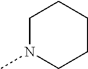 | •HCl |
| 45 | 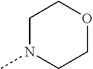 | •HCl |
| 46 | 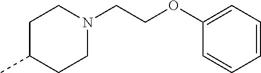 | •HCl |
| 47 | 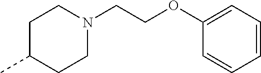 | •HCl |
| 48 | 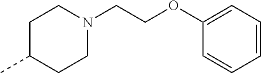 | |
| 49 | 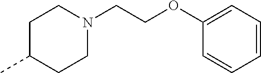 | |
| 50 | 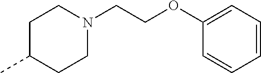 | |

TABLE 4

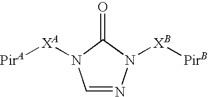

| Co. | Ex. | | | |
|---|---|---|---|---|
| Nr. | Nr | Pir^{A} | X^{A} | X^{B} |
| 51 | B7 | 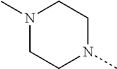 | 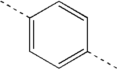 | —CH₂— |
| 52 | B7 | 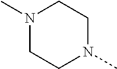 | 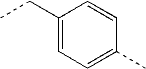 | —CH₂— |
| 53 | B7 | 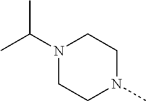 | 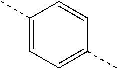 | —CH₂— |
| 54 | B7 | 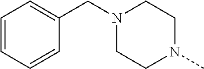 | 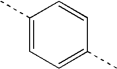 | —CH₂— |
| 55 | B3 | 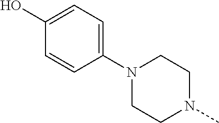 | 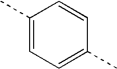 | 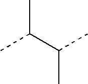 |
| 56 | B2.a | 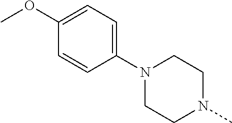 | 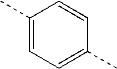 | — |
| 57 | B2.a | 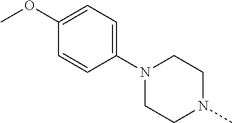 | 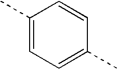 | — |
| 58 | B4.b | 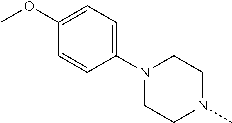 | 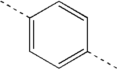 | — |
| 59 | B2.b | 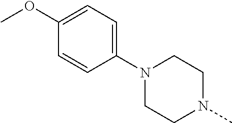 | 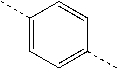 | 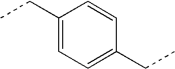 |
| 112 | B1.a | 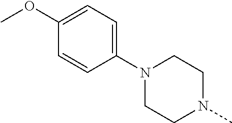 | 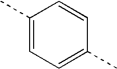 | 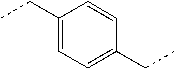 |
| 60 | B7 | 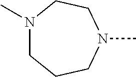 | 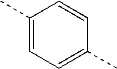 | —CH₂— |
| 61 | B9.c | 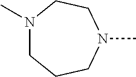 | 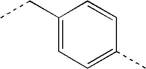 | —CH₂— |
| 62 | B9.c | 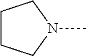 | 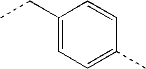 | —CH₂— |
| 63 | B7 | 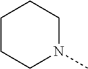 | 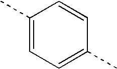 | —CH₂— |
| 64 | B9.c | 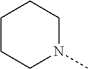 | 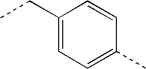 | —CH₂— |
| 65 | B9.c | 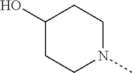 | 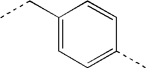 | —CH₂— |
| 66 | B5 | 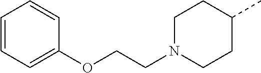 | —CH₂— | 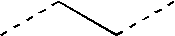 |
| 123 | B5 | 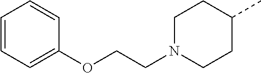 | —CH₂— | 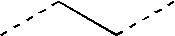 |
| 67 | B5 | 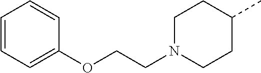 | —CH₂— | 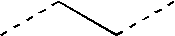 |
| 124 | B5 | 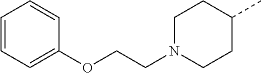 | —CH₂— | 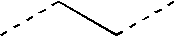 |
| 125 | B5 | 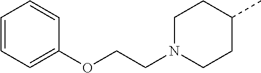 | —CH₂— | 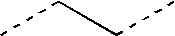 |
| 126 | B5 | 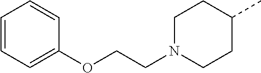 | —CH₂— | 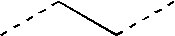 |
| 127 | B5 | 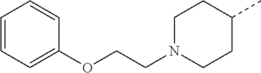 | —CH₂— | 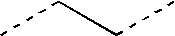 |
| 128 | B5 | 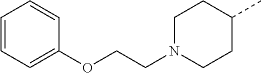 | —CH₂— | 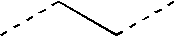 |
| 129 | B5 | 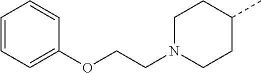 | —CH₂— | 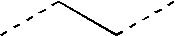 |
| 130 | B5 | 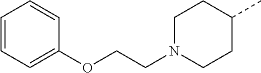 | —CH₂— | 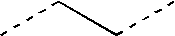 |
| 131 | B5 | 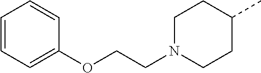 | —CH₂— | 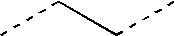 |
| 82 | B5 | 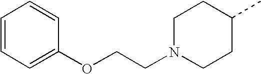 | —CH₂— | 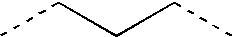 |
| 68 | B9.c | 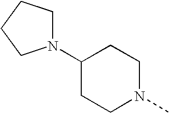 | 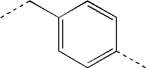 | —CH₂— |
| 69 | B2.b | 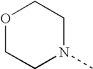 | 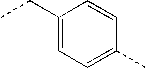 | — |
| 70 | B2.b | 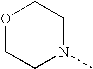 | 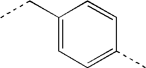 | —CH₂— |
| 71 | B4.b | 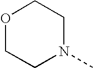 | 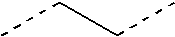 | —CH₂— |
| 72 | B6 | 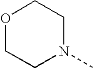 | 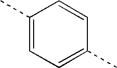 | —CH₂— |
| 73 | B4.b | 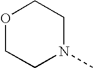 | 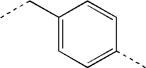 | —CH₂— |
| 74 | B2.a | 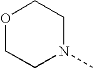 | 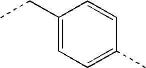 | —CH₂— |
| 75 | B2.a | 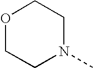 | 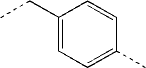 | —CH₂— |
| 76 | B2.a | 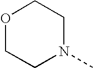 | 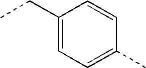 | —CH₂— |
| 77 | B2.a | 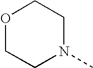 | 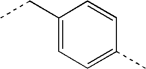 | —CH₂— |
| 78 | B8.a | 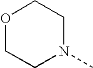 | 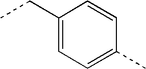 | 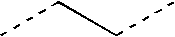 |
| 79 | B5 | 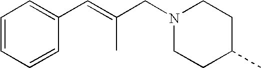 | — | 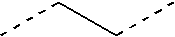 |
| 83 | B5 | 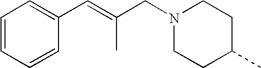 | — | 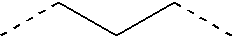 |
| 80 | B2.b | 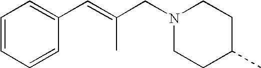 | — | 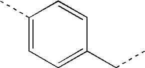 |
| 110 | B1.a | 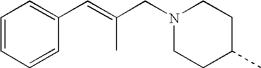 | — | 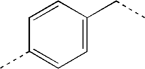 |
| 81 | B2.b | 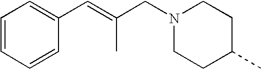 | — | 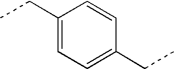 |
| 111 | B1.a | 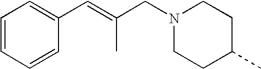 | — | 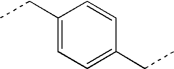 |
| 118 | B4.b | 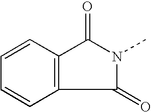 | 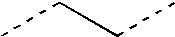 | —CH₂— |
| 84 | B4.b | 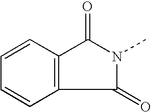 | 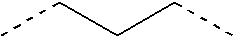 | —CH₂— |
| 109 | B1.a | 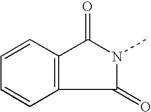 | 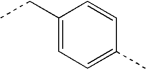 | — |
| 113 | B1.a | 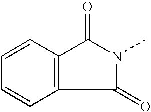 | 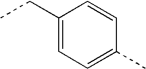 | — |

| Co. | | Stereochemistry + |
|---|---|---|
| Nr. | Pir^{B} | salt |
| 51 | 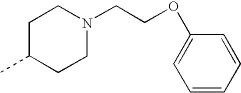 | |
| 52 | 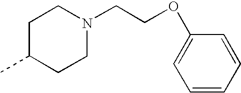 | |
| 53 | 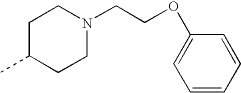 | |
| 54 | 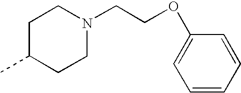 | |
| 55 | 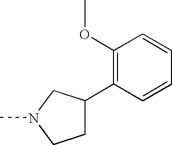 | |
| 56 | 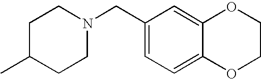 | |
| 57 | 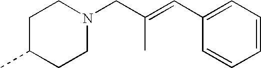 | E (for the cinnamyl) |
| 58 | 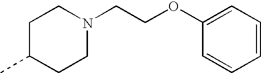 | |
| 59 | 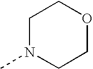 | |
| 112 | 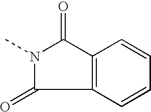 | |
| 60 | 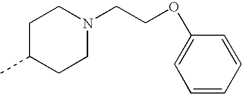 | |
| 61 | 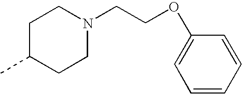 | |
| 62 | 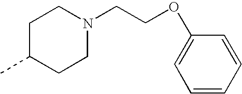 | |
| 63 | 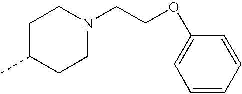 | |
| 64 | 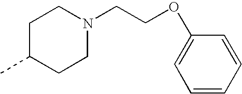 | |
| 65 | 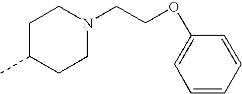 | |
| 66 | 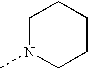 | •CF₃COOH |
| 123 | 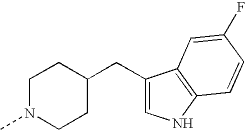 | |
| 67 | 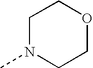 | •CF₃COOH |
| 124 | 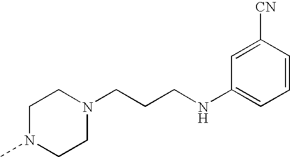 | |
| 125 | 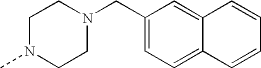 | |
| 126 | 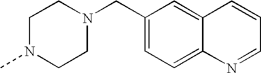 | |
| 127 | 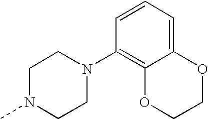 | |
| 128 | 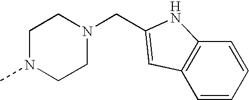 | |
| 129 | 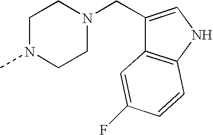 | |
| 130 | 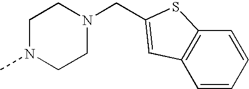 | |
| 131 | 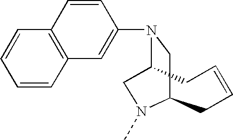 | |
| 82 | 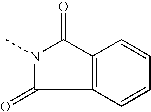 | •CF₃COOH |
| 68 | 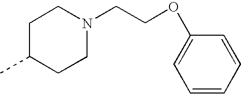 | |
| 69 | 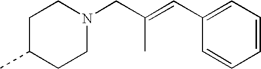 | E (for the cinnamyl) |
| 70 | 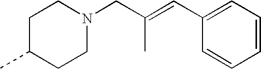 | E (for the cinnamyl) |
| 71 | 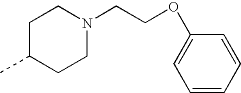 | •HCl |
| 72 | 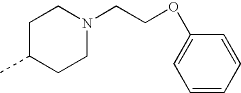 | |
| 73 | 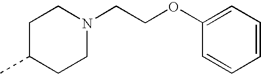 | |
| 74 | 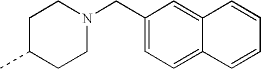 | |
| 75 | 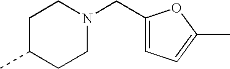 | |
| 76 | 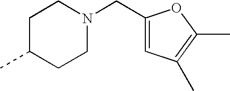 | |
| 77 | 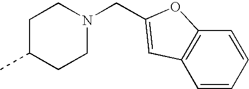 | |
| 78 | 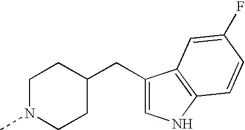 | |
| 79 | 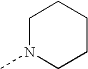 | E (for the cinnamyl) |
| 83 | 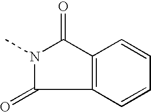 | E (for the cinnamyl) |
| 80 | 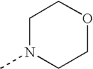 | E (for the cinnamyl) |
| 110 | 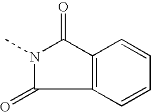 | E (for the cinnamyl) |
| 81 | 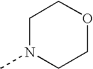 | E (for the cinnamyl) |
| 111 | 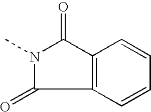 | E (for the cinnamyl) |
| 118 | 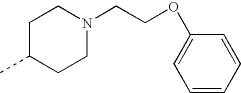 | •HCl |
| 84 | 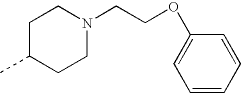 | •HCl |
| 109 | 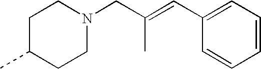 | E (for the cinnamyl) |
| 113 | 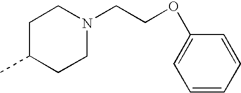 | |

TABLE 5

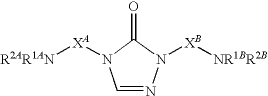

| Co. | Ex. | | | | | | | |
|---|---|---|---|---|---|---|---|---|
| Nr. | Nr | R^{1A} | R^{2A} | X^{A} | X^{B} | R^{1B} | R^{2B} | Salt |
| 85 | B1.b | H | H | 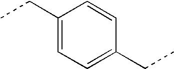 | 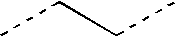 | H | 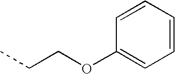 | |
| 86 | B6 | H | 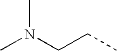 | 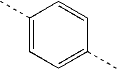 | 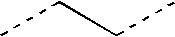 | H | 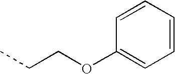 | |
| 87 | B6 | H | 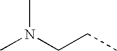 | 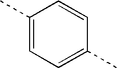 | 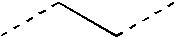 | H | 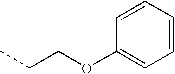 | •HCl |
| 88 | B6 | H | 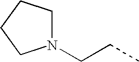 | 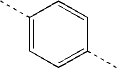 | 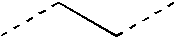 | H | 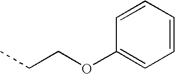 | •CF₃COOH |
| 89 | B6 | H | 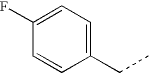 | 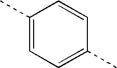 | 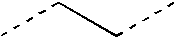 | H | 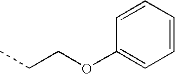 | •CF₃COOH |
| 90 | B9.d | H | 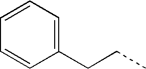 | 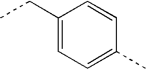 | 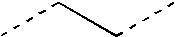 | H | 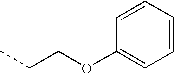 | •HCl |
| 91 | B9.d | H | 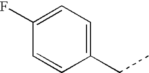 | 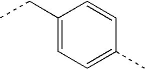 | 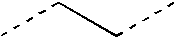 | H | 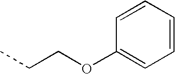 | •HCl |
| 92 | B9.d | H | 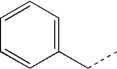 | 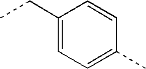 | 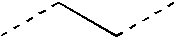 | H | 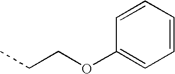 | •HCl |
| 93 | B9.d | H | 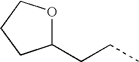 | 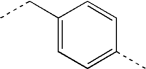 | 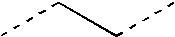 | H | 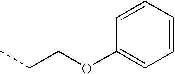 | •HCl |
| 94 | B9.d | CH₃ | 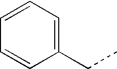 | 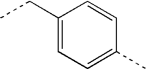 | 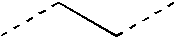 | H | 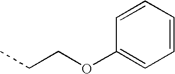 | •HCl |
| 95 | B9.d | H | 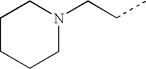 | 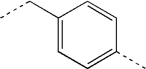 | 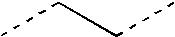 | H | 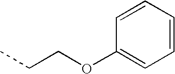 | •HCl |
| 96 | B9.d | H | 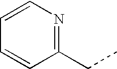 | 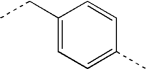 | 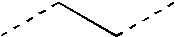 | H | 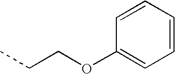 | •HCl |

TABLE 6

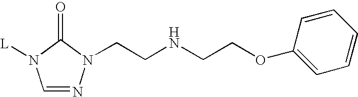

| Co. | Ex. | | |
|---|---|---|---|
| Nr. | Nr | L | Salt |
| 97 | B10 | 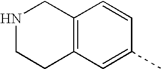 | •HCl |
| 98 | B10 | 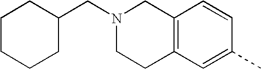 | |
| 99 | B10 | 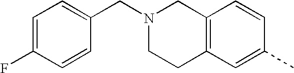 | |
| 100 | B10 | 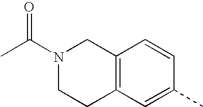 | •HCl |
| 101 | B10 | 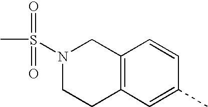 | |
| 102 | B10 | 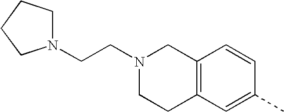 | |
| 103 | B10 | 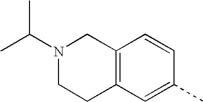 | |

TABLE 7

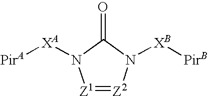

| Co. | Ex. | | | | | | | |
|---|---|---|---|---|---|---|---|---|
| Nr. | Nr | Pir^{A} | X^{A} | Z¹ | Z² | X^{B} | Pir^{B} | Salt |
| 104 | B2.b | 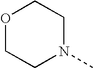 | 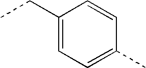 | N | N | —CH₂— | 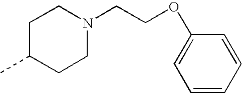 | |
| 105 | B14 | 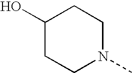 | 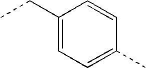 | N | N | —CH₂— | 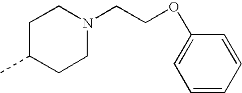 | •HCl |
| 108 | B7 | 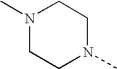 | 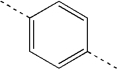 | N | N | —CH₂— | 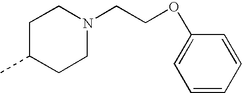 | •HCl |
| 115 | B14 | 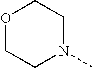 | 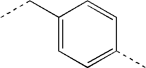 | C | C | —CH₂— | 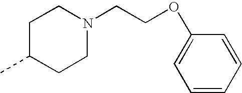 | •HCl |
| 116 | B14 | 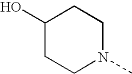 | 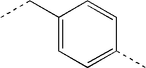 | C | C | —CH₂— | 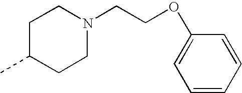 | •HCl |
| 121 | B7 | 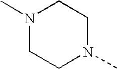 | 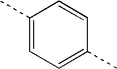 | C | C | —CH₂— | 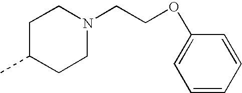 | •HCl |

TABLE 8

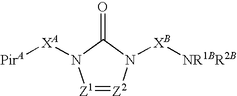

| Co. | Ex. | | | | | | | | |
|---|---|---|---|---|---|---|---|---|---|
| Nr. | Nr | Pir^{A} | X^{A} | Z¹ | Z² | X^{B} | R^{1B} | R^{2B} | Salt |
| 106 | B15 | 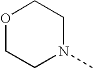 | 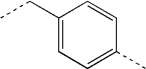 | N | N | —CH₂—CH₂— | H | 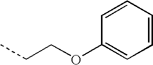 | •HCl |
| 107 | B15 | 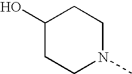 | 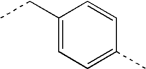 | N | N | —CH₂—CH₂— | H | 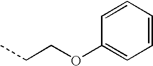 | •HCl |
| 114 | B15 | 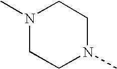 | 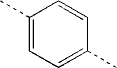 | N | N | —CH₂—CH₂— | H | 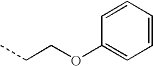 | •HCl |
| 117 | B14 | 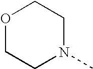 | 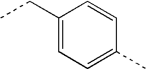 | C | C | —CH₂—CH₂— | H | 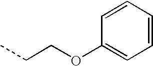 | •HCl |
| 119 | B14 | 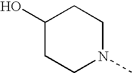 | 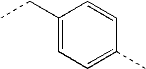 | C | C | —CH₂—CH₂— | H | 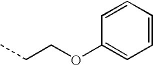 | •HCl |
| 122 | B14 | 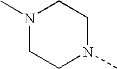 | 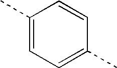 | C | C | —CH₂—CH₂— | H | 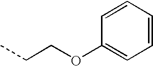 | •HCl |
| 120 | B13 | 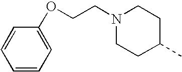 | —CH₂— | C | C | 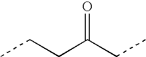 | H | 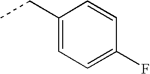 | |

TABLE 9

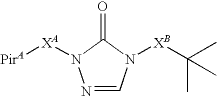

| Co. Nr. | Ex. Nr | Pir^{A} | X^{A} | X^{B} | Salt |
|---|---|---|---|---|---|
| 132 | B4.a | 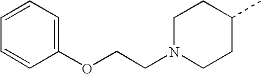 | —CH₂— | 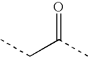 | |

### C. Pharmacological Example

### General

The interaction of the compounds of Formula (I) with α_{2C}-adrenoceptor receptors was assessed in in vitro radioligand binding experiments. In general, a low concentration of a radioligand with a high binding affinity for a particular receptor or transporter is incubated with a sample of a tissue preparation enriched in a particular receptor or transporter or with a preparation of cells expressing cloned human receptors in a buffered medium. During the incubation, the radioligand binds to the receptor or transporter. When equilibrium of binding is reached, the receptor bound radioactivity is separated from the non-bound radioactivity, and the receptor- or transporter-bound activity is counted. The interaction of the test compounds with the receptor is assessed in competition binding experiments. Various concentrations of the test compound are added to the incubation mixture containing the receptor- or transporter preparation and the radioligand. The test compound in proportion to its binding affinity and its concentration inhibits binding of the radioligand. The radioligand used for hα_{2C}, hα_{2C} and hα_{2C} receptor binding was [³H]-raulwolscine.

### Example C.1

### Binding Experiment for α_{2C}-adrenoceptor

### Cell Culture and Membrane Preparation

CHO cells, stabile transfected with human adrenergic-α_{2A}-, -α_{2B} or α_{2C} receptor cDNA, were cultured in Dulbecco's Modified Eagle's Medium (DMEM)/Nutrient mixture Ham's F12 (ratio 1:1)(Gibco, Gent-Belgium) supplemented with 10% heat inactivated fetal calf serum (Life Technologies, Merelbeke-Belgium) and antibiotics (100 IU/ml penicillin G, 100 μg/ml streptomycin sulphate, 110 μg/ml pyruvic acid and 100 μg/ml L-glutamine). One day before collection, cells were induced with 5 mM sodiumbutyrate. Upon 80-90% of confluence, cells were scraped in phosphate buffered saline without Ca²⁺ and Mg²⁺ and collected by centrifugation at 1500×g for 10 min. The cells were homogenised in Tris-HCl 50 mM using an Ultraturrax homogenizer and centrifuged for 10 min at 23,500×g. The pellet was washed once by resuspension and rehomogenization and the final pellet was resuspended in Tris-HCl, divided in 1 ml aliquots and stored at −70° C.

Binding Experiment for α₂-adrenergic Receptor Subtypes

Membranes were thawed and re-homogenized in incubation buffer (glycylglycine 25 mM, pH 8.0). In a total volume of 500 μl, 2-10 μg protein was incubated with [³H]raulwolscine (NET-722) (New England Nuclear, USA) (1 nM final concentration) with or without competitor for 60 min at 25° C. followed by rapid filtration over GF/B filter using a Filtermate196 harvester (Packard, Meriden, Conn.). Filters were rinsed extensively with ice-cold rinsing buffer (Tris-HCl 50 mM pH 7.4). Filter-bound radioactivity was determined by scintillation counting in a Topcount (Packard, Meriden, CT) and results were expressed as counts per minute (cpm). Non-specific binding was determined in the presence of 1 μM oxymetazoline for hα_{2A}- and hα_{2B} receptors and 1 μM spiroxatrine for hα_{2C} receptors.

### Data Analysis and Results

Data from assays in the presence of compound were calculated as a percentage of total binding measured in the absence of test compound. Inhibition curves, plotting percent of total binding versus the log value of the concentration of the test compound, were automatically generated, and sigmoidal inhibition curves were fitted using non-linear regression. The pIC₅₀ values of test compounds were derived from individual curves.

All compounds according to Formula (I) produced an inhibition at least at the hα_{2C} site (but often also at the hα_{2A} and hα_{2B}-sites) of more than 50% (pIC₅₀) at a test concentration ranging between 10⁻⁶ M and 10⁻⁹ M in a concentration-dependent manner.

For a selected number of compounds, covering most of the various embodiments of Formula (I), the results of the in vitro studies are given in Table 8.

TABLE 8
Pharmacological data for the compounds according to the invention.

| | pIC₅₀ | | |
|---|---|---|---|
| Co. No. | α_{2A} | α_{2B} | α_{2C} |
| 62 | 7.4 | 8.0 | 9.3 |
| 64 | 7.3 | 7.9 | 9.2 |
| 84 | 7.6 | 7.7 | 9.2 |
| 90 | 7.7 | 7.3 | 9.2 |
| 40 | 8.4 | 9.2 | 9.1 |
| 17 | 7.5 | 7.2 | 9.1 |
| 42 | 7.6 | 8.1 | 9.0 |
| 82 | 7.9 | 7.8 | 9.0 |
| 86 | 7.4 | 7.5 | 9.0 |
| 13 | 7.2 | 7.2 | 9.0 |
| 27 | 7.6 | 7.1 | 9.0 |
| 14 | 7.3 | 7.1 | 9.0 |
| 61 | 7.2 | 8.0 | 8.9 |
| 65 | 7.1 | 7.7 | 8.9 |
| 87 | 7.5 | 7.5 | 8.9 |
| 93 | 7.3 | 6.9 | 8.9 |
| 41 | 8.2 | 8.9 | 8.8 |
| 38 | 7.6 | 8.4 | 8.8 |
| 43 | 7.6 | 8.2 | 8.8 |
| 124 | 7.2 | n.d. | 8.8 |
| 73 | 6.8 | 7.4 | 8.8 |
| 1 | 7.0 | 7.2 | 8.8 |
| 21 | 7.4 | 7.1 | 8.8 |
| 12 | 6.9 | 7.0 | 8.8 |
| 95 | 7.8 | 6.9 | 8.8 |
| 18 | 7.3 | 6.9 | 8.8 |
| 60 | 7.4 | 8.3 | 8.7 |
| 129 | 7.3 | n.d. | 8.7 |
| 109 | 7.5 | 7.4 | 8.7 |
| 88 | 7.4 | 7.4 | 8.7 |
| 89 | 6.6 | 6.9 | 8.7 |
| 97 | 7.3 | 6.6 | 8.7 |
| 125 | 7.5 | n.d. | 8.6 |
| 68 | 7.4 | 8.1 | 8.6 |
| 66 | 7.4 | 7.3 | 8.6 |
| 130 | 7.3 | n.d. | 8.6 |
| 48 | 6.7 | 7.6 | 8.5 |
| 94 | 7.5 | 6.9 | 8.5 |
| 96 | 7.4 | 6.7 | 8.5 |
| 19 | 7.2 | 6.6 | 8.5 |
| 102 | 7.1 | 6.6 | 8.5 |
| 23 | 6.8 | 6.6 | 8.5 |
| 127 | 7.2 | n.d. | 8.4 |
| 49 | 7.2 | 8.2 | 8.4 |
| 37 | 7.4 | 7.6 | 8.4 |
| 47 | 7.1 | 7.4 | 8.4 |
| 20 | 7.0 | 7.0 | 8.4 |
| 29 | 7.0 | 6.9 | 8.4 |
| 28 | 6.9 | 6.7 | 8.4 |
| 132 | 7.2 | n.d. | 8.3 |
| 53 | 7.0 | 8.4 | 8.3 |
| 52 | 7.0 | 7.7 | 8.3 |
| 63 | 6.8 | 7.5 | 8.3 |
| 83 | 6.5 | 7.3 | 8.3 |
| 91 | 7.5 | 7.1 | 8.3 |
| 104 | 7.2 | 7.1 | 8.3 |
| 92 | 7.6 | 6.7 | 8.3 |
| 24 | 6.6 | 6.6 | 8.3 |
| 85 | 6.9 | 6.9 | 8.2 |
| 46 | 6.9 | 6.8 | 8.2 |
| 4 | 5.6 | 6.7 | 8.2 |
| 125 | 7.4 | n.d. | 8.1 |
| 131 | 6.9 | n.d. | 8.1 |
| 51 | 6.8 | 8.1 | 8.1 |
| 72 | 6.8 | 7.6 | 8.1 |
| 7 | 6.9 | 6.9 | 8.1 |
| 101 | 6.2 | 6.6 | 8.1 |
| 39 | 7.2 | 8.1 | 8.0 |
| 34 | 7.4 | 7.3 | 8.0 |
| 99 | 6.6 | 6.7 | 8.0 |
| 100 | 6.2 | 6.4 | 8.0 |
| 5 | 6.3 | 6.3 | 8.0 |
| 16 | 6.8 | 5.8 | 8.0 |
| 44 | 6.9 | 5.4 | 8.0 |
| 128 | 7.1 | n.d. | 7.9 |
| 126 | 6.7 | n.d. | 7.9 |
| 50 | 6.6 | 7.8 | 7.9 |
| 31 | 7.3 | 7.6 | 7.9 |
| 32 | 6.9 | 7.3 | 7.9 |
| 36 | 7.4 | 7.1 | 7.9 |
| 108 | 6.1 | 6.6 | 7.9 |
| 54 | 6.8 | 7.5 | 7.8 |
| 55 | 5.9 | 7.3 | 7.8 |
| 33 | 7.8 | 7.2 | 7.8 |
| 8 | 6.2 | 6.8 | 7.8 |
| 30 | 6.4 | 6.7 | 7.8 |
| 3 | 6.4 | 6.6 | 7.8 |
| 10 | 6.2 | 6.3 | 7.8 |
| 45 | 6.5 | 6.7 | 7.7 |
| 71 | 6.6 | 6.6 | 7.7 |
| 98 | 6.3 | 6.6 | 7.7 |
| 107 | 6.3 | 6.4 | 7.7 |
| 67 | 6.4 | 7.0 | 7.6 |
| 2 | 6.7 | 6.7 | 7.6 |
| 103 | 6.3 | 6.1 | 7.6 |
| 69 | 6.8 | n.d. | 7.6 |
| 79 | 6.4 | 6.2 | 7.5 |
| 80 | 7.2 | n.d. | 7.4 |
| 70 | 6.4 | n.d. | 7.4 |
| 106 | 6.4 | n.d. | 7.4 |
| 35 | 6.9 | n.d. | 7.3 |
| 22 | 6.1 | n.d. | 7.3 |
| 58 | 5.9 | n.d. | 7.3 |
| 25 | 5.6 | n.d. | 7.3 |
| 105 | 6.5 | n.d. | 7.2 |
| 81 | 6.2 | n.d. | 7.1 |
| 57 | <5 | n.d. | 7.1 |
| 15 | 6.1 | n.d. | 7.0 |
| 74 | 6.1 | n.d. | 6.8 |
| 6 | 5.1 | n.d. | 6.7 |
| 77 | 5.5 | n.d. | 6.6 |
| 59 | 5.8 | n.d. | 6.2 |
| 11 | 5.1 | n.d. | 6.1 |
| 9 | <5 | n.d. | 6.1 |
| 75 | <5 | n.d. | 6.1 |
| 76 | <5 | n.d. | 6.1 |
| 56 | <5 | n.d. | 6.0 |

(n.d. = not determined).

### D. Composition Examples

“Active ingredient” (a.i.) as used throughout these examples relates to a compound of formula (I), the pharmaceutically acceptable acid or base addition salts thereof, the stereochemically isomeric forms thereof, the N-oxide form thereof, a quaternary ammonium salt thereof and prodrugs thereof.

### Example D.1

### Oral Drops

500 Grams of the a.i. is dissolved in 0.5 l of 2-hydroxypropanoic acid and 1.5 l of the polyethylene glycol at 60˜80° c. After cooling to 30˜40° C. there are added 35 l of polyethylene glycol and the mixture is stirred well. Then there is added a solution of 1750 grams of sodium saccharin in 2.5 l of purified water and while stirring there are added 2.5 l of cocoa flavor and polyethylene glycol q.s. to a volume of 50 l, providing an oral drop solution comprising 10 mg/ml of a.i. The resulting solution is filled into suitable containers.

### Example D.2

### Oral Solution

9 Grams of methyl 4-hydroxybenzoate and 1 gram of propyl 4-hydroxybenzoate are dissolved in 4 l of boiling purified water. In 3 l of this solution are dissolved first 10 grams of 2,3-dihydroxybutanedioic acid and thereafter 20 grams of the a.i. The latter solution is combined with the remaining part of the former solution and 12 1 1,2,3-propanetriol and 3 l of sorbitol 70% solution are added thereto. 40 Grams of sodium saccharin are dissolved in 0.5 l of water and 2 ml of raspberry and 2 ml of gooseberry essence are added. The latter solution is combined with the former, water is added q.s. to a volume of 20 l providing an oral solution comprising 5 mg of the active ingredient per teaspoonful (5 ml). The resulting solution is filled in suitable containers.

### Example D.3

### Film-Coated Tablets

### Preparation of Tablet Core

A mixture of 100 grams of the a.i., 570 grams lactose and 200 grams starch is mixed well and thereafter humidified with a solution of 5 grams sodium dodecyl sulfate and 10 grams polyvinylpyrrolidone in about 200 ml of water. The wet powder mixture is sieved, dried and sieved again. Then there is added 100 grams microcrystalline cellulose and 15 grams hydrogenated vegetable oil. The whole is mixed well and compressed into tablets, giving 10,000 tablets, each containing 10 mg of the active ingredient.

### Coating

To a solution of 10 grams methyl cellulose in 75 ml of denaturated ethanol there is added a solution of 5 grams of ethyl cellulose in 150 ml of dichloromethane. Then there are added 75 ml of dichloromethane and 2.5 ml 1,2,3-propanetriol. 10 grams of polyethylene glycol is molten and dissolved in 75 ml of dichloromethane. The latter solution is added to the former and then there are added 2.5 grams of magnesium octadecanoate, 5 grams of polyvinylpyrrolidone and 30 ml of concentrated color suspension and the whole is homogenated. The tablet cores are coated with the thus obtained mixture in a coating apparatus.

### Example D.4

### Injectable Solution

1.8 grams methyl 4-hydroxybenzoate and 0.2 grams propyl 4-hydroxybenzoate are dissolved in about 0.5 l of boiling water for injection. After cooling to about 50° C. there are added while stirring 4 grams lactic acid, 0.05 grams propylene glycol and 4 grams of the a.i. The solution is cooled to room temperature and supplemented with water for injection q.s. ad 1 l, giving a solution comprising 4 mg/ml of a.i. The solution is sterilized by filtration and filled in sterile containers.

### E. Physico-Chemical Data

### E.1 LCMS

The HPLC gradient was supplied by a HP 1100 from Agilent with a column heater set at 40° C. Flow from the column was passed through photodiode array (PDA) detector and then split to a MS detector that could be a ZQ or ToF (Time of Flight) mass spectrometer from Waters-Micromass. In the former case also a Light Scattering detector (ELSD) was installed. MS detectors were configured with an electrospray ionization source and could operate simultaneously in positive (at one or two voltages) and negative ionization mode or only in positive mode depending on the MS method applied.

LC Method in Reversed phase HPLC was carried out on a XDB-C18 cartridge (3.5 μm, 4.6×30 mm) from Agilent, with a flow rate of 1 ml/min. (named “S2011” for HPLC coupled with ToF and “S3011” for HPLC coupled with ZQ). Three mobile phases (mobile phase A: 0.5 g/l ammoniumacetate solution, mobile phase B: acetonitrile; mobile phase C: methanol) were employed to run a gradient condition from 80% A, 10% B, 10% C to 50% B and 50% C in 6.0 min., to 100% B at 6.5 min., kept till 7.0 min and reequilibrated with 80% A, 10% B and 10% C at 7.6 min. that was kept till 9.0 min. A 5 μL volume of the sample was injected.

Standard MS Method in ToF: High Resolution Mass spectra were acquired by scanning from 100 to 750 in 1 s. Nitrogen was used a the nebulizer gas. Cone voltage was 20 V for both positive and negative ionization mode. The source temperature was maintained at 140° C. Leucine-enkephaline was the reference used for the lock spray.

Standard MS Method in ZQ: Mass spectra were acquired by scanning from 100 to 1000 in 1 s. Nitrogen was used a the nebulizer gas. Cone voltage was 20 V for both positive and negative ionization mode. The source temperature was maintained at 140° C.

In both cases, Data acquisition was performed with a Waters-Micromass MassLynx-Openlynx data system.

TABLE 9
Physico-chemical data

| | Melting | LCMS | | | |
|---|---|---|---|---|---|
| Co. | Point | | | | Fragment/ |
| No. | (° C.) | Rₜ(Purity %) | MW | (MH)⁺ | Adduct |
| 1 | 249.8 | | | | |
| 8 | | 4.9 (100.00) | 514 | 515 | |
| 9 | | 5.36 (87.56) | 528 | 529 | |
| 10 | | 5.41 (97.44) | 542 | 543 | |
| 11 | | 3.36 (99.05) | 470 | 471 | |
| 15 | | 5.61 (92.66) | 499 | 500 | |
| 16 | >300 | 3.05 (99.60) | 359 | 360 | |
| 17 | 263.3 | | | | |
| 18 | 222.8 | 2.83 (97.92) | 437.24 | 438 | |
| 23 | | 3.45 (95.55) | 423 | 424 | |
| 24 | 260.6 | 3.55 (97.24) | 423.23 | 424 | 446 (MNa)⁺ |
| 25 | 268.4 | 3.68 (97.30) | 441 | 442 | |
| 28 | | 5.19 (97.19) | 461 | 462 | |
| 29 | 246.8 | 5.25 (100) | 461.28 | 462 | 484 (MNa)⁺ |
| 30 | | 5.71 (98.59) | 475.29 | 476 | |
| 31 | | 4.54 (89.43) | 460.30 | 461 | 331 (MH⁺ − 130) |
| 32 | | 3.74 (99.51) | 417.25 | 418 | |
| 33 | | 4.22 (98.82) | 403.24 | 404 | |
| 34 | | 4.34 (88.27) | 403.53 | 404 | 131 (MH⁺ − 273) |
| 35 | | 3.38 (88.60) | 464 | 465 | |
| 36 | | 2.75 (85.83) | 393 | 394 | |
| 37 | | 2.94 (94.81) | 423 | 424 | |
| 40 | 214.4 | 2.97 (100.00) | 478.31 | 479 | |
| 41 | 228.3 | 3.62 (95.82) | 488.33 | 489 | |
| 42 | | 5.78 (93.60) | 546 | 547 | |
| 43 | | 5.41 (97.75) | 540 | 541 | |
| 44 | | 2.83 (95.35) | 359 | 360 | |
| 45 | 259.7 | 3.92 (96.85) | 423 | 424 | |
| 51 | 220.8 | 3.74 (93.58) | 476.29 | 477 | 259 (MH⁺ − 218) |
| 52 | 122.9 | | | | |
| 55 | | 4.85 (91.66) | 568 | 569 | |
| 56 | | 5.16 (96.00) | 582 | 583 | |
| 57 | | 6.1 (93.54) | 564 | 565 | |
| 58 | | 5.4 (99.12) | 554 | 555 | |
| 59 | | 4.79 (87.34) | 540 | 541 | |
| 69 | | 5.2 (84.33) | 473 | 474 | |
| 75 | >300 | | | | |
| 79 | | 4.8 (88.71) | 409.28 | 410 | |
| 80 | | 5.6 (96.43) | 473.28 | 474 | 387 (MH⁺ − 87) |
| 81 | | 5.19 (88.69) | 487.29 | 488 | 358 (MH⁺ − 130) |
| 82 | | 4.06 (94.86) | 489.24 | 490 | 393 (MH⁺ − 97) |
| 83 | | 5.21 (94.47) | 485.24 | 486 | 508 (MNa)⁺ |
| 84 | | 4.06 (92.44) | 489.57 | 490 | |
| 85 | | 1.72 (98.90) | 367 | 368 | |
| 86 | | 2.47 (87.86) | 410 | 411 | |
| 97 | | 1.95 (99.48) | 379.2 | 380 | |
| 98 | | 5.87 (97.84) | 475 | 476 | |
| 99 | | 5.17 (96.54) | 487.58 | 488 | 510 (MNa)⁺ |
| 100 | | 3.16 (95.98) | 421.50 | 422 | |
| 101 | | 3.46 (97.45) | 457 | 458 | |
| 102 | | 2.85 (100.00) | 476 | 477 | |
| 103 | | 3.46 (99.23) | 421 | 422 | |
| 105 | | 5.22 (100.00) | 399 | 400 | |
| 106 | | 4.91 (79.49) | 442 | 443 | |
| 108 | | 3.7 (99.42) | 368.18 | 369 | |
| 123 | | 4.95 (91.82) | 560 | 561 | |
| 124 | | 4.11 (99.44) | 572 | 573 | |
| 125 | | 4.8 (98.56) | 554 | 555 | |
| 126 | | 3.42 (98.02) | 555 | 556 | |
| 127 | | 3.81 (96.82) | 548 | 549 | |
| 128 | | 4.12 (98.27) | 543 | 544 | |
| 130 | | 4.81 (99.5) | 560 | 561 | |
| 131 | | 5.79 (96.55) | 592 | 593 | |
| 132 | | 3.8 (96.6) | 400 | 401 | |

1. A compound according to the general Formula (I) 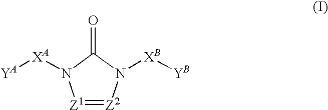
a pharmaceutically acceptable acid or base addition salt thereof, a stereochemically isomeric form thereof, an N-oxide form thereof or a quaternary ammonium salt thereof, wherein
Z¹ and Z² each independently from each other are CH or N;
X^{A} and X^{B} each independently from each other are a covalent bond or a C₁₋₄alkyl-radical, wherein one bivalent —CH₂-unit may be replaced by a bivalent phenyl-unit and/or wherein one or more hydrogen atoms in each moiety X^{A} and X^{B} may be replaced by a radical selected from the group of halo, cyano, hydroxy, amino, oxo and formyl;
Y^{A} and Y^{B} each independently from each other are a radical selected from the group of t-butyl, NR¹R² and Pir;
R¹ and R² each independently from each other are a radical selected from the group of hydrogen; alkyl; aryl; aryloxy; Het; —NR^{a}R^{b}, wherein R^{a} and R^{b} each independently are hydrogen, alkyl, aryl or arylalkyl; and
alkyl substituted with one or more radicals selected from the group of aryl, aryloxy, Het and —NR^{a}R^{b}, wherein R^{a} and R^{b} each independently are selected from the group of hydrogen, alkyl, aryl and arylalkyl;
Pir is a radical selected from the group of pyrrolyl; pyrazolyl; imidazolyl; pyridinyl; pyrimidinyl; pyrazinyl; pyridazinyl; pyrrolidinyl; imidazolidinyl; pyrrazolidinyl; piperidinyl; diazepyl; morpholinyl; thiomorpholinyl; piperazinyl; imidazolidinyl; 2H-pyrrolyl; pyrrolinyl; imidazolinyl; pyrrazolinyl; 1,2,3,4-tetrahydro-isoquinolinyl; 7,9-diaza-bicyclo[4.2.2]dec-3-enyl and isoindolyl; wherein each Pir-radical may optionally be substituted by one or more radicals selected from the group of hydroxy; oxo; alkyl; alkylcarbonyl; alkylsulphonyl; alkyloxycarbonyl; aryloxyalkyl; mono-arylaminoalkyl; aryl; arylalkyl; arylalkenyl; pyrrolidinyl; furylalkyl optionally substituted with 1 or 2 alkyl radicals; benzofurylalkyl; 2,3-dihydro-benzo[1,4]dioxylalkyl; quinolinylalkyl; benzothienylalkyl and indolylalkyl, optionally substituted with halo;
Het is a monocyclic heterocyclic radical selected from the group of pyrrolyl, pyrazolyl, imidazolyl, pyridinyl, pyrimidinyl, pyrazinyl, pyridazinyl, pyrrolidinyl, imidazolidinyl, pyrrazolidinyl, piperidinyl, diazepyl, morpholinyl, thiomorpholinyl, piperazinyl, imidazolidinyl, 2H-pyrrolyl, pyrrolinyl, imidazolinyl, pyrrazolinyl, furanyl, thienyl, oxazolyl, isoxazolyl, thiazolyl, thiadiazolyl, isothiazolyl, dioxolyl, dithianyl, tetrahydrofuryl, triazolyl and triazinyl; or a bicyclic heterocyclic radical selected from the group of quinolinyl, isoquinolinyl, 1,2,3,4-tetrahydro-isoquinolinyl, quinoxalinyl, indolyl, isoindolyl, benzimidazolyl, benzoxazolyl, benzisoxazolyl, benzothiazolyl, benzisothiazolyl, benzofuranyl, benzothienyl, benzopiperidinyl, chromenyl and imidazo[1,2-a]pyridinyl; wherein each Het-radical is optionally substituted with alkyl;
or two adjacent moieties X and Y may be fused together to form the bivalent radical 1,2,3,4-tetrahydro-isoquinolinyl, optionally substituted with hydrogen, alkyl, aryl, arylalkyl, alkylcarbonyl, alkylsulphonyl and pyrrolidinylalkyl;
aryl is naphthalenyl or phenyl, each optionally substituted with 1, 2 or 3 substituents, each independently from each other, selected from the group of halo, cyano, hydroxy, amino, alkylamino, alkyloxyalkylamino, oxo, carboxy, nitro, thio, formyl and alkyloxy;
alkyl is a straight or branched saturated hydrocarbon radical having from 1 to 6 carbon atoms; or is a cyclic saturated hydrocarbon (cycloalkyl) radical having from 3 to 7 carbon atoms; or is a cyclic saturated hydrocarbon radical having from 3 to 7 carbon atoms attached to a straight or branched saturated hydrocarbon radical having from 1 to 6 carbon atoms; each radical may optionally be substituted on one or more carbon atoms with one or more radicals selected from the group of halo, cyano, hydroxy, amino, oxo, carboxy, nitro, thio and formyl; and
alkenyl is an alkyl radical as defined above further having one or more double bonds.

2. A compound according to claim 1, wherein Z¹ is CH and Z² is N; or Z¹ is N and Z² is N; or Z¹ is CH and Z² is CH; or Z¹ is CH and Z² is CH.

3. A compound according to claim 2, wherein Z¹ is CH and Z² is N.

4. A compound of claim 1 wherein each of X^{A} and X^{B}, independently from each other is selected from the group of a covalent bond, —CH₂—, —CH₂CH₂—, —CH₂CH₂CH₂—, —CH₂CH₂CH₂CH₂—, —CH(CH₃)CH(CH₃)—, —C(═O)CH₂—, —CH₂C(═O)—, —CH₂CH₂C(═O)CH₂—, —C₆H₄—, —CH₂C₆H₅—, —CH₂CH₂C₆H₅—, —C₆H₅CH₂—, —C₆H₅CH₂CH₂—, —CH₂C₆H₄CH₂— and —CH₂CH₂C₆H₄CH₂CH₂—.

5. A compound according to claim 4, wherein each of X^{A} and X^{B}, independently from each other is selected from the group of acovalent bond, —CH₂—, —CH₂CH₂—, —CH₂CH₂CH₂—, —CH(CH₃)CH(CH₃)—, —C(═O)CH₂—, —CH₂CH₂C(═O)CH₂—, —C₆H₄—, —CH₂C₆H₅—, —C₆H₅CH₂— and —CH₂C₆H₄CH₂—.

6. A compound according to claim 5, wherein X^{A} is —C₆H₅CH₂— and X is —CH₂CH₂—.

7. A compound according to claim 1 wherein Y^{A} is NR¹R² and Y^{B} is Pir; or Y^{A} is NR¹R² and Y^{B} is NR¹R²; or Y^{A} is Pir and Y^{B} is Pir; or Y^{A} is Pir and Y^{B} is NR¹R².

8. A compound according to claim 7, wherein Y^{A} is Pir and Y^{B} is NR¹R².

9. A compound according to claim 1 wherein Pir is selected from the group of pyrrolidinyl; piperidinyl; diazepyl; morpholinyl; piperazinyl; 1,2,3,4etrahydro-isoquinolinyl; 7,9-diaza-bicyclo[4.2.2]dec-3-enyl and isoindolyl; wherein each Pir-radical may optionally be substituted by one or more radicals selected from the group of hydroxy; oxo; alkyl; alkyloxycarbonyl; aryloxyalkyl; mono-arylaminoalkyl, aryl; arylalkyl; arylalkenyl; pyrrolidinyl; furylalkyl, optionally substituted with 1 or 2 alkyl radicals; benzofurylalkyl 2,3-dihydro-benzo[1,4]dioxylalkyl; quinolinylalkyl, benzothienylalkyl and indolylalkyl, optionally substituted with halo.

10. A compound according to claim 9, wherein Pir is morpholinyl.

11. A compound according to claim 1 wherein each of R¹ and R² independently from each other are selected from the group of hydrogen; alkyl; aryl and alkyl substituted with a radical selected from the group of aryl, aryloxy, Het and —NR^{a}R^{b}, wherein R^{a} and R^{b} each independently are selected from the group of hydrogen, alkyl and arylalkyl.

12. A compound according to claim 11, wherein R¹ and R², independently from each other are selected from the group of hydrogen; methyl; ethyl; phenyl; and methyl and ethyl, substituted with a radical selected from the group of phenyl, phenyloxy, dimethylamino, (benzyl)(methyl)amino, (cyclohexylmethyl)(methyl)amino, pyridinyl, pyrrolidinyl, piperidinyl, morpholinyl, piperazinyl and tetrahydrofuryl.

13. A compound according to claim 12 wherein R¹ and R², independently from each other are selected from the group of hydrogen and phenyloxyethyl.

14. A compound according to claim 1 wherein
Z¹ and Z² each independently from each other, are CH or N;
X^{A} and X^{B} each independently from each other, are a covalent bond or a C₁₋₄alkyl-radical, wherein one bivalent —CH₂-unit may be replaced by a bivalent phenyl-unit and wherein one or more hydrogen atoms in each moiety X^{A} and X^{B} may be replaced by an oxo radical;
Y^{A} and Y^{B} each independently from each other are a radical selected from the group of t-butyl, NR¹R² and Pir;
R¹ and R² independently from each other are selected from the group of hydrogen; alkyl; aryl and alkyl substituted with a radical selected from the group of aryl, aryloxy, Het and —NR^{a}R^{b}, wherein R^{a} and R^{b} each independently are selected from the group of hydrogen, alkyl and arylalkyl;
Pir is a heterocyclic radical selected from the group of pyrrolidinyl piperidinyl; diazepyl; morpholinyl; piperazinyl; 1,2,3,4-tetrahydro-isoquinolinyl; 7,9-diaza-bicyclo[4.2.2]dec-3-enyl and isoindolyl; wherein each Pir-radical may optionally be substituted by one or more radicals selected from the group of hydroxy; oxo; alkyl; alkyloxycarbonyl; aryloxyalkyl; mono-arylaminoalkyl, aryl; arylalkyl; arylalkenyl; pyrrolidinyl; furylalkyl, optionally substituted with 1 or 2 alkyl radicals; benzofurylalkyl; 2,3-dihydro-benzo[1,4]dioxylalkyl; quinolinylalkyl, benzothienylalkyl and indolylalkyl, optionally substituted with halo.
Het is a monocyclic heterocyclic radical selected from the group of pyridinyl, pyrrolidinyl, piperidinyl, morpholinyl, piperazinyl and tetrahydrofuryl;
or two adjacent X and Y moieties may form together the bivalent radical 1,2,3,4-tetrahydro-isoquinolinyl, optionally substituted with hydrogen, alkyl, arylalkyl, alkylcarbonyl, alkylsulphonyl and pyrrolidinylalkyl; and
aryl is naphthalenyl or phenyl, each optionally substituted with 1, 2 or 3 substituents, each independently from each other, selected from the group of halo, cyano, hydroxy and alkyloxy.

15. A compound according to claim 1 wherein aryloxyalkyl is phenyloxyethyl, arylalkenyl is 2-methyl-3-phenyl-allyl and isoindolyl is substituted with two oxo-moieties to form an isoindole-1,3-dionyl-moiety.

16. (canceled)

17. A compound according to claim 1, wherein the compound is 4-(4-morpholin-4-ylmethyl-phenyl)-2-[2-(2-phenoxy-ethylamino)-ethyl]-2,4-dihydro-[1,2,4]triazol-3-on, a pharmaceutically acceptable acid or base addition salt thereof, a stereochemically isomeric form thereof, an N-oxide form thereof or a quaternary ammonium salt thereof.

18. (canceled)

19. (canceled)

20. A pharmaceutical composition comprising a pharmaceutically acceptable carrier or diluent and, as active ingredient, a therapeutically effective amount of a compound according to claim 1.

21. A pharmaceutical composition according to claim 20, wherein the pharmaceutical composition further comprises one or more other compounds selected from the group of antidepressants, anxiolytics and antipsychotics.

22. A pharmaceutical composition according to claim 20 wherein the pharmaceutical composition is in a form suitable to be orally administered.

23. A process for the preparation of a pharmaceutical composition as claimed in claim 20, wherein a pharmaceutically acceptable carrier is intimately mixed with a therapeutically effective amount of a compound as claimed in claim 1.

24. A process for the preparation of a pharmaceutical composition as claimed in claim 21, wherein pharmaceutically acceptable carrier is intimately mixed with a therapeutically effective amount of a compound as claimed in claim 17 and one or more other compounds selected from the group of antidepressants, anxiolytics and antipsychotics.

25. A method for the prevention and/or treatment of diseases where antagonism of the α₂-adrenergic receptor, in particular antagonism of the α_{2C}-adrenergic receptor is of therapeutic use comprising administering a therapeutically effective amount of the compound of claims 1 to a patient in need of treatment.

26. A method for the prevention and/or treatment of central nervous system disorders, mood disorders, anxiety disorders, stress-related disorders associated with depression and/or anxiety, cognitive disorders, personality disorders, schizoaffective disorders, Parkinson's disease, dementia of the Alzheimer's type, chronic pain conditions, neurodegenerative diseases, addiction disorders, mood disorders and sexual dysfunction in a patient comprising administering a therapeutically effective amount of the compound of claim 1 to a patient.

27. The method of claim 25 wherein additional administered is one or more other compounds selected from the group of antidepressants, anxiolytics and antipsychotics.

28. Process for the preparation of a compound according to claim 1, wherein a compound of Formula (I′) is converted into a compound according to Formula (I), 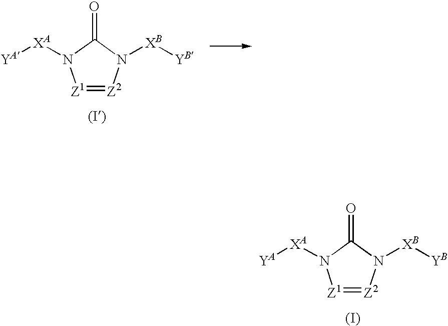
wherein all variables are defined as in claim 1 and wherein either at least one of Y^{A′} and Y^{B′} is selected from the group of halo; formyl; alkylSO₃; cyano; hydroxy; and alkyloxy, and ethyloxy; or wherein at least one of Y^{A′} and Y^{B′} is NR¹L^{B}, NL^{A}R² or NL^{A}L^{B}, wherein L^{A} and L^{B} are each independently of each other selected from the group of alkyloxycarbonyl,; and arylalkyloxycarbonyl.

29. Intermediate compound according to Formula (I′) 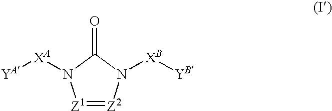
wherein at least one of Y^{A′} and Y^{B′} is selected from the group of halo; formyl; alkylSO₃—; cyano; hydroxy; and alkyloxy.

30. Intermediate compound according to Formula (I′) wherein at least one of Y^{A′} and Y^{B′} is NR¹L^{B}, NL^{A}R² or NL^{A}L^{B}, wherein L^{A} and L^{B} are each independently of each other selected from the group of alkyloxycarbonyl; and arylalkyloxycarbonyl.
